(19) United States
(12) Patent Application Publication
(10) Pub. No.: US 2025/0152074 A1
(43) Pub. Date: May 15, 2025

(54) Mobile Wearable Electromagnetic Brain Activity Monitor

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 19/020,737

(22) Filed: Jan. 14, 2025

Related U.S. Application Data
(63) Continuation-in-part of application No. 18/944,224, filed on Nov. 12, 2024.
(63) Continuation-in-part of application No. 18/902,821, filed on Sep. 30, 2024.
(63) Continuation-in-part of application No. 18/748,059, filed on Jun. 19, 2024, now Pat. No. 12,201,427.
(63) Continuation-in-part of application No. 18/411,540, filed on Jan. 12, 2024.
Application No. 18/944,224 is a continuation-in-part of application No. 18/902,821, filed on Sep. 30, 2024.
Application No. 18/902,821 is a continuation-in-part of application No. 18/748,059, filed on Jun. 19, 2024, now Pat. No. 12,201,427.
Application No. 18/748,059 is a continuation-in-part of application No. 18/411,540, filed on Jan. 12, 2024.
Application No. 18/411,540 is a continuation-in-part of application No. 18/219,684, filed on Jul. 9, 2023, now abandoned.
Application No. 18/902,821 is a continuation-in-part of application No. 18/748,059, filed on Jun. 19, 2024, now Pat. No. 12,201,427.
Application No. 18/748,059 is a continuation-in-part of application No. 18/411,540, filed on Jan. 12, 2024.
Application No. 18/411,540 is a continuation-in-part of application No. 18/219,684, filed on Jul. 9, 2023, now abandoned.
Application No. 18/748,059 is a continuation-in-part of application No. 18/411,540, filed on Jan. 12, 2024.
Application No. 18/411,540 is a continuation-in-part of application No. 18/219,684, filed on Jul. 9, 2023, now abandoned.
Application No. 18/411,540 is a continuation-in-part of application No. 18/219,684, filed on Jul. 9, 2023, now abandoned.
Application No. 18/219,684 is a continuation-in-part of application No. 17/714,988, filed on Apr. 6, 2022, now Pat. No. 11,850,052.
Application No. 17/714,988 is a continuation-in-part of application No. 16/838,541, filed on Apr. 2, 2020, now abandoned.
Application No. 17/714,988 is a continuation-in-part of application No. 17/665,086, filed on Feb. 4, 2022, now Pat. No. 11,662,819.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/136,117 is a continuation-in-part of application No. 16/838,541, filed on Apr. 2, 2020, now abandoned.
Application No. 16/838,541 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/554,029, filed on Aug. 28, 2019, now abandoned.
Application No. 16/554,029 is a continuation-in-part of application No. 16/554,029, filed on Aug. 28, 2019, now abandoned.
Application No. 16/554,029 is a continuation-in-part of application No. 15/236,401, filed on Aug. 13, 2016, now abandoned.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 16/568,580 is a continuation-in-part of application No. 15/963,061, filed on Apr. 25, 2018, now Pat. No. 10,772,559.
Application No. 15/963,061 is a continuation-in-part of application No. 16/022,987, filed on Jun. 29, 2018, now Pat. No. 11,172,859.
Application No. 16/022,987 is a continuation-in-part of application No. 15/136,948, filed on Apr. 24, 2016, now Pat. No. 10,234,942.
Application No. 15/963,061 is a continuation-in-part of application No. 15/464,349, filed on Mar. 21, 2017, now Pat. No. 9,968,297.
Application No. 15/464,349 is a continuation-in-part of application No. 15/136,948, filed on Apr. 24, 2016, now Pat. No. 10,234,942.
Application No. 15/136,948 is a continuation-in-part of application No. 14/562,719, filed on Dec. 7, 2014, now Pat. No. 10,130,277.
Application No. 14/562,719 is a continuation-in-part of application No. 14/330,649, filed on Jul. 14, 2014, now abandoned.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/330,649 is a continuation-in-part of application No. 13/797,955, filed on Mar. 12, 2013, now Pat. No. 9,456,916.
Application No. 13/797,955 is a continuation-in-part of application No. 13/523,739, filed on Jun. 14, 2012, now Pat. No. 9,042,596.
(60) Provisional application No. 62/972,692, filed on Feb. 11, 2020.
(60) Provisional application No. 62/972,692, filed on Feb. 11, 2020.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/430,667, filed on Dec. 6, 2016.
(60) Provisional application No. 62/322,594, filed on Apr. 14, 2016.
(60) Provisional application No. 62/303,126, filed on Mar. 3, 2016.
(60) Provisional application No. 62/169,661, filed on Jun. 2, 2015.
(60) Provisional application No. 62/160,172, filed on May 12, 2015.
(60) Provisional application No. 62/089,696, filed on Dec. 9, 2014.
(60) Provisional application No. 62/017,615, filed on Jun. 26, 2014.
(60) Provisional application No. 61/939,244, filed on Feb. 12, 2014.
(60) Provisional application No. 61/932,517, filed on Jan. 28, 2014.
(60) Provisional application No. 61/932,517, filed on Jan. 28, 2014.
(60) Provisional application No. 61/729,494, filed on Nov. 23, 2012.

(51) Int. Cl. A61B 5/369; A61B 5/00

(52) U.S. Cl. CPC A61B 5/369; A61B 5/6817

(57) ABSTRACT

A wearable brain activity monitor with electromagnetic sensors can be embodied in an ear-worn device. A first portion of the device can span a portion of the lateral perimeter of a person's ear and a second portion of the device can be inserted into the person's ear canal. In an example, the first portion can loop around 25% to 75% of the lateral perimeter of the person's ear.

### CROSS-REFERENCE TO RELATED APPLICATIONS

This patent application is a continuation-in-part of U.S. patent application Ser. No. 18/944,224 filed on 2024 Nov. 12. This patent application is a continuation-in-part of U.S. patent application Ser. No. 18/902,821 filed on 2024 Sep. 30. This patent application is a continuation-in-part of U.S. patent application Ser. No. 18/748,059 filed on 2024 Jun. 19. This patent application is a continuation-in-part of U.S. patent application Ser. No. 18/411,540 filed on 2024 Jan. 12.

U.S. patent application Ser. No. 18/944,224 was a continuation-in-part of U.S. patent application Ser. No. 18/902,821 filed on 2024 Sep. 30. U.S. patent application Ser. No. 18/944,224 was a continuation-in-part of U.S. patent application Ser. No. 18/748,059 filed on 2024 Jun. 19. U.S. patent application Ser. No. 18/944,224 was a continuation-in-part of U.S. patent application Ser. No. 18/411,540 filed on 2024 Jan. 12. U.S. patent application Ser. No. 18/944,224 was a continuation-in-part of U.S. patent application Ser. No. 18/219,684 filed on 2023 Jul. 9.

U.S. patent application Ser. No. 18/902,821 was a continuation-in-part of U.S. patent application Ser. No. 18/748,059 filed on 2024 Jun. 19. U.S. patent application Ser. No. 18/902,821 was a continuation-in-part of U.S. patent application Ser. No. 18/411,540 filed on 2024 Jan. 12. U.S. patent application Ser. No. 18/902,821 was a continuation-in-part of U.S. patent application Ser. No. 18/219,684 filed on 2023 Jul. 9.

U.S. patent application Ser. No. 18/748,059 was a continuation-in-part of U.S. patent application Ser. No. 18/411,540 filed on 2024 Jan. 12. U.S. patent application Ser. No. 18/748,059 was a continuation-in-part of U.S. patent application Ser. No. 18/219,684 filed on 2023 Jul. 9. U.S. patent application Ser. No. 18/411,540 was a continuation-in-part of U.S. patent application Ser. No. 18/219,684 filed on 2023 Jul. 9. U.S. patent application Ser. No. 18/219,684 was a continuation-in-part of U.S. patent application Ser. No. 17/714,988 filed on 2022 Apr. 6. U.S. patent application Ser. No. 18/219,684 was a continuation-in-part of U.S. patent application Ser. No. 16/838,541 filed on 2020 Apr. 2.

U.S. patent application Ser. No. 17/714,988 was a continuation-in-part of U.S. patent application Ser. No. 17/665,086 filed on 2022 Feb. 4. U.S. patent application Ser. No. 17/714,988 was a continuation-in-part of U.S. patent application Ser. No. 17/136,117 filed on 2020 Dec. 29. U.S. patent application Ser. No. 17/714,988 was a continuation-in-part of U.S. patent application Ser. No. 16/554,029 filed on 2019 Aug. 28. U.S. patent application Ser. No. 17/665,086 was a continuation-in-part of U.S. patent application Ser. No. 17/136,117 filed on 2020 Dec. 29. U.S. patent application Ser. No. 17/665,086 was a continuation-in-part of U.S. patent application Ser. No. 16/554,029 filed on 2019 Aug. 28.

U.S. patent application Ser. No. 17/136,117 was a continuation-in-part of U.S. patent application Ser. No. 16/838,541 filed on 2020 Apr. 2. U.S. patent application Ser. No. 17/136,117 claimed the priority benefit of U.S. provisional patent application 62/972,692 filed on 2020 Feb. 11. U.S. patent application Ser. No. 17/136,117 was a continuation-in-part of U.S. patent application Ser. No. 16/737,052 filed on 2020 Jan. 8. U.S. patent application Ser. No. 17/136,117 was a continuation-in-part of U.S. patent application Ser. No. 16/568,580 filed on 2019 Sep. 12. U.S. patent application Ser. No. 17/136,117 was a continuation-in-part of U.S. patent application Ser. No. 16/554,029 filed on 2019 Aug. 28.

U.S. patent application Ser. No. 16/838,541 claimed the priority benefit of U.S. provisional patent application 62/972,692 filed on 2020 Feb. 11. U.S. patent application Ser. No. 16/838,541 was a continuation-in-part of U.S. patent application Ser. No. 16/554,029 filed on 2019 Aug. 28. U.S. patent application Ser. No. 16/838,541 claimed the priority benefit of U.S. provisional patent application 62/851,917 filed on 2019 May 23. U.S. patent application Ser. No. 16/838,541 claimed the priority benefit of U.S. provisional patent application 62/837,712 filed on 2019 Apr. 23. U.S. patent application Ser. No. 16/838,541 was a continuation-in-part of U.S. patent application Ser. No. 15/236,401 filed on 2016 Aug. 13. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25. U.S. patent application Ser. No. 16/568,580 was a continuation-in-part of U.S. patent application Ser. No. 15/963,061 filed on 2018 Apr. 25.

U.S. patent application Ser. No. 16/554,029 claimed the priority benefit of U.S. provisional patent application 62/851,904 filed on 2019 May 23. U.S. patent application Ser. No. 16/554,029 claimed the priority benefit of U.S. provisional patent application 62/796,901 filed on 2019 Jan. 25. U.S. patent application Ser. No. 16/554,029 claimed the priority benefit of U.S. provisional patent application 62/791,838 filed on 2019 Jan. 13. U.S. patent application Ser. No. 16/554,029 was a continuation-in-part of U.S. patent application Ser. No. 16/022,987 filed on 2018 Jun. 29. U.S. patent application Ser. No. 16/022,987 was a continuation-in-part of U.S. patent application Ser. No. 15/136,948 filed on 2016 Apr. 24. U.S. patent application Ser. No. 15/963,061 was a continuation-in-part of U.S. patent application Ser. No. 15/464,349 filed on 2017 Mar. 21.

U.S. patent application Ser. No. 15/464,349 claimed the priority benefit of U.S. provisional patent application 62/430,667 filed on 2016 Dec. 6. U.S. patent application Ser. No. 15/464,349 was a continuation-in-part of U.S. patent application Ser. No. 15/136,948 filed on 2016 Apr. 24. U.S. patent application Ser. No. 15/464,349 was a continuation-in-part of U.S. patent application Ser. No. 14/562,719 filed on 2014 Dec. 7. U.S. patent application Ser. No. 15/464,349 was a continuation-in-part of U.S. patent application Ser. No. 14/330,649 filed on 2014 Jul. 14. U.S. patent application Ser. No. 15/236,401 was a continuation-in-part of U.S. patent application Ser. No. 15/136,948 filed on 2016 Apr. 24. U.S. patent application Ser. No. 15/236,401 was a continuation-in-part of U.S. patent application Ser. No. 14/599,522 filed on 2015 Jan. 18.

U.S. patent application Ser. No. 15/136,948 claimed the priority benefit of U.S. provisional patent application 62/322,594 filed on 2016 Apr. 14. U.S. patent application Ser. No. 15/136,948 claimed the priority benefit of U.S. provisional patent application 62/303,126 filed on 2016 Mar. 3. U.S. patent application Ser. No. 15/136,948 claimed the priority benefit of U.S. provisional patent application 62/169,661 filed on 2015 Jun. 2. U.S. patent application Ser. No. 15/136,948 claimed the priority benefit of U.S. provisional patent application 62/160,172 filed on 2015 May 12. U.S. patent application Ser. No. 15/136,948 was a continuation-in-part of U.S. patent application Ser. No. 14/599,522 filed on 2015 Jan. 18.

U.S. patent application Ser. No. 14/599,522 claimed the priority benefit of U.S. provisional patent application 62/089,696 filed on 2014 Dec. 9. U.S. patent application Ser. No. 14/599,522 was a continuation-in-part of U.S. patent application Ser. No. 14/562,719 filed on 2014 Dec. 7. U.S. patent application Ser. No. 14/599,522 claimed the priority benefit of U.S. provisional patent application 62/017,615 filed on 2014 Jun. 26. U.S. patent application Ser. No. 14/599,522 claimed the priority benefit of U.S. provisional patent application 61/939,244 filed on 2014 Feb. 12. U.S. patent application Ser. No. 14/599,522 claimed the priority benefit of U.S. provisional patent application 61/932,517 filed on 2014 Jan. 28.

U.S. patent application Ser. No. 14/562,719 claimed the priority benefit of U.S. provisional patent application 61/932,517 filed on 2014 Jan. 28. U.S. patent application Ser. No. 14/330,649 was a continuation-in-part of U.S. patent application Ser. No. 13/797,955 filed on 2013 Mar. 12. U.S. patent application Ser. No. 14/330,649 was a continuation-in-part of U.S. patent application Ser. No. 13/523,739 filed on 2012 Jun. 14. U.S. patent application Ser. No. 13/797,955 claimed the priority benefit of U.S. provisional patent application 61/729,494 filed on 2012 Nov. 23.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable EEG (electroencephalographic) monitoring devices.

### Introduction

There are numerous potential applications for incorporating electrodes into devices which are worn on a person's head. Such devices can record brainwave activity (e.g. electroencephalographic data) for use as a BCI (Brain-to-Computer Interface) for communication. Such devices can also be used for biometric monitoring to improve a person's health or to detect adverse heath events (e.g. seizures, strokes, or heart attacks).

### Review of the Relevant Art

U.S. patent application No. 20080146892 (Leboeuf et al., Jun. 19, 2008, “Physiological and Environmental Monitoring Systems and Methods”) discloses noninvasive health monitors including compact sensors integrated within small, low-profile devices. U.S. patent application No. 20080146890 (Leboeuf et al., Jun. 19, 2008, “Telemetric Apparatus for Health and Environmental Monitoring”) discloses noninvasive health and environmental monitors include a plurality of compact sensors integrated within small, low-profile devices, such as earpiece modules. U.S. patent application 20100217099 (Leboeuf et al., Aug. 26, 2010, “Methods and Apparatus for Assessing Physiological Conditions”) discloses a monitoring apparatus and methods for assessing a physiological condition of a subject.

U.S. patent application No. 20100217100 (Leboeuf et al., Aug. 26, 2010, “Methods and Apparatus for Measuring Physiological Conditions”) discloses a monitoring apparatus with a housing configured to be attached to an ear of a subject, and a plurality of electrodes supported by the housing. U.S. patent application No. 20110098112 (Leboeuf et al., Apr. 28, 2011, “Physiological and Environmental Monitoring Systems and Methods”) discloses noninvasive health monitors including compact sensors integrated within small, low-profile devices. U.S. patent application No. 20110106627 (Leboeuf et al., May 5, 2011, “Physiological and Environmental Monitoring Systems and Methods”) discloses noninvasive health monitors including compact sensors integrated within small, low-profile devices.

U.S. Pat. No. 8,157,730 (Leboeuf et al., Apr. 17, 2012, “Physiological and Environmental Monitoring Systems and Methods”) discloses noninvasive health monitors with a plurality of compact sensors integrated within small, low-profile devices. U.S. Pat. No. 8,204,786 (Leboeuf et al., Jun. 19, 2012, “Physiological and Environmental Monitoring Systems and Methods”) discloses systems and methods for monitoring various physiological and environmental factors. U.S. patent application No. 20120177233 (Kidmose et al., Jul. 12, 2012, “Hearing Aid Adapted for Detecting Brain Waves and a Method for Adapting Such a Hearing Aid”) discloses a hearing aid comprising an amplifier, an input transducer, an output transducer, and a signal processing device.

U.S. patent application No. 20120203081 (Leboeuf et al., Aug. 9, 2012, “Physiological and Environmental Monitoring Apparatus and Systems”) discloses systems and methods for monitoring various physiological and environmental factors. U.S. patent application No. 20120209101 (Kidmose et al., Aug. 16, 2012, “Ear Plug with Surface Electrodes”) discloses an ear plug comprising a shell with at least two electrodes adapted for measuring brain wave signals. U.S. patent application 20120238856 (Kidmose et al., Sep. 20, 2012, “Portable Monitoring Device with Hearing Aid and EEG Monitor”) discloses a method of monitoring an EEG signal of a hearing impaired person.

U.S. patent application No. 20120302858 (Kidmose et al., Nov. 29, 2012, “Portable EEG Monitor System with Wireless Communication”) discloses a system for remote surveillance of an EEG signal of a person susceptible of having a hypoglycemic seizure comprising an EEG sensor part with electrodes for measuring one or more EEG signals from the person, and a processing unit adapted to be arranged at the ear of said person. U.S. patent application No. 20130296731 (Kidmose et al., Nov. 7, 2013, “Personal EEG Monitoring Device with Electrode Validation”) discloses a personal wearable EEG monitor which is carried at the head of a person.

### SUMMARY OF THE INVENTION

A wearable brain activity monitor with electromagnetic sensors can be embodied in an ear-worn device. A first portion of the device can span a portion of the lateral perimeter of a person's ear and a second portion of the device can be inserted into the person's ear canal. In an example, the first portion can loop around 25% to 75% of the lateral perimeter of the person's ear.

### BRIEF INTRODUCTION TO THE FIGURES

FIG. 1 shows a wearable brain activity monitor which spans a portion of the lateral perimeter of a person's ear.

FIG. 2 shows a wearable brain activity monitor which spans a portion of the lateral perimeter of a person's ear and is also inserted into the person's ear canal.

### DETAILED DESCRIPTION OF THE FIGURES

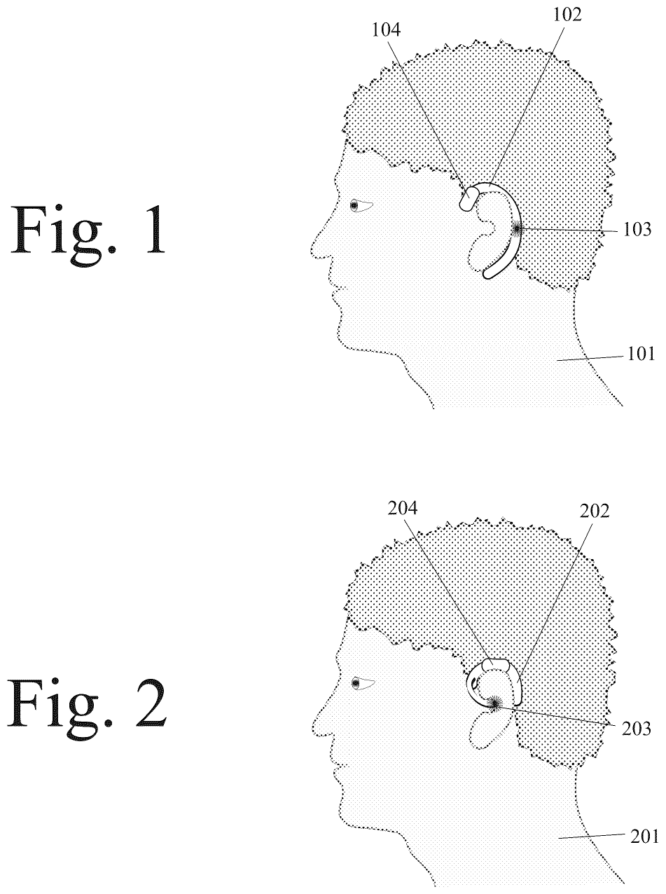

FIG. 1 shows a left-side view of an example of a wearable brain activity monitor comprising a head-worn sensor-positioning member 102 which is configured to position a plurality of electrodes or other brain activity sensors, including 103, at selected locations on the wearer's head 101. This monitor further comprises control unit 104. In an example, the monitor can be symmetric, with a similarly-configured sensor-positioning member on the right side of the wearer's head. In an alternative example, the monitor can asymmetric, with no sensor-positioning member on the wearer's right side.

In this example, sensor-positioning member 102 snuggly loops around a portion of the lateral perimeter of the wearer's ear. In this example, sensor-positioning member 102 loops around approximately 70% of the lateral perimeter of the wearer's ear. In various examples, a sensor-positioning member can loop around a percentage of the lateral perimeter of the wearer's ear in the range of 50% to 80%. In an example, the polar coordinates of the lateral perimeter of the wearer's ear can be expressed in terms of positions on a clockface. In this example, sensor positioning member 102 loops from approximately the 10 o'clock position to the 6 o'clock position. In various examples, a sensor-positioning member can loop around the ear within the range of 9 o'clock to 6 o'clock.

In this example, the loop spans a lower portion of the wearer's temporal lobe and a portion of their cerebellum. In an example, electrodes or other brain activity sensors collect data on brain activity concerning short term memory, smell, taste, vision and hearing. This brain activity data can be associated with selected quantities and types of food consumption and/or can be used to identify quantities and types of food consumed.

In an example, a wearable brain activity monitor can comprise an array of four electrodes or other brain activity sensors which are located substantially at the following set of placement sites—T7, T8, TP7 and TP8—or which comprise a subset of two or more sites from this set of placement sites.

In an example, control unit 104 can further comprise: a data processing component and a power source (or transducer). In an example, control unit 104 can further comprise: a data processing component; a power source (or transducer); and a data transmitting (and receiving) component. In an example, control unit 104 can be in wireless communication with an external (or remote) device and/or with another component of an overall system for monitoring brain activity. In an example, control unit 104 can further comprise: a data processing component; a power source (or transducer); a data transmitting (and receiving) component; and a user interface. In an example, control unit 104 can be physically connected to the array of electrodes (or other brain activity sensors) by wires or other electromagnetically-conductive pathways. In an example, control unit 104 can be in wireless electromagnetic communication with the array of electrodes (or other brain activity sensors).

In an example, this invention can be embodied in a device for measuring and/or modifying a person's food consumption comprising: a hearing aid or ear bud which is configured to be worn in, worn on, and/or worn around a person's ear; one or more electrodes or other brain activity sensors which are configured by the hearing aid or ear bud to be less than one inch from the surface of the person's head; a mobile power source and/or power transducer, wherein a power transducer harvests power from human physiological activity and/or environmental energy sources; a data processor; and a data transmitting member.

In an example, this invention can be embodied in a device for measuring and/or modifying a person's food consumption comprising: a hearing aid or ear bud which is configured to be worn in, worn on, and/or worn around a person's ear; one or more electrodes or other brain activity sensors which are configured by the hearing aid or ear bud to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a wearable brain activity monitor can comprise a head-worn sensor-positioning member which is configured to position a plurality of electrodes or other brain activity sensors at selected locations on a person's head, wherein the sensor-positioning member snuggly loops around a portion of a lateral perimeter of the person's ear. In an example, the sensor-positioning member can loop around a percentage of the lateral perimeter of the person's ear in the range of 50% to 80%. In an example, the sensor-positioning member can loop around the person's ear from the 10 o'clock position to the 6 o'clock position. In an example, the sensor-positioning member can loop around the person's ear within the range of the 9 o'clock position to the 6 o'clock position. In an example, the monitor can comprise two electrodes or other brain activity sensors at locations selected from the following set of placement sites: T7, T8, TP7 and TP8. Relevant example variations discussed elsewhere in this disclosure or in priority-linked disclosures can also be applied to this example.

FIG. 2 shows a left-side view of an example of a wearable brain activity monitor comprising a head-worn sensor-positioning member 202 which is configured to position a plurality of electrodes or other brain activity sensors, including 203, at selected locations on the wearer's head 201. This monitor further comprises control unit 204. In an example, the selected location from which an electromagnetic energy sensor collects data concerning electromagnetic brain activity can be on a person's ear and/or within the person's ear canal. In an example, the monitor can be symmetric, with a similarly-configured sensor-positioning member on the right side of the wearer's head. In an alternative example, the monitor can asymmetric, with no sensor-positioning member on the wearer's right side.

In this example, sensor-positioning member 202 snuggly loops around a portion of the lateral perimeter of the wearer's ear and also fits into the wearer's ear canal. In an example, at least one electrode or other brain activity sensor is configured to be within the wearer's ear canal. In this example, sensor-positioning member 202 loops around approximately 50% of the lateral perimeter of the wearer's ear. In various examples, a sensor-positioning member can loop around a percentage of the lateral perimeter of the wearer's ear in the range of 25% to 70%. In an example, the polar coordinates of the lateral perimeter of the wearer's ear can be expressed in terms of positions on a clockface. In this example, sensor positioning member 202 loops from approximately the 9 o'clock position to the 3 o'clock position. In various examples, a sensor-positioning member can loop around the ear within the range of 9 o'clock to 5 o'clock.

In this example, the loop spans a lower portion of the wearer's temporal lobe and a portion of their cerebellum. In an example, electrodes or other brain activity sensors collect data on brain activity concerning short term memory, smell, taste, vision and hearing. This brain activity data can be associated with selected quantities and types of food consumption and/or can be used to identify quantities and types of food consumed. In an example, a wearable brain activity monitor can comprise an array of two electrodes or other brain activity sensors which are located substantially at the following set of placement sites—TP7 and TP8.

In an example, control unit 204 can further comprise: a data processing component and a power source (or transducer). In an example, control unit 204 can further comprise: a data processing component; a power source (or transducer); and a data transmitting (and receiving) component. In an example, control unit 204 can be in wireless communication with an external (or remote) device and/or with another component of an overall system for monitoring brain activity. In an example, control unit 204 can further comprise: a data processing component; a power source (or transducer); a data transmitting (and receiving) component; and a user interface. In an example, control unit 204 can be physically connected to the array of electrodes (or other brain activity sensors) by wires or other electromagnetically-conductive pathways. In an example, control unit 204 can be in wireless electromagnetic communication with the array of electrodes (or other brain activity sensors).

In an example, a wearable brain activity monitor can comprise a head-worn sensor-positioning member which is configured to position a plurality of electrodes or other brain activity sensors at selected locations on a person's head, wherein the selected locations are on the person's ear and/or within the person's ear canal. In an example, the sensor-positioning member can snuggly loop around a portion of the lateral perimeter of the person's ear and also fits into the person's ear canal. In an example, the sensor-positioning member can loop around a percentage of the lateral perimeter of the person's ear in the range of 25% to 70%. In an example, the sensor-positioning member can loop around the person's ear from the 9 o'clock position to the 3 o'clock position. In an example, the sensor-positioning member can loop around the person's ear within the range of the 9 o'clock position to the 5 o'clock position. In an example, the monitor can comprise two electrodes or other brain activity sensors at locations selected from the following set of placement sites: TP7 and TP8. In an example, at least one electrode or other brain activity sensor can be configured to be within the person's ear canal. In an example, the monitor can be embodied in an ear bud. In an example, the monitor can be embodied in a hearing aid. Relevant example variations discussed elsewhere in this disclosure or in priority-linked disclosures can also be applied to this example.

In an example, a mobile wearable electromagnetic brain activity monitor can comprise: a wearable frame worn on a person's head; a plurality of electromagnetic energy sensors which collect data concerning the person's electromagnetic brain activity; and a control unit. In an example, electromagnetic energy sensors can be electroencephalogram (EEG) electrodes. In an example, a wearable frame can be substantially circular (or elliptical), spanning a person's forehead and the rear of the person's head. In an example, a wearable frame can loop around a person's head from one ear to the other.

A mobile wearable electromagnetic brain activity monitor can be used for a variety of purposes. In an example, a mobile wearable electromagnetic brain activity monitor can be used to monitor, measure, and modify a person's food consumption. In an example, a mobile wearable electromagnetic brain activity monitor can be used to control eyewear whose light absorption, light reflection, light refraction, light spectrum transformation, focal direction, focal distance, light polarization, or parallax view is controlled by the wearer's brain activity. In an example, a mobile wearable electromagnetic brain activity monitor can comprise eyeglasses and/or other eyewear.

A person's food consumption has an effect on their electromagnetic brain activity. In an example, data concerning a person's food consumption and data concerning their electromagnetic brain activity can be collected in a chronologically-linked manner. These two data streams (concerning food consumption and brain activity) can be jointly analyzed by a brain activity monitor and/or system in order to identify significant relationships between patterns of food consumption and patterns of brain activity. In an example, this analysis can include a (short) time lag between food consumption and brain activity. In an example, a food-brainwave database can be created, wherein this database links patterns of food consumption with patterns of electromagnetic brain activity.

In an example, data concerning food consumption and data concerning electromagnetic brain activity can be independently collected and jointly analyzed in order to identify significant associations between them. In an example, during a first (calibration) time period, data concerning food consumption can be manually entered via a human-to-computer interface which is part of a wearable EEG monitor. In an example, during a first (calibration) time period, data concerning food consumption can be manually entered into a physically-separate device or a remote computer as part of an overall system of wirelessly-linked devices.

In an example, during a second time period (after calibration), previously-identified associations can be used to estimate a person's food consumption based on their brain activity. In particular, during a second time period, a person's electromagnetic brain activity can be monitored and data concerning their brain activity can be collected. Combining this brain activity data with the associations between food consumption and brain activity which were identified in the first (calibration) period, the person's food consumption can be estimated from the person's brain activity. In an example, data concerning food consumption and brain activity can be analyzed within a data processor which is part of a wearable EEG monitor. In an example, data concerning food consumption and brain activity can be wirelessly transmitted to a physically-separate device or a remote computer and analyzed in that separate device or remote computer as part of an overall system of wirelessly-linked devices.

In an example, a food-brainwave database which links food consumption patterns to brain activity patterns can be created de novo for a specific person. In an example, a previously-created food-brainwave database, which links food consumption patterns to brain activity patterns, can be used and a first (calibration) period can be shortened or skipped entirely. In an example, a general-population food-brainwave database which links food consumption patterns to brain activity patterns can be created for a general population and then tailored, customized, or adapted for use for a specific person. In an example, a general population food-brainwave database can be tailored, customized, or adapted for use for a specific person based on demographic, physiologic, behavioral, health status, geographic and/or environmental parameters for that person. In an example, a general population food-brainwave database can be tailored, customized, or adapted for use for a specific person based on the person's baseline brainwave information. In an example, such tailoring, customization, or adaptation can include Bayesian statistical methods, an artificial neural network, evolving algorithms, and/or AI (e.g. machine learning).

In an example, food consumption can be directly measured during a first (calibration) time period by active entry of food information by a person via a human-to-computer interface such as a touchscreen, speech recognition interface, gesture recognition interface, EMG recognition interface, eye movement recognition interface, keypad, buttons, or knobs. In an example, a person can enter information concerning food that they eat via a software application on a portable electronic device using a menu-driven interface with pictures and descriptions of common foods and portions. In an example, food consumption can be directly measured during a first (calibration) time period by automatic tracking of food consumption via a wearable or handheld imaging device. In an example, a person can take pictures of food from different angles for 3D modeling of food volume as well as automatic food identification. In an example, electronically-functional eyewear with imaging capability can automatically track and identify food consumption using pattern recognition and/or gesture recognition. In an example, a person can use a spectroscopic food probe, scanner, or utensil to also collect information concerning the chemical composition of food. In an example, food consumption can also be independently measured by an intraoral chemical-composition sensor.

In an example, a method can not only track a person's food consumption, but also help the person to modify their food consumption to improve their nutrition, weight management, and overall health. In addition to estimating the person's food consumption based on their brain activity, a brain activity monitor and system can also provide feedback to the person in order to prompt the person to modify their food consumption.

In an example, a brain activity monitor and system can provide feedback to a person via a visual, auditory, or tactile computer-to-human interface in order to inform the person of their estimated food consumption and/or to prompt the person to modify their food consumption. In an example, this feedback can be based on the person's cumulative amount of food consumption. In an example, this feedback can be based on the person's consumption of a specific type or amount of food, ingredient, or nutrient. In an example, this feedback can be conveyed through a physically-separate device such as a smart phone, smart watch, smart wrist band, electronically-functional eyewear and/or contact lenses, wearable camera, other wearable device, tablet, desktop, or other remote computer.

In an example, feedback can be conveyed to a person in the form of a written or spoken message that is delivered through a computer-to-human interface. In an example, feedback can be a text message. In an example, an interface for providing feedback can be part of a wearable EEG monitor itself. In an example, feedback can be delivered via a smart watch, a smart phone, or electronically-functional eyewear. In an example, feedback can be in the form of a visual, auditory, or tactile stimulus. In an example, feedback can be a gentle vibration or quiet tone. In an example, feedback can convey information concerning what type and/or amount of food consumption triggered the feedback and provide suggestions concerning how to modify food consumption to ensure proper nutrition.

In an example, feedback can be triggered by a person's consumption of selected types or amounts of foods, nutrients, and/or ingredients as estimated by brain activity data collected by a wearable EEG monitor. In an example, this feedback can be triggered by the person's cumulative consumption of food and/or calories during a selected period of time. In an example, this feedback can encourage the person to modify their food consumption patterns to achieve better nutrition, proper energy balance, and/or predefined health goals. In an example, this feedback can be part of an overall system for proper nutrition, weight management, and improved health.

In an example, electromagnetic brain activity can be measured by a plurality of electrodes on a mobile electroencephalogram (EEG) monitor which the person wears on their head in an ongoing manner. In an example, electrodes used can be dry electrodes. In an example, a person's brain activity can be measured from one or multiple selected recording sites using a mobile wearable EEG monitor. In an example, measurement of brain activity can comprise measuring electromagnetic data concerning impedance, voltage difference, and/or energy transfer between two sites on a person's head—a selected recording site and a reference site. In an example, electromagnetic brain activity data can be measured by an electrode or other brain activity sensor at a selected recording place. In an example, electromagnetic brain activity data from a selected recording place (relative to a reference place) can be called a channel. In an example, electromagnetic brain activity data from multiple recording places can be called a montage.

In various examples, one or multiple recording places can be selected from the group of EEG placement sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, one or more reference places can be selected from the group of sites consisting of A1 and A2. In an example, brain activity data can be recorded at a rate in the range of 100 to 300 samples per second.

There can be different regions of brain activity for neural processing of the color, shape, texture, odor, taste, and feel of food. In an example, identification of food types and estimates of food quantity can be based on multivariate analysis of the activities of different brain regions associated with visual processing, image recognition, olfactory processing, taste processing, texture processing, and sensory motor processing (such as chewing and swallowing).

In an example, there can be a progressive or phased effect of food consumption on brain activity. In an example, a first phase can occur when a person sees and smells food prior to eating it. In an example, a second phase can occur as a person tastes, smells, and feels food as they eat it. In an example, a third phase can occur as food is digested within a person's gastrointestinal tract and nutrients from food enter the person's blood stream. In an example, statistical analysis of brain activity can comprise analysis of the separate, sequential, or cumulative effects of these three phases of food consumption.

In example, a device or system can comprise a mobile wearable EEG monitor. A person can wear a mobile wearable EEG monitor on an ongoing and ambulatory basis. This EEG monitor can measure their brain activity, via one or more EM data channels during different activities, including eating. In an example, a device or system can further comprise the use of one or more statistical methods to identify associations between: (a) the occurrence of hunger, satiation, a pleasant taste or odor in general, an unpleasant taste or odor in general, a specific taste, a specific odor, food consumption in general, consumption of a specific type of food, consumption of a specific type of nutrient, or consumption of a specific type of ingredient; and (b) the occurrence of a specific pattern of brain activity which is measured from one or more data channels on a mobile wearable EEG monitor.

In an example, these associations can be used to create a food-brainwave database (or library) which links consumption of specific types and/or amounts of foods, nutrients, or ingredients with particular patterns of brain activity. In an example, this database of links between types of food consumption and types of brain activity can be generally applicable for all people. In an example, this database of links between types of food consumption and types of brain activity can be specifically-developed and/or customized for a particular person. In an example, a first database can link consumption of specific types and amounts of food to specific patterns of brain activity and a second database can link specific types and amounts of food to specific types and amounts of nutrients. In an example, sequential use of both databases can provide information on the types and amounts of nutrients which a person consumes based on information concerning their brain activity. In an alternative example, a single database can directly link specific patterns of brain activity and specific types and amounts of nutrients.

In an example, identified associations between food consumption and brain activity can be used to track a person's consumption of foods, nutrients, and/or ingredients. In an example, these associations between food consumption and brain activity can be used to provide feedback to the person concerning their consumption of foods, nutrients, and/or ingredients. In an example, these associations between food consumption and brain activity can be used to provide feedback to the person concerning their brain activity related to consumption of foods, nutrients, and/or ingredients. In an example, these associations between food consumption and brain activity can be used to modify the person's consumption of foods, nutrients, and/or ingredients.

In an example, brain activity can be associated with food consumption during a first (calibration and/or database-creation) time period. In an example, during this calibration period, a database or data library can be created in which statistical models and parameters for linking brain activity to food consumption are estimated. These statistical models and parameters can be estimated for a general population or can be specific to a particular person. In an example, statistical models or parameters can be first estimated for the general population and then customized or adapted to a specific person, optionally through the use of Bayesian statistical methods. In an example, a general database, model, or model parameter can be customized, adjusted, modified, tailored, or adapted for a specific person. In an example, application of a database, model, or model parameter can control for variables selected from the group consisting of: a person's age, a person's gender, a person's health status, the time of day, level of recent physical activity, geographic location, and environmental variables. In an example, a database, model, and model parameters can be estimated de novo for a specific person.

During a calibration period, data concerning food consumption and data concerning brain activity can be collected in a chronologically-linked manner. Data from these two sources can then be jointly analyzed using statistical methods in order to identify significant associations between specific patterns of food consumption and specific patterns of brain activity. In an example, these data streams can be analyzed within the data processor of a wearable EEG monitor. In an example, these data streams can be transmitted wirelessly to a separate device or remote location wherein the data is analyzed. In an example, a calibration or database-creation process can be an iterative one—employing Bayesian statistics, adaptive algorithms, and/or an artificial neural network. In an example, a general database or statistical method that links food consumption and brain activity patterns can be created for the general population and then customized for a specific person. In an example, a database or statistical method that links food consumption and brain activity patterns can be created de novo for a specific person.

In an example, estimation of models and parameters for associating specific patterns of brain activity with specific types of food consumption can be an iterative or evolving process employing AI (e.g. machine learning) and/or an artificial neural network. In an example, a database can link consumption of specific foods, ingredients, or nutrients with specific patterns of EM brain activity. In an example, this linkage can be dependent on, or control for, a variety of control parameters including: a person's age and gender, time of day, activity level, location, etc. In an example, such a database can include brainwave patterns associated with common foods, portion sizes which are commonly associated with these foods, ingredients which are commonly associated with these foods, nutrients which are commonly associated with these foods, and calories which are commonly associated with these foods.

In an example, information concerning food consumption can be collected by a means other than collection of information on brain activity during a first (calibration and/or database-creation) period. In an example, during a calibration and/or database-creation period, information concerning food consumption can be manually entered and/or collected by the person wearing the EEG monitor. In an example, during a calibration and/or database-creation period, information concerning food consumption can be automatically collected by a mechanism and/or device other than measurement of brain activity by the wearable EEG monitor. In an example, during a calibration and/or database creation period, information concerning food consumption can be collected by an interactive combination of automatic data collection and manual data entry.

In an example, during a first (calibration) time period, a person can manually enter information concerning food consumption via a hand-held mobile device such as a smart phone, electronic pad, or electronic tablet. In an example, a person can manually enter information concerning food consumption via a wearable device such as a smart watch, smart bracelet, or electronically-functional eyewear. In an example, a person can manually enter information concerning food consumption via a laptop, desktop, or other relatively fixed-location computer. In an example, a person can manually enter information concerning food consumption via a touchscreen, keyboard, keypad, touch buttons, or gesture recognition interface. In an example, food consumption information can be entered and/or collected via an eye movement detector or EMG sensor. In an example, software for entry of food consumption information can include pictures and descriptions of common food items. In an example, a person can manually enter information concerning food consumption via a speech recognition interface.

In an example, during a first (calibration) time period, a person can actively collect information concerning food consumption by taking one or more pictures of food. In an example, such picture taking can be done with a handheld device which is manually aimed toward food and manually triggered to take pictures. In an example, images of food can be automatically collected by an automatic imaging device which a person wears. In an example, images of food consumed can be automatically collected via a wearable camera or by eyewear with automatic imaging functionality. In an example, manually or automatically obtained pictures of food from different angles can be analyzed using three-dimensional modeling to estimate the volume of food consumed. In an example, a camera or other imaging device can take simultaneous pictures of food from different angles. In an example, a camera or other imaging device can take sequential pictures of food from different angles due to movement of the device, movement of the food, or both such movements. In an example, food consumption can be estimated by a wearable camera or electronically-functional eyewear based on gesture recognition as the person's hands interact with food and their mouth.

In an example, food type can be identified from a picture of food using one or more methods selected from the group consisting of: analysis of food color, shape, and texture; packaging logo and/or label recognition or identification; and bar code recognition or identification. In an example, information concerning food consumed can be collected by scanning a bar code or other digital code associated with a food product sold in a store or menu item in a restaurant. In an example, location as detected by a GPS unit can be a factor in food identification, especially if the location is a restaurant that serves standardized servings and/or sells standardized packages of food.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a GPS unit. In an example, a control unit can comprise a data processor.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a GPS unit; and a database which includes associations between specific patterns of food consumption and specific patterns of electromagnetic brain activity, which translates specific types and amounts of food into specific types and amounts of nutrients, or which does both.

In an example, information concerning types and amounts of food consumed can be collected from a smart food utensil, food probe, or food scanner that analyses the composition and volume of food consumed. In an example, food composition can be automatically analyzed via spectroscopy. In an example, information concerning specific types and amounts of food consumed can be collected during a calibration and/or database-creation period and this information can then be automatically linked to specific types and amounts of nutrients and/or ingredients using a database of common foods, their ingredients, and their nutritional composition. In an example, a hand-held or wearable spectroscopy device can be used to provide independent data concerning the types and amounts of nutrients and/or ingredients in specific types and amounts of food. In an example, a spectroscopy device can analyze the spectrum of light reflected from the surface of food or passing through a layer of food.

In an example, electromagnetic data concerning brain activity can be filtered to remove artifacts before the application of primary statistical methods. In an example, electromagnetic signals from eye blinks, eye flutters, or other eye movements can be removed prior to the application of primary statistical methods. In an example, a notch filter can be used as well to remove 60 Hz artifacts caused by AC electrical lines. In various examples, one or more data filters can be selected from the group consisting of: a high-pass filter, a band-pass filter, a loss-pass filter, an electromyographic activity filter, a 0.5-1 Hz filter, and a 35-70 Hz filter. In an example, a specific pattern of brain activity can follow a specific pattern of food consumption after a time lag. In an example, this time lag can be in the range of 20-100 milliseconds.

In an example, a specific pattern of brain activity associated with a specific pattern of food consumption can be analyzed as an Event Related Potential (ERP). In an example, a specific pattern of food consumption can be associated with a transient pattern of brain activity which does not repeat over time. In an example, a specific pattern of food consumption can be associated with a rhythmic pattern of brain activity which does repeat over time. In an example, statistical methods used to associate specific brainwave patterns with the consumption of specific types and/or amounts of food, ingredients, or nutrients can include analysis of wave frequency, wave frequency band, wave amplitude, wave phase, and wave form or morphology. In an example, wave form or morphology can be identified from the group consisting of: simple sinusoidal wave, composite sinusoidal wave, simple saw-tooth wave, composite saw-tooth wave, biphasic wave, tri-phasic wave, and spike.

During a first (calibration and/or database creation) time period, one or more primary statistical methods can be used to identify significant associations between patterns of food consumption and patterns of brain activity. In an example, a statistical method can comprise finding the mean or average value of data from one or more brain activity channels during a period of time. In an example, a statistical method can comprise identifying a significant change in the mean or average value of data from one or more brain activity channels during or after food consumption. In an example, a statistical method can comprise finding the median value of data from one or more brain activity channels during a period of time. In an example, a statistical method can comprise identifying a significant change in the median value of data from one or more brain activity channels during or after food consumption. In an example, a statistical method can comprise identifying significant changes in the relative mean or median data values among multiple brain activity channels during or after food consumption. In an example, a statistical method can comprise identifying significant changes in mean data values from a first set of electrode locations relative to mean data values from a second set of electrode locations during or after food consumption. In an example, a statistical method can comprise identifying significant changes in mean data recorded from a first region of the brain relative to mean data recorded from a second region of the brain during or after food consumption.

In an example, a statistical method can comprise finding the minimum or maximum value of data from one or more brain activity channels during a period of time. In an example, a statistical method can comprise identifying a significant change in the minimum or maximum value of data from one or more brain activity channels during or after food consumption. In an example, a statistical method can comprise identifying significant changes in the relative minimum or maximum data values among multiple brain activity channels during or after food consumption. In an example, a statistical method can comprise identifying significant changes in minimum or maximum data values from a first set of electrode locations relative to minimum or maximum data values from a second set of electrode locations during or after food consumption. In an example, a statistical method can comprise identifying significant changes in minimum or maximum data values recorded from a first region of the brain relative to minimum or maximum data values recorded from a second region of the brain during or after food consumption.

In an example, a statistical method can comprise finding the variance or the standard deviation of data from one or more brain activity channels during a period of time. In an example, a statistical method can comprise identifying a significant change in the variance or the standard deviation of data from one or more brain activity channels during or after food consumption. In an example, a statistical method can comprise identifying significant changes in the covariation and/or correlation among data from multiple brain activity channels during or after food consumption. In an example, a statistical method can comprise identifying significant changes in the covariation or correlation between data from a first set of electrode locations relative and data from a second set of electrode locations during or after food consumption. In an example, a statistical method can comprise identifying significant changes in the covariation or correlation of data values recorded from a first region of the brain and a second region of the brain during or after food consumption.

In an example, a statistical method can comprise finding the mean amplitude of waveform data from one or more channels during a period of time. In an example, a statistical method can comprise identifying a significant change in the mean amplitude of waveform data from one or more channels during or after food consumption. In an example, a statistical method can comprise identifying significant changes in the relative means of wave amplitudes from one or more channels during or after food consumption. In an example, a statistical method can comprise identifying significant changes in the amplitude of EM signals recorded from a first region of the brain relative to the amplitude of EM signals recorded from a second region of the brain during or after food consumption.

In an example, a statistical method can comprise finding the power of waveform brain activity data from one or more channels during a period of time. In an example, a statistical method can comprise identifying a significant change in the power of waveform data from one or more channels during or after food consumption. In an example, a statistical method can comprise identifying significant changes in the relative power levels of one or more channels during or after food consumption. In an example, a statistical method can comprise identifying significant changes in the power of EM signals recorded from a first region of the brain relative to the power of EM signals recorded from a second region of the brain during or after food consumption.

In an example, a statistical method can comprise finding a frequency or frequency band of waveform and/or rhythmic brain activity data from one or more channels which repeats over time. In an example, Fourier Transform methods can be used to find a frequency or frequency band of waveform and/or rhythmic data which repeats over time. In an example, a statistical method can comprise decomposing a complex waveform into a combination of simpler waveforms which each repeat at a different frequency or within a different frequency band. In an example, Fourier Transform methods can be used to decomposing a complex waveform into a combination of simpler waveforms which each repeat at a different frequency or within a different frequency band.

In an example, a statistical method can comprise identifying significant changes in the amplitude, power level, phase, frequency, and/or oscillation of waveform data from one or more channels during or after food consumption. In an example, a statistical method can comprise identifying significant changes in the amplitude, power level, phase, frequency, and/or oscillation of waveform data within a selected frequency band during or after food consumption. In an example, a statistical method can comprise identifying significant changes in the relative amplitudes, power levels, phases, frequencies, and/or oscillations of waveform data among different frequency bands during or after food consumption. In various examples, these significant changes can be identified using Fourier Transform methods.

In an example, brainwaves (or other rhythmic, cyclical, and/or repeating electromagnetic signals associated with brain activity) can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier Transformation methods. In an example, brainwaves can be measured and analyzed using five common clinical frequency bands—Delta, Theta, Alpha, Beta, and Gamma. In an example, changes in brainwaves in a single frequency band, changes in brainwaves in multiple frequency bands, or changes in brainwaves in a first frequency band relative to those in a second frequency band can be associated with changes in food consumption. These associations can be used, in turn, to track and modify food consumption.

In an example, Delta brainwaves can be measured and analyzed within a selected frequency band. In an example, Delta brainwaves can be measured and analyzed within the frequency band of 1 to 4 Hz. In various examples, Delta brainwaves (or other rhythmic, cyclical, and/or repeating electromagnetic signals associated with brain activity) can be measured and analyzed within a frequency band selected from the group consisting of: 0.5-3.5 Hz, 0.5-4 Hz, 1-3 Hz, 1-4 Hz, and 2-4 Hz. In an example, changes in Delta brainwaves can be identified and associated with changes in food consumption in order to track and/or modify food consumption. In an example, specific patterns or trends in brainwaves in the Delta frequency band can be statistically associated with consumption of specific amounts and types of foods, ingredients, and/or nutrients. These statistical associations can be used to track an individual's cumulative consumption of specific amounts of these of foods, ingredients, and/or nutrients during a period of time. These statistical associations can also be used to provide feedback to an individual in order to modify their consumption of these foods, ingredients, and/or nutrients.

In an example, hunger or satiety can be associated with a change in the power of brainwaves in the Delta frequency band. In an example, hunger can be associated with a decrease in the relative power of brainwaves in the Delta band. In an example, satiety can be associated with an increase in the relative power of brainwaves in the Delta band. In an example, hunger or satiety can be associated with a frequency shift within the Delta frequency band. In an example, hunger can be associated with an upward shift in the frequency of brainwaves within the Delta band. In an example, satiety can be associated with a downward shift in the frequency of brainwaves within the Delta band.

In an example, hunger or satiety can be associated with a change in wave shape for brainwaves in the Delta frequency band. In an example, hunger or satiety can be associated with a change in which brain regions originate or modify brainwaves within the Delta frequency band. In an example, hunger or satiety can be associated with a change in brainwave activity within the Delta band from the anterior vs. posterior a person's brain. In an example, hunger or satiety can be associated with a change in brainwave activity within the Delta band for a particular brain lobe or organelle. In an example, hunger or satiety can be associated with a change in brainwave activity within the Delta band as measured from a specific electrode site, a specific electrode channel, and/or a specific montage of channels.

In an example, there can be changes in the power of brainwaves in the Delta frequency band during food consumption in general. In an example, food consumption can be associated with a change in the relative power of brainwaves in the Delta band. In an example, food consumption can be associated with a frequency shift in brainwaves within the Delta frequency band. In an example, food consumption can be associated with a change in wave shape for brainwaves in the Delta frequency band. In an example, food consumption can be associated with a change in which brain regions originate or modify brainwaves within the Delta frequency band. In an example, food consumption can be associated with a change in brainwave activity within the Delta band as measured from a specific electrode site, a specific electrode channel, and/or a specific montage of channels.

In an example, pleasant or unpleasant tastes and/or odors can be associated with changes in the power of brainwaves in the Delta frequency band. In an example, pleasant tastes and/or odors can be associated with increases in the relative power of brainwaves in the Delta band. In an example, unpleasant tastes and/or odors can be associated with decreases in the relative power of brainwaves in the Delta band. In an example, pleasant or unpleasant tastes and/or odors can be associated with shifts in the frequency of brainwaves within the Delta frequency band. In an example, pleasant tastes and/or odors can cause an upward shift in brainwave frequency within the Delta band. In an example, unpleasant tastes and/or odors can cause a downward shift in brainwave frequency within the Delta band. In an example, pleasant or unpleasant tastes or odors can be associated with changes in wave shape for brainwaves in the Delta frequency band. In an example, pleasant or unpleasant tastes or odors can be associated with changes in which brain regions originate or modify brainwaves within the Delta frequency band. In an example, pleasant or unpleasant tastes or odors can be associated with changes in brainwave activity within the Delta band as measured from a specific electrode site, a specific electrode channel, and/or a specific montage of channels.

In an example, specific tastes and/or odors can cause specific changes in the power, frequency, oscillation, wave shape, coherence, and/or brain region origins of brainwaves in the Delta frequency band. In an example, consumption of specific types of food, nutrients, and/or ingredients can cause specific changes in the power, frequency, oscillation, wave shape, coherence, and/or brain region origins of brainwaves in the Delta frequency band. In an example, changes in the power, frequency, oscillation, wave shape, coherence, and/or brain region origins of brainwaves in the Delta band can be caused by a person's consumption of foods, ingredients, and/or nutrients selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food.

In an example, Theta brainwaves can be measured and analyzed within a selected frequency band. In an example, Theta brainwaves can be measured and analyzed within the frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz. In an example, changes in Theta brainwaves can be identified and associated with changes in food consumption in order to track and/or modify food consumption. In an example, specific patterns or trends in brainwaves in the Theta frequency band can be statistically associated with consumption of specific amounts and types of foods, ingredients, and/or nutrients. These statistical associations can be used to track an individual's cumulative consumption of specific amounts of these of foods, ingredients, and/or nutrients during a period of time. These statistical associations can also be used to provide feedback to an individual in order to modify their consumption of these foods, ingredients, and/or nutrients.

In an example, hunger or satiety can be associated with a change in the power of brainwaves in the Theta frequency band. In an example, hunger can be associated with an increase in the relative power of brainwaves in the Theta band. In an example, hunger can be associated with a frequency shift within the Theta frequency band. In an example, hunger or satiety can be associated with changes in wave shape for brainwaves in the Theta frequency band. In an example, hunger or satiety can be associated with a change in which brain regions originate or modify brainwaves within the Theta frequency band. In an example, hunger or satiety can be associated with a change in brainwave activity within the Theta band as measured from a specific electrode site, a specific electrode channel, and/or a specific montage of channels.

In an example, there can be changes in the power of brainwaves in the Theta frequency band during food consumption in general. In an example, food consumption can be associated with a change in the relative power of brainwaves in the Theta band. In an example, food consumption can be associated with a frequency shift in brainwaves within the Theta frequency band. In an example, food consumption can be associated with a change in wave shape for brainwaves in the Theta frequency band. In an example, food consumption can be associated with a change in which brain regions originate or modify brainwaves within the Theta frequency band. In an example, food consumption can be associated with a change in brainwave activity within the Theta band as measured from a specific electrode site, a specific electrode channel, and/or a specific montage of channels.

In an example, pleasant or unpleasant tastes and/or odors can be associated with changes in the power of brainwaves in the Theta frequency band. In an example, pleasant tastes and/or odors can be associated with a decrease in the relative power of brainwaves in the Theta band. In an example, pleasant or unpleasant tastes and/or odors can be associated with shifts in the frequency of brainwaves within the Theta frequency band. In an example, pleasant or unpleasant tastes or odors can be associated with changes in wave shape for brainwaves in the Theta frequency band. In an example, pleasant or unpleasant tastes or odors can be associated with changes in which brain regions originate or modify brainwaves within the Theta frequency band. In an example, pleasant or unpleasant tastes or odors can be associated with changes in brainwave activity within the Theta band as measured from a specific electrode site, a specific electrode channel, and/or a specific montage of channels.

In an example, Alpha brainwaves can be measured and analyzed within a selected frequency band. In an example, Alpha brainwaves can be measured and analyzed within the frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, changes in Alpha brainwaves can be identified and associated with changes in food consumption in order to track and/or modify food consumption In an example, specific patterns or trends in brainwaves in the Alpha frequency band can be statistically associated with consumption of specific amounts and types of foods, ingredients, and/or nutrients. These statistical associations can be used to track an individual's cumulative consumption of specific amounts of these of foods, ingredients, and/or nutrients during a period of time. These statistical associations can also be used to provide feedback to an individual in order to modify their consumption of these foods, ingredients, and/or nutrients.

In an example, hunger or satiety can be associated with a change in the power of brainwaves in the Alpha frequency band. In an example, hunger can be associated with an increase in the relative power of brainwaves in the Alpha band. In an example, satiety can be associated with a decrease in the relative power of brainwaves in the Alpha band. In an example, hunger or satiety can be associated with a frequency shift within the Alpha frequency band. In an example, hunger can be associated with a downward shift in the frequency of brainwaves within the Alpha band. In an example, satiety can be associated with an upward shift in the frequency of brainwaves within the Alpha band. In an example, hunger or satiety can be associated with a change in wave shape for brainwaves in the Alpha frequency band. In an example, hunger or satiety can be associated with a change in which brain regions originate or modify brainwaves within the Alpha frequency band. In an example, hunger or satiety can be associated with a change in brainwave activity within the Alpha band on one side of a person's brain relative to the other side. In an example, hunger or satiety can be associated with a change in brainwave activity within the Alpha band in a particular brain lobe or organelle. In an example, hunger or satiety can be associated with a change in brainwave activity within the Alpha band as measured from a specific electrode site, a specific electrode channel, and/or a specific montage of channels.

In an example, there can be changes in the power of brainwaves in the Alpha frequency band during food consumption in general. In an example, food consumption can be associated with a change in the relative power of brainwaves in the Alpha band. In an example, food consumption can be associated with a frequency shift in brainwaves within the Alpha frequency band. In an example, food consumption can be associated with a change in wave shape for brainwaves in the Alpha frequency band. In an example, food consumption can be associated with a change in which brain regions originate or modify brainwaves within the Alpha frequency band. In an example, food consumption can be associated with a change in brainwave activity within the Alpha band as measured from a specific electrode site, a specific electrode channel, and/or a specific montage of channels.

In an example, pleasant or unpleasant tastes and/or odors can be associated with changes in the power of brainwaves in the Alpha frequency band. In an example, pleasant tastes and/or odors can be associated with increases in the relative power of brainwaves in the Alpha band. In an example, unpleasant tastes and/or odors can be associated with decreases in the relative power of brainwaves in the Alpha band. In an example, pleasant or unpleasant tastes and/or odors can be associated with shifts in the frequency of brainwaves within the Alpha frequency band. In an example, pleasant tastes and/or odors can cause an upward shift in brainwave frequency within the Alpha band. In an example, unpleasant tastes and/or odors can cause a downward shift in brainwave frequency within the Alpha band. In an example, pleasant or unpleasant tastes or odors can be associated with changes in wave shape for brainwaves in the Alpha frequency band. In an example, pleasant or unpleasant tastes or odors can be associated with changes in which brain regions originate or modify brainwaves within the Alpha frequency band. In an example, pleasant or unpleasant tastes or odors can be associated with changes in brainwave activity within the Alpha band as measured from a specific electrode site, a specific electrode channel, and/or a specific montage of channels.

In an example, specific tastes and/or odors can cause specific changes in the power, frequency, oscillation, wave shape, coherence, and/or brain region origins of brainwaves in the Alpha frequency band. In an example, consumption of specific types of food, nutrients, and/or ingredients can cause specific changes in the power, frequency, oscillation, wave shape, coherence, and/or brain region origins of brainwaves in the Alpha frequency band. In an example, changes in the power, frequency, oscillation, wave shape, coherence, and/or brain region origins of brainwaves in the Alpha band can be caused by a person's consumption of foods, ingredients, and/or nutrients selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food.

In an example, Beta brainwaves can be measured and analyzed within a selected frequency band. In an example, Beta brainwaves can be measured and analyzed within the frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, changes in Beta brainwaves can be identified and associated with changes in food consumption in order to track and/or modify food consumption. In an example, specific patterns or trends in brainwaves in the Beta frequency band can be statistically associated with consumption of specific amounts and types of foods, ingredients, and/or nutrients. These statistical associations can be used to track an individual's cumulative consumption of specific amounts of these of foods, ingredients, and/or nutrients during a period of time. These statistical associations can also be used to provide feedback to an individual in order to modify their consumption of these foods, ingredients, and/or nutrients.

In an example, Gamma brainwaves can be measured and analyzed within a selected frequency band. In an example, Gamma brainwaves can be measured and analyzed within the frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz. In an example, changes in Gamma brainwaves can be identified and associated with changes in food consumption in order to track and/or modify food consumption. In an example, specific patterns or trends in brainwaves in the Gamma frequency band can be statistically associated with consumption of specific amounts and types of foods, ingredients, and/or nutrients. These statistical associations can be used to track an individual's cumulative consumption of specific amounts of these of foods, ingredients, and/or nutrients during a period of time. These statistical associations can also be used to provide feedback to an individual in order to modify their consumption of these foods, ingredients, and/or nutrients.

In an example, multivariate analysis of brainwave activity in the Delta, Theta, and Alpha frequency bands can identify patterns which are associated with: hunger, satiety, food consumption in general, pleasant tastes or odors in general, unpleasant tastes or odors in general, specific tastes or odors, and/or consumption of specific types of foods, nutrients, or ingredients. These statistical associations can be used to track an individual's cumulative consumption of amounts and types of foods, ingredients, and/or nutrients. These statistical associations can also be used to provide feedback to an individual in order to modify their consumption of these foods, ingredients, and/or nutrients.

In various examples, multivariate analysis of brainwave activity in two or more frequency bands selected from the group consisting of Delta, Theta, Alpha, Beta, and Gamma can identify patterns which are associated with: hunger, satiety, food consumption in general, pleasant tastes or odors in general, unpleasant tastes or odors in general, specific tastes or odors, and/or consumption of specific types of foods, nutrients, or ingredients. In an example, correlation and/or covariance analysis of brainwave activity in two or more of frequency bands selected from the group consisting of Delta, Theta, Alpha, Beta, and Gamma can identify patterns which are associated with: hunger, satiety, food consumption in general, pleasant tastes or odors in general, unpleasant tastes or odors in general, specific tastes or odors, and/or consumption of specific types of foods, nutrients, or ingredients. In an example, multivariate discriminant analysis of brainwave activity in two or more of frequency bands selected from the group consisting of Delta, Theta, Alpha, Beta, and Gamma can identify patterns which are associated with: hunger, satiety, food consumption in general, pleasant tastes or odors in general, unpleasant tastes or odors in general, specific tastes or odors, and/or consumption of specific types of foods, nutrients, or ingredients. These statistical associations can be used to track an individual's cumulative consumption of amounts and types of foods, ingredients, and/or nutrients. These statistical associations can also be used to provide feedback to an individual in order to modify their consumption of these foods, ingredients, and/or nutrients.

In various examples, specific patterns of brain activity can be associated with consumption of specific types and/or amounts of food, ingredients, and/or nutrients using one or more statistical methods selected from the group consisting of: ANOVA or MANOVA; artificial neural network; auto-regression; Bonferroni analysis; centroid analysis; chi-squared analysis; cluster analysis and grouping; decision tree or random forest analysis; Discrete Fourier transform (DFT), Fast Fourier Transform (FFT), or other Fourier Transform methods; factor analysis; feature vector analysis; fuzzy logic model; Gaussian model; hidden Markov model, input-output hidden Markov model, or other Markov model; inter-band mean; inter-band ratio; inter-channel mean; inter-channel ratio; inter-montage mean; inter-montage ratio; Kalman filter; kernel estimation; linear discriminant analysis; linear transform; logit model; AI (e.g. machine learning); mean power; mean; median; multi-band covariance analysis; multi-channel covariance analysis; multivariate linear regression or multivariate least squares estimation; multivariate logit or other multivariate parametric classifiers; naive Bayes classifier, trained Bayes classifier, dynamic Bayesian network, or other Bayesian methods; non-linear programming; pattern recognition; power spectral density or other power spectrum analysis; principal components analysis; probit model; support vector machine; time-series model; T-test; variance, covariance, or correlation; waveform identification; multi-resolution wavelet analysis or other wavelet analysis; whole band power; and Z-scores or other data normalization method.

In an example, after a first (calibration) time period is completed, a device or system can be used to track a person's food consumption by measuring and analyzing their brain activity using a wearable EEG monitor. In an example, food consumption can be automatically detected by measurement and analysis of brain activity. In an example, consumption of specific types of foods, ingredients, and nutrients can be automatically monitored, detected, and measured by a wearable EEG monitor using selected statistical methods, model parameters, and/or databases linking food consumption patterns to brain activity patterns. In an example, such a database can include brainwave patterns associated with common foods, portion sizes which are commonly associated with these foods, ingredients which are commonly associated with those foods, nutrients which are commonly associated with these foods, and calories which are commonly associated with these foods.

In an example, automatic monitoring of food consumption based on brain activity can make use of statistical methods, model parameters, and/or databases which were identified and created during an earlier calibration period. In an example, a person's consumption of selected foods, ingredients, and/or nutrients can also be modified through feedback based on these statistical methods, model parameters, and/or databases. In an example, feedback can help a person to eat less unhealthy food and/or eat more healthy food. In an example, feedback can help a person to better balance their caloric intake and caloric expenditure to better manage their weight. In an example, automatic monitoring and measurement of food consumption via a wearable EEG monitor can be supplemented or refined by other methods of monitoring and measuring food consumption.

In an example, a wearable EEG monitor to track brain activity can be used to estimate the wearer's consumption of selected types and amounts of foods, ingredients, and nutrients. In an example, this tracking of food consumption can be based on identification of brain activity patterns which have been previously-associated with consumption of specific types and amounts of foods, ingredients, and nutrients. In an example, food consumption can be broadly defined to include consumption of liquid beverages and gelatinous food as well as solid food.

In an example, a method for identifying associations between a person's food consumption and their electromagnetic brain activity can comprise: (a) receiving data concerning a person's food consumption from a selected time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the selected time period from one or more electrodes or other brain activity sensors which are configured to be less than one inch from the surface of the person's head; and (c) identifying associations between patterns of food consumption and patterns of electromagnetic brain activity by analyzing data concerning the person's food consumption and data concerning the person's electromagnetic brain activity from the selected time period.

In an example, a method for creating a food-to-brainwave database of associations between a person's food consumption and their electromagnetic brain activity can comprise: (a) receiving data concerning a person's food consumption from a selected time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the selected time period from one or more electrodes or other brain activity sensors which are configured to be less than one inch from the surface of the person's head; and (c) creating a food-brainwave database, wherein this database links patterns of food consumption to patterns of electromagnetic brain activity, by analyzing data concerning the person's food consumption and data concerning the person's electromagnetic brain activity from the selected time period.

In an example, a method for measuring a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) using associations between patterns of food consumption and patterns of electromagnetic brain activity in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients.

In an example, a method for measuring a person's food consumption can comprise: (a) receiving data concerning a person's electromagnetic brain activity from a selected time period from one or more electrodes or other brain activity sensors which are configured to be less than one inch from the surface of the person's head; and (b) using associations between patterns of food consumption and patterns of electromagnetic brain activity in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients.

In an example, a method for measuring a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) using the food-brainwave database, wherein this database links patterns of food consumption to patterns of electromagnetic brain activity, in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients.

In an example, a method for measuring a person's food consumption can comprise: (a) receiving data concerning a person's electromagnetic brain activity from a selected period from one or more electrodes or other brain activity sensors which are configured to be less than one inch from the surface of the person's head; and (b) using a food-brainwave database, wherein this database links patterns of food consumption to patterns of electromagnetic brain activity, in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using associations between patterns of food consumption and patterns of electromagnetic brain activity in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption during the second time period in order to prompt the person to modify their food consumption.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using associations between patterns of food consumption and patterns of electromagnetic brain activity in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption during the selected time period in order to prompt the person to modify their food consumption.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using the food-brainwave database, wherein this database links patterns of food consumption to patterns of electromagnetic brain activity, in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption during the second time period in order to prompt the person to modify their food consumption.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using a food-brainwave database, wherein this database links patterns of food consumption to patterns of electromagnetic brain activity, in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption during the selected time period in order to prompt the person to modify their food consumption.

In an example, a method, device, or system to measure and/or modify a person's food consumption can use a wearable electroencephalogram (EEG) monitor, wherein modification of food consumed is achieved by self-modification of brain activity by the person wearing the monitor.

In an example, a person wearing an EEG monitor can receive feedback concerning their brain activity. In an example, this feedback can be biofeedback. In an example, a visual, auditory, or tactile computer interface can provide a person with feedback concerning their brain activity patterns and helps the person to self-modify these brain activity patterns as a means of modifying their food consumption. In an example, the feedback provided can help the person to self-modify their brain activity patterns in order to self-modify their desire for selected types or amounts of food.

In an example, modification of food consumption can be mediated through self-modification of a person's brain activity. In an example, interactive feedback concerning brain activity patterns can help a person to modify their brain activity patterns into brain activity patterns that are associated with feelings of satiety. In an example, self-modification of brain activity patterns into patterns that are associated with feelings of satiety can help a person to reduce their food consumption. In an example, interactive feedback concerning brain activity patterns can help a person to modify their brain activity patterns into brain activity patterns that are associated with consumption of food that they like. In an example, self-modification of brain activity patterns into patterns that are associated with consumption of food that they like can help a person to increase their consumption of healthy food that they normally dislike. In an example, such feedback can also be useful in helping a person to exercise self-control with respect to addictive behavior.

In an example, a computer interface which helps a person to self-modify their brain activity can comprise an interactive graphical display on a computer screen. In an example, this interactive graphical display can change from a first display configuration to a second display configuration as the person's brain activity changes from a first EM pattern to a second EM pattern. In an example, the second EM pattern of brain activity can be associated with a feeling of satiety. In an example, this second pattern of brain activity can be associated with consumption of a food that the person likes. In an example, the person can change their brain activity (to modify their food consumption) by concentrating on shifting the interactive graphic display from a first display configuration to a second display configuration. In an example, an interactive graphical display can be a changing geometric pattern, color pattern, moving bar, or moving dial. In an example, an interactive graphical display can be a face with a changing expression.

In an example, a computer interface which helps a person to self-modify their brain activity can comprise an interactive audio signal. In an example, this interactive audio signal can change from a first sound pattern to a second sound pattern as the person's brain activity changes from a first EM pattern to a second EM pattern. In an example, the second EM pattern of brain activity can be associated with a feeling of satiety. In an example, this second pattern of brain activity can be associated with consumption of a food that the person likes. In an example, the person can change their brain activity (to modify their food consumption) by concentrating on shifting the interactive audio signal from a first sound pattern to a second sound pattern. In an example, an interactive audio signal can be a changing tone or a changing blend of tones. In an example, an interactive audio signal can be music with changing parameters.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using associations between patterns of food consumption and patterns of electromagnetic brain activity in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption and their electromagnetic brain activity from the second time period in order to prompt the person to modify their food consumption by self-modifying their electromagnetic brain activity.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using associations between patterns of food consumption and patterns of electromagnetic brain activity in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption and their electromagnetic brain activity from the selected time period in order to prompt the person to modify their food consumption by self-modifying their electromagnetic brain activity.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using the food-brainwave database, wherein this database links patterns of food consumption to patterns of electromagnetic brain activity, in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption and their electromagnetic brain activity from the second time period in order to prompt the person to modify their food consumption by self-modifying their electromagnetic brain activity.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using a food-brainwave database, wherein this database links patterns of food consumption to patterns of electromagnetic brain activity, in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption and their electromagnetic brain activity from the selected time period in order to prompt the person to modify their food consumption by self-modifying their electromagnetic brain activity.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) providing feedback to the person concerning their electromagnetic brain activity from the second time period in order to prompt the person to modify their food consumption by self-modifying their electromagnetic brain activity.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; and (b) providing feedback to the person concerning their electromagnetic brain activity from the selected time period in order to prompt the person to modify their food consumption by self-modifying their electromagnetic brain activity.

In an example, a method for modifying a person's food consumption can comprise: (a) receiving data concerning a person's food consumption from a first time period, wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) providing feedback to the person concerning their electromagnetic brain activity from the second time period in order to prompt the person to modify their food consumption by self-modifying their electromagnetic brain activity.

In an example, a method, device, or system to measure and/or modify a person's food consumption can use both: (a) direct visual, auditory, and/or tactile feedback which prompts the person to modify their food consumption; and (b) interactive indirect feedback concerning the person's electromagnetic brain activity which prompts the person to self-modify their brain activity in order to modify their food consumption. In an example, both direct feedback and indirect biofeedback can be used.

In an example, a mobile EEG monitor can provide feedback concerning brain activity to a person wearing the monitor and this feedback can help the person to modify their food consumption. In an example, such feedback can enable the person to modify their brain activity which, in turn, modifies their food consumption. In an example, such feedback can be triggered by the person's consumption of selected types or amounts of foods, nutrients, and/or ingredients as measured by brain activity data collected by the mobile EEG monitor. This tracking of food consumption based on brain activity patterns can be based on previously-identified associations between specific types of food consumption and specific patterns of brain activity. In an alternative example, this feedback can be self-initiated by the person in a proactive manner when the person craves an amount or type of food which is unhealthy to consume.

In an example, modification of food consumption can be caused by biofeedback and self-modification of brain activity. In an example, a device or system can provide a computer-user interface which conveys information concerning brain activity patterns to a person wearing a mobile EEG monitor and helps the person to self-modify these brain activity patterns in order to change their desire for, and consumption of, a selected type or amount of food. In an example, a computer-user interface can help the person to self-modify their brain activity pattern toward a pattern that has been shown to be associated with satiety. In an example, this computer-user interface can provide interactive visual, auditory, and/or tactile cues to help the person self-modify their brain activity pattern. In an example, this computer-user interface can provide interactive graphical, avatar, or musical cues to help the person self-modify their brain activity. In an example, this computer-user interface can help the person to modify their brain activity pattern from a first pattern that is associated with hunger to a second pattern that is associated with satiety. In an example, self-modification of brain activity toward satiety can help the person to limit overeating.

In an example, a computer-user interface can help a person to self-modify their brain activity pattern toward a pattern that has been shown to be associated with a good-tasting food. In an example, this computer-user interface can employ visual, auditory, and/or tactile cues to help the person self-modify their brain activity pattern toward an activity pattern that is associated with good-tasting food. In an example, this computer-user interface can help the person to modify their brain activity pattern from a first pattern that is associated with a first type or amount of food to a second pattern that is associated with a second type or amount of food. In an example, self-modification of brain activity toward a pattern that is associated with consuming a less-healthy (but more-appealing) type of food can help the person to instead consume a more-healthy (but less-appealing) type of food. In an example, with practice and help from a device, a person can train their brain to respond more favorably to healthy food and less favorably to unhealthy food. In an example, a device can help a person to exercise mind over platter.

In an example, a device (or in a system of wirelessly-linked devices) can comprise hardware for the operation of the above-discussed methods of food consumption measurement and modification. In an example, a device which is used for measuring and/or modifying a person's food consumption can include a mobile wearable EEG monitor. In an example, a wearable EEG monitor can further comprise one or more components selected from the group consisting of: one or more electrodes or other brain activity sensors; one or more accelerometers; one or more cameras; a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; a data memory component; a data processor; a GPS component; a heart rate monitor; a power source and/or power-transducing component; and a wireless data transmission and data reception component.

In an example, a wearable EEG monitor can comprise a plurality of electrodes and a control unit. In an example, a control unit can comprise one or more components selected from the group consisting of: a power source and/or power-transducing component; a wireless data transmission and data reception component; a data memory component; and a data processor. In an example, a control unit can further comprise one or more components selected from the group consisting of: a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; one or more accelerometers; one or more cameras; and a GPS component.

In various examples, in addition to electrodes which measure brain activity, a wearable EEG monitor can further comprise one or more sensors selected from the group consisting of: accelerometer, inclinometer, gyroscope, strain gauge, or other motion or position sensor; microphone or other sound sensor; thermometer or other temperature sensor; camera or other imaging sensor; optical sensor or optoelectronic sensor; blood pressure sensor; ECG/EKG sensor, heart rate monitor, and/or heart rate sensor; EMG sensor or other muscle activity sensor; GPS sensor, other location sensor, magnetometer, or compass; spectroscopy sensor or other spectral analysis sensor; electrochemical sensor; blood oximetry sensor; piezoelectric sensor; chewing sensor or swallowing sensor; respiration sensor; pressure sensor; galvanic skin response sensor; and taste or odor sensor.

In an example, a power source for a wearable EEG monitor can be selected from the group consisting of: power from a power source that is internal to the device during regular operation (such as an internal battery, capacitor, energy-storing microchip, or wound coil or spring); power that is obtained, harvested, or transduced from a power source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion, electromagnetic energy from the person's body, blood flow or other internal fluid flow, glucose metabolism, or thermal energy from the person's body.

In an example, a wearable EEG monitor can be in data communication with a separate electronic device comprising one or more components selected from the group consisting of: one or more accelerometers; one or more cameras; a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; a data memory component; a data processor; a GPS component; a power source and/or power-transducing component; and a wireless data transmission and data reception component. In an example the combination of a wearable EEG monitor and a separate electronic device can together comprise a system for using brain activity to measure and modify food consumption.

In an example, operation of a device or system can occur within a self-contained wearable EEG monitor. In an example, operation can occur within a distributed system of which a wearable device, such as a mobile wearable EEG monitor, is one component, and wherein the mobile wearable EEG monitor is in (wireless) communication with a remote computing device which is part of the overall system for using brain activity to measure and modify food consumption. In an example, all aspects of human-to-computer interaction and computer-to-human interaction can occur via one or more interfaces which are physical components of a wearable EEG monitor. In an example, some or all aspects of human-to-computer interaction and/or computer-to-human interaction can occur via one or more interfaces in one or more physically-separate remote devices with which a wearable EEG monitor is in wireless data communication. In an example, a person can use a physically-separate device to enter information concerning food consumption during a calibration period. In an example, a person can use a physically-separate device to enter information concerning food consumption when promoted by analysis of brain activity data collected by the wearable EEG monitor.

In an example, a separate computing device with which a wearable EEG monitor communicates can also be worn by the person. In an example, the EEG monitor together with a separate computing device can comprises a system for measuring and/or modifying a person's food consumption. In an example, a separate computing device with which an EEG monitor is in data communication can worn on a person's wrist, hand, finger, arm, waist, torso, legs, neck, ear(s), and/or head. In an example, a separate computing device with which an EEG monitor is in data communication can be attached to a person's clothing by a means selected from the group consisting of: band, strap, clip, clamp, snap, pin, hook and eye fastener, magnet, and adhesive.

In various example, a separate wearable computing device with which a wearable EEG monitor is in data communication can be selected from the group consisting of: a wristwatch, smart watch, fitness watch, watch phone, bracelet phone, smart bracelet, fitness bracelet, smart wrist band, electronically-functional wrist band, other wrist-worn electronic device, or smart armband; smart glasses, smart eyewear, augmented reality eyewear, virtual reality eyewear, an electronically-functional visor, electronically-functional contact lens, or other electronically-functional eyewear; a smart button, electronically-functional button, pin, brooch, pendant, beads, neck chain, necklace, dog tags, locket, or medallion; a smart finger ring, electronically-functional finger ring, electronically-functional earring, nose ring, or ear bud or clip; a wearable camera; an article of smart clothing, an electronically-functional shirt, electronically-functional pants, or a smart belt; electronically-functional headband, hair pin, headphones, or ear phones; electronically-functional dental appliance, dental attachment, palatal vault attachment, or other electronically-functional intraoral device.

In an example, a separate computing device with which the EEG monitor communicates can also be held and/or carried by the person. In an example, a separate hand-held or carried computing device with which an EEG monitor is in data communication can be selected from the group consisting of: smart phone, mobile phone, or cellular phone; PDA; electronic tablet; electronic pad; smart food utensil; and other electronically-functional handheld device. In an example, a separate computing device with which the EEG monitor communicates can in a relatively-fixed remote location. In an example, a separate computing device with which an EEG monitor is in data communication can be selected from the group consisting of: laptop computer, desktop computer, internet terminal, smart appliance, or other fixed-location electronic communication device.

In an example, the locations of electrodes (or other brain activity sensors) on a wearable EEG monitor can be identified according to the International 10-20 System and/or the Modified Combinatorial Nomenclature (MCN). In an example, with the possible exception of reference sites such as A1 and A2, the brain activity monitoring component of a device can comprise a wearable array of nineteen electrodes or other brain activity sensors. In an example, this array can be located substantially at the following placement sites: FP1, FP2, F7, F3, Fz, F4, F8, T3/T7, C3, C4, Cz, T4/T8, T5/P7, P3, Pz, P4, T6/P8, O1, and O2. In an example, with the possible exception of reference sites such as A1 and A2, the brain activity monitoring component of a device can comprise a wearable array of seventeen electrodes or other brain activity sensors. In an example, this array can be located substantially at the following placement sites: F3, F4, F7, F8, Fz, T3, T4, T5, T6, P3, P4, Pz, O1, O2, C3, C4, and Cz. In an example, with the possible exception of reference sites, a device can comprise a wearable array of sixteen electrodes or other brain activity sensors. In an example, a wearable array of brain activity sensors can be located substantially at the following placement sites: F3, Fz, F4, T3/T7, C3, C4, Cz, T4/T8, T5/P7, P3, Pz, P4, T6/P8, PO7, PO8, and Oz. In an example, this array can be placed substantially at the following placement sites: F3, Fz, F4, C3, C1, Cz, C2, C5, T4/T8, CPz, P3, Pz, P4, and POz.

In an example, with the possible exception of reference sites, the brain activity monitoring component of a device can comprise a wearable array of thirteen brain activity sensors. In an example, this array can be located substantially at the following placement sites: F3, Fz, F4, T3/T7, C3, C4, Cz, T4/T8, P3, P4, O1, and O2. In an example, with the possible exception of reference sites, a device can comprise a wearable array of ten brain activity sensors. In an example, this array can be located substantially at the following placement sites: FP1, FP2, F3, F4, T3/T7, T4/T8, P3, P4, O1, and O2.

In an example, a device can comprise a wearable array of eight brain activity sensors. In an example, this array can be placed substantially at the following placement sites: F3, F4, T3/T7, Cz, T4/T8, P3, Pz, and P4. In another example, this array can be located substantially at the following placement sites: F3, F4, C3, C4, Cz, Pz, O1, and O2. In another example, this array can be located substantially at the following placement sites: Fz, Cz, T5/P7, P3, Pz, P4, T6/P8, and Oz. In an example, a device can comprise a wearable array of seven brain activity sensors. In an example, a wearable array of brain activity sensors can be placed substantially at the following placement sites: FP1, FP2, Fz, C3, C4, Cz, and Pz. In another example, this array can be located substantially at the following placement sites: F3, F4, Cz, P3, P4, O1, and O2.

In an example, with the possible exception of reference sites, the brain activity monitoring component of a device can comprise a wearable array of six electrodes or other brain activity sensors. In an example, a wearable array of six brain activity sensors can be located substantially at the following placement sites: FP1, FP2, F7, F8, T3/T7, and T4/T8. In another example, this array can be placed substantially at: F3, F4, P3, P4, O1, and O2. In another example, a wearable array of brain activity sensors can be located substantially at the following placement sites: F3, F4, Cz, P2, O1, and O2. In another example, a wearable array of brain activity sensors can be located substantially at the following sites: F3, F8, T3/T7, T4/T8, T5/P7, and T6/P8. In another example, this array can be placed substantially at the following placement sites: FC3, T3/T7, C3, C4, Cz, and P3. In another example, this array can be located substantially at: T3/T7, T4/T8, T5/P7, T6/P8, O1, and O2. In an example, with the possible exception of reference sites, a device can comprise a wearable array of five brain activity sensors. In an example, this array can be located substantially at the following placement sites: AFz, F3, F4, CP5, and CP6. In another example, this array can be placed substantially at the following sites: F3, F4, Cz, P3, and P4. In another example, this array can be located substantially at the following placement sites: T3/T7, Cz, T4/T8, CP3, and CP4.

In an example, with the possible exception of reference sites, the brain activity monitoring component of a device can comprise a wearable array of four electrodes or other brain activity sensors. In an example, a wearable array of four brain activity sensors can be located substantially at: FP1, FP2, F7, and F8. In another example, this array can be placed substantially at the following placement sites: AF7, AF8, T3/T7, and T4/T8. In another example, this array can be located substantially at the following sites: F7, F3, F4, and F8. In another example, this array can be located substantially at the following placement sites: F7, F8, T3/T7, and T4/T8.

In an example, this array can be placed substantially at: F3, F4, P3, and P4. In another example, this array can be located substantially at the following placement sites: F3, Cz, P3, and O1. In another example, this array can be located substantially at the following sites: Fz, Cz, P3, and P4. In another example, this array can be placed substantially at the following placement sites: T3/T7, T4/T8, TP7, T5/P7, and T6/P8. In another example, this array can be located substantially at: T3/T7, T4/T8, PO7, and PO8. In another example, this array can be located substantially at the following placement sites: P3, P4, O1, and O2. In another example, this array can be placed substantially at the following sites: Cz, P3, Pz, and P4.

In an example, a wearable EEG monitor for measuring and/or modifying a person's food consumption can comprise: a plurality of electrodes or other brain activity sensors which are configured to be worn less than one inch from the surface of the person's head; and a control unit. In an example, a control unit can comprise: a mobile power source and/or power transducer (wherein a power transducer harvests power from human physiological activity and/or environmental energy sources), a data processor; and a data transmitter.

In an example, electrodes or other brain activity sensors can be located at the following EEG electrode sites: frontal polar sites Fp1 and Fp2; frontal sites F7, F3, Fz, F4 and F8; temporal sites T3 (T7 in the MCN), T4 (T8 in the MCN), T5 (P7 in the MCN) and T6 (P8 in the MCN); central sites C3, C4 and Cz; parietal sites P3, P4 and Pz; and occipital sites O1 and O2; and earlobe reference sites A1 and A2.

In an example, the configuration of electrodes or other brain activity sensors can be symmetric with respect to the left and right sides of the person's head. In an example, electrodes or other brain activity sensors can be located the following left-side and top sites: frontal polar site Fp1; frontal sites F7, F3 and Fz; temporal sites T3 and T5; central sites C3 and Cz; parietal sites P3 and Pz; and occipital site O1. Since symmetry is assumed, electrodes or other brain activity sensors are also assumed to be at the following right-side sites which are not shown here: frontal polar site Fp2; frontal sites F4 and F8; temporal sites T4 and T6; central site C4; parietal site P4; and occipital site O2. In an example, there can also be an optional earlobe reference site A1.

In an example, a wearable EEG monitor can comprise five arcuate bands (or straps). In an example, each of these five bands can arc around an approximately-hemispherical portion of the person's head. In an example, these five bands can converge at a pivoting location near the person's ear. In an example, a wearable EEG monitor can comprise three or four bands (or straps), each of which arcs around an approximately-hemispherical portion of the person's head. In various examples, these bands can be rigid, semi-rigid, flexible, or elastic. In an example, a bottom frontal band and a bottom posterior band together can comprise a slightly-curved, roughly-circular loop that goes around the person's head. In an example, this circular loop can be tilted at an acute angle with respect to the horizontal plane (when the person's head is upright), wherein this acute angle is in the range of 20 to 60 degrees.

In an example, the relative locations of a subset of bands can adjusted to customize the locations of electrodes or other brain activity sensors to the anatomy of a specific person. In an example, a subset of arcuate bands can be radially-adjusted by pivoting them around the point of band convergence. In an example, three upper bands can be radially-adjusted. In an example, a smaller or larger subset of bands can be radially-adjustable by pivoting. In an example, five bands can be radially-adjustable by pivoting.

In an example, radial-adjustment of bands can be done until the pattern of brain activity which is measured by the electrodes or other brain activity sensors best matches a standard or otherwise-expected pattern under selected conditions. In an example, such adjustment can be done in an iterative manner during a fitting period. In an example, radial-adjustment of one or more bands can be done by hand. In an example, radial-adjustment or one or more bands can be done automatically by one or more actuators. In an example, a control unit can include one or more actuators.

In an example, individual electrodes or other brain activity sensors can have fixed locations along the lengths of the bands. In an example, the location of one or more individual electrodes or other brain activity sensors can be moved along the lengths of bands, such as by movement along a track which spans a portion of the length of a band. In an example, this movement can be done by hand. In an example, this movement can be done automatically by one or more actuators. In example, this movement can help to customize the location of electrodes or other brain activity sensors to the anatomy of a specific person.

In an example, individual electrodes or other brain activity sensors can have fixed locations with respect to contact between the surface of a band and the surface of a person's head. In an example, individual electrodes or other brain activity sensors can be spring-loaded to maintain a desired level of contact or pressure with the surface of a person's head. In an example, the location, degree of contact, and/or level of pressure between one or more individual electrodes and the surface of a person's head can be adjusted. In an example, adjustment of the degree of contact and/or level of pressure between electrodes or other brain activity sensors and the surface of a person's head can help to customize an EEG monitor to the anatomy of a specific person. In an example, such contact or pressure adjustment can be done by hand. In an example, such contact or pressure adjustment can be done automatically by one or more actuators. In an example, such contact or pressure can be adjusted automatically by an actuator if proper contact or EM signal measurement is reduced or lost while the person is wearing the device.

In an example, all of the electrodes or other brain activity sensors which are physically present in an EEG monitor can be actively used to monitor a person's brain activity. In an alternative example, an EEG monitor can comprise a large array of electrodes or other brain activity sensors and only a subset of them may be used to monitor a specific person's brain activity. In an example, the selection of a subset of electrodes or brain activity sensors to be actively used can serve to customize electrode or brain activity sensor configuration to the anatomy of a specific person. In an example, selection of a subset of a larger array of electrodes for active use can serve to customize electrode placement for a specific person without having to physically move electrode locations.

In an example, an EEG monitor can comprise a large array of potential connection locations for electrodes or other brain activity sensors, but not all of these potential connection locations are used for electrodes or other brain activity sensors for a specific person. In an example, the selection of which connection locations to which electrodes or other brain activity sensors are actually connected can serve to customize the configuration of electrodes or brain activity sensor for a specific person. In an example, the selection of which connection locations are used for electrodes or brain activity sensors can customize an EEG monitor to the anatomy of a specific person. In an example, individual electrodes or brain activity sensors can be selectively inserted by hand into a subset of a large array of connection locations in order to customize the placement of electrodes or other brain activity sensors for use for a specific person. In an example, this can allow customization of electrode or sensor placement for a specific person without having the expense of electrodes or sensors which are not used.

In an example, a control unit can be located where bands converge. In an alternative example, a control unit can be located at a different location, such as along the length of one of the bands. In an example a control unit can be located at a position in front of a person's ear. In an example, a control unit can be incorporated into an eyewear frame which is physically-integrated into an EEG monitor as part of the same device or is wirelessly-integrated with an EEG monitor as part of an overall system for measuring and/or modifying a person's food consumption.

In an example, a wearable EEG monitor and/or control unit can further comprise one or more components selected from the group consisting of: a power source and/or power-transducing component; a wireless data transmission and data reception component; a data memory component; and a data processor. In an example, wearable EEG monitor and/or control unit can further comprise one or more components selected from the group consisting of: a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; one or more accelerometers; one or more cameras; and a GPS component.

In an example, a wearable EEG monitor and/or control unit can further include one or more sensors selected from the group consisting of: accelerometer, inclinometer, gyroscope, strain gauge, or other motion or position sensor; microphone or other sound sensor; thermometer or other temperature sensor; camera or other imaging sensor; optical sensor or optoelectronic sensor; blood pressure sensor; ECG/EKG sensor, heart rate monitor, and/or heart rate sensor; EMG sensor or other muscle activity sensor; GPS sensor, other location sensor, magnetometer, or compass; spectroscopy sensor or other spectral analysis sensor; electrochemical sensor; blood oximetry sensor; piezoelectric sensor; chewing sensor or swallowing sensor; respiration sensor; pressure sensor; galvanic skin response sensor; and taste or odor sensor.

In an example, there can be wires or other conductive connections between a control unit and electrodes or other brain activity sensors. In an alternative example, electrodes or other brain activity sensors can be in wireless communication with a control unit.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a method, device, or system can include a first (calibration) time period during which data concerning food consumption and data concerning brain activity are independently collected and significant associations are identified between patterns of food consumption and patterns of brain activity. Next comes a second (post-calibration) time period during which these associations are used in combination with subsequent brain activity data in order to measure and/or modify the person's food consumption. Collectively, this comprises a method, device, or system for measuring and/or modifying a person's food consumption using a wearable EEG monitor.

In an example, the sight of food can evoke a response in a person's brain and a change in the person's electromagnetic brain activity. In an example, this change in brain activity can be called a first-phase of brain activity response to food because it is caused by the sight (and/or smell) of food before the person eats the food. In an example, this change in electromagnetic brain activity can be measured and/or recorded by a plurality of electrodes or other brain activity sensors on an EEG monitor.

In an example, the taste, smell, and tactile sensations of eating food can evoke a second and/or continued response in the person's brain and a second and/or continued change in the person's electromagnetic brain activity. This change in the person's brain activity can be called a second-phase of brain activity response to food because it is triggered when the person actually starts eating food. This change in electromagnetic brain activity is measured and/or recorded by the plurality of electrodes or other brain activity sensors on the EEG monitor.

In an example, a person can manually enter information concerning food into a handheld electronic device. In an example, manual entry of data can be done using a touch screen. In an example, manual entry of information concerning food consumed can occur during a first (calibration) time period. In various examples, a person can manually enter data concerning the types and amounts of food which they eat into a smart phone, electronic tablet, laptop, smart watch, smart wrist band, electronically-functional eyewear, or other electronic device. In various examples, a person can enter food consumption data via a touchscreen, speech recognition interface, gesture recognition interface, EMG recognition interface, eye movement recognition interface, keypad, buttons, or knobs.

In an example, a handheld electronic device and a wearable EEG monitor can be in wireless communication with each other. In an example, a wearable EEG monitor and a handheld electronic device together can comprise a system for measuring and/or modifying a person's food consumption. Wireless communication between the EEG monitor and the electronic device enables data concerning food consumption (having been entered into the device) and data concerning brain activity (having been recorded by the monitor) to be chronologically linked and analyzed to identify associations between patterns of food consumption and patterns of electromagnetic brain activity.

In an example, the data processing that is required to analyze this data and identify these associations can occur in a control unit of a wearable EEG monitor, in a data processor within a handheld device, or in a remote computer with which either the EEG monitor or the handheld device is in wireless communication. In an example, data concerning food consumption and brain activity can be analyzed to create a food-brainwave database which links specific patterns of food consumption with specific patterns of brain activity.

In an example, manual entry of food consumption information can be facilitated by an application on the handheld electronic device which provides a menu of common food items, including their images and descriptions. In an example, food consumption data can be collected and/or supplemented by having the person manually take pictures of food before a meal and a picture of any unconsumed food that remains after the meal. In an example, food consumption data can be collected and/or supplemented by analysis of images which are automatically taken by electronically-functional eyewear or a wearable camera. In an example, the amount of food eaten can be estimated by the difference in food volume between a before-meal food image and an after-meal food image.

In an example, food consumption data can be collected and/or supplemented by the use of a smart food utensil, mobile food probe, or mobile food scanner. In an example, a smart food utensil, mobile food probe, or mobile food scanner can include a spectroscopic sensor for analyzing the chemical composition of food. In an example, food consumption data can be collected and/or supplemented by scanning a bar code or other unique content on food packaging or a restaurant menu.

During a first (calibration) time period, data concerning a person's food consumption can be collected by a means other than analysis of electromagnetic brain activity. Also, during the first (calibration) time period data concerning the person's electromagnetic brain activity can be collected using a wearable EEG monitor. Data concerning food consumption and brain activity can then be jointly analyzed to identify significant associations between food consumption patterns and brain activity patterns.

In various examples, a first-phase change and a second-phase change in a person's electromagnetic brain activity can be measured and analyzed separately, sequentially, or jointly in order to link patterns of food consumption to patterns of brain activity. In an example, two phases of food consumption (before eating and during eating) can be measured and analyzed. In an alternative example, only one phase of food consumption may be measured and analyzed. In an alternative example, three phases of food consumption (before eating, during eating, and after digestion) may be measured and analyzed. In an example, changes can be linked to food consumption in general, without identifying a specific type of food. In an example, changes can be linked to the consumption of specific types and amounts of food, ingredients, and/or nutrients, such as the specific types and amounts of ingredients in food.

Operation of this hardware can include a second (post-calibration) time period during which associations between food consumption and brain activity are used, in combination with monitored brain activity data, to measure and/or modify the person's food consumption. In an example, these associations can be embodied in a food-brainwave database. In an example, these associations can be embodied in a statistical model with estimated parameters.

In an example, associations and/or a food-brainwave database can be created de novo for person during a first (calibration) time period for a person. In an alternative example, associations and/or a food-brainwave database can be created for a general population and then adapted for use for a person. In an example, use of population-based associations and/or a population-based food-brainwave database can reduce or entirely eliminate the need for a first (calibration) time period for a person.

In an example, a person can eat food during a second (post calibration) time period. In an example, there may be no independent entry of food consumption information during this second (post calibration) time period. During this second (post calibration) time period, food consumption is estimated based on the person's electromagnetic brain activity. In an example, the person's consumption of food can trigger a change in the person's electromagnetic brain activity. This change in brain activity is measured by a wearable EEG monitor.

In an example, data concerning the person's brain activity, including a change in response to consumption of food, can be wirelessly transmitted from a control unit of a wearable EEG monitor to a handheld electronic device. In an example, data concerning the person's brain activity during an extended period of time can be measured and recorded. In an example, this extended period of time can be several hours or an entire day. In an example, this extended period of time can include multiple food consumption events, of which eating food is only one event. In an example, brain activity data can be wirelessly transmitted from a wearable EEG monitor to a handheld electronic device at regular time intervals during an extended period of time, after each food consumption event, or at the end of the extended period of time.

In an example, data concerning changes in a person's brain activity for an extended period of time can be used to estimate the person's cumulative food consumption during this period of time. In this manner, a log of the person's food consumption can be automatically created for this period of time. In an example, this automatic log of food consumption can be at the level of overall food consumption and/or total calories. In an example, this automatic log of food consumption can be at the level of specific types and amounts of foods, ingredients, and/or nutrients.

In an example, a system or device can convey feedback to a person in order to modify their food consumption. In an example, a person can receive visual feedback concerning their food consumption from a handheld electronic device. In an example, feedback can be a warning that the person has consumed an unhealthy type or amount of food. In an example, feedback can be a warning that the person has not consumed a selected healthy food. In an example, feedback can indicate that the person has eaten too many calories overall compared to their caloric expenditure during a selected period of time. In an example, feedback can indicate that the person has eaten an unhealthy ingredient and/or an ingredient to which they are allergic.

In an example, feedback can be a text message. In an example, feedback can comprise a negative expression on an animated face or avatar. In example, feedback can include positive suggestions for how the person can modify their food consumption in a healthier manner in order to achieve their health goals. In other examples, feedback to modify a person's food consumption can be conveyed to the person through a different visual, auditory, or tactile interface.

In an example, a person can see food in a bowl, but turn away from the food rather than eating it because of feedback which the person receives. In an example, feedback may indicate that the person has eaten too many fatty snacks today and should avoid eating more fatty snacks. In an example, the person may turn away from a bowl of fatty snacks after having received such feedback. In an example, a device and system for modifying a person's food consumption using a wearable EEG monitor can help to strengthen a person's willpower to avoid over-consumption of unhealthy types or amounts of food.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a smart phone, tablet, or other electronically-functional handheld device.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a smart phone, tablet, or other electronically-functional handheld device; and a database which includes associations between specific patterns of food consumption and specific patterns of electromagnetic brain activity, which translates specific types and amounts of food into specific types and amounts of nutrients, or which does both.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; and a visual interface for computer-to-human communication.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a visual interface for computer-to-human communication; and a database which includes associations between specific patterns of food consumption and specific patterns of electromagnetic brain activity, which translates specific types and amounts of food into specific types and amounts of nutrients, or which does both.

In an example, a wearable EEG monitor can be used to modify a person's food consumption. In an example, this can involve interactive feedback concerning the person's electromagnetic brain activity patterns. In an example, a person can modify their food consumption by self-modifying their electromagnetic brain activity patterns. In an example, hardware can be used to implement the biofeedback methods that were disclosed earlier.

In an example, a person can interact with a handheld electronic device which, in turn, is in wireless communication with a wearable EEG monitor. In an example, this can result in a self-modified change in the person's electromagnetic brain activity from a first pattern to a second pattern. In an example, the first pattern can be an electromagnetic brain activity pattern which is generally associated with hunger and the second pattern can be an electromagnetic brain activity pattern which is generally associated with satiety.

In an example, a person can receive interactive feedback concerning their electromagnetic brain activity pattern as measured by wearable EEG monitor, wirelessly transmitted to handheld electronic device, and visually displayed on the screen of electronic device. In an example, the visual display on the electronic device can change as the person's brain activity pattern changes. In an example, changes in the visual display can help the person to self-modify their brain activity pattern in a way which modifies their food consumption. In an example, when the visual display is changed to a selected configuration, this indicates that the person's brain activity has changed from a first pattern to a second pattern. In an example, when the person has modified the visual display to a selected configuration, then this indicates that the person has modified their brain activity pattern to one which is generally associated with satiety.

In an example, a person's self-modification of their electromagnetic brain activity can successfully modify their food consumption behavior. In an example, a person who has seen food in a bowl can turn away from the food rather than eat it. In an example, the person can be enabled to turn away from the food because they have self-modified their electromagnetic brain activity to a pattern which is generally associated with satiety. In an example, this is a case of mind over platter.

In an example, an interactive feedback by which the person self-modifies their brain activity can be visual feedback via a handheld electronic device. In another example, interactive feedback can be embodied in audio feedback—such as a series or tones or music whose parameters are changed by changes in measured brain activity. In another example, interactive feedback can be embodied in tactile feedback—such as a series of vibrations or pressure points applied to the person's skin by a wearable electronic device.

In an example, a system can comprise a wearable EEG monitor and a smart watch (or other electronically-functional wrist band) which are in wireless communication with each other. In an example, a wearable EEG monitor and a smart watch (or wrist band) together can comprise a wirelessly-linked system for measuring and/or modifying a person's food consumption.

In an example, a smart watch (or wrist band) can comprise a convenient user interface for a system. In an example, a smart watch (or wrist band) can comprise a human-to-computer interface for a system for entering information concerning food consumption during a first (calibration) period. In an example, a smart watch (or wrist band) can house key data processing and/or transmitting components for this system. In an example, a smart watch (or wrist band) can comprise a computer-to-human interface for providing feedback to the person from the system in order to modify the person's food consumption.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a smart watch and/or electronically-functional band which is configured to be worn on a person's wrist.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a smart watch and/or electronically-functional band which is configured to be worn on a person's wrist; and a database which includes associations between specific patterns of food consumption and specific patterns of electromagnetic brain activity, which translates specific types and amounts of food into specific types and amounts of nutrients, or which does both.

In an example, a system can comprise a wearable EEG monitor and electronically-functional eyewear which are in wireless communication with each other. In an example, a wearable EEG monitor and eyewear together can comprise a wirelessly-linked system for measuring and/or modifying a person's food consumption.

In an example, electronically-functional eyewear can comprise a convenient user interface for this system. In an example, electronically-functional eyewear can comprise a wearable camera whose images are analyzed to provide information concerning food consumption during a first (calibration) period. In an example, electronically-functional eyewear can house key data processing and/or transmitting components for this system. In an example, electronically-functional eyewear can comprise a computer-to-human interface for providing augmented reality visual feedback to modify the person's food consumption.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a pair of smart glasses and/or electronically-functional eyewear which is configured to be worn on a person's head.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a pair of smart glasses and/or electronically-functional eyewear which is configured to be worn on a person's head; and a database which includes associations between specific patterns of food consumption and specific patterns of electromagnetic brain activity, which translates specific types and amounts of food into specific types and amounts of nutrients, or which does both.

In an example, a system can comprise a wearable EEG monitor and a neck-worn device which are in wireless communication with each other. In an example, a wearable EEG monitor and a neck-worn device together can comprise a wirelessly-linked system for measuring and/or modifying a person's food consumption.

In an example, a neck-worn device can comprise an electronically-functional necklace and/or a wearable camera. In an example, a neck-worn device comprising a wearable camera can take pictures of food as a person eats. Analysis of such food images can provide food consumption tracking during a first (calibration) time period. In an example, a neck-worn device can further comprise a microphone. In an example, to help preserve visual privacy, a wearable camera may only be activated to take pictures when a microphone detects chewing and/or swallowing sounds which indicate that the person is probably eating. In an example, a neck-worn device can further comprise a convenient user interface for this system and/or house key data processing components for this system.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a smart necklace and/or electronically-functional device which is configured to worn on a person's neck.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a smart necklace and/or electronically-functional device which is configured to worn on a person's neck; and a database which includes associations between specific patterns of food consumption and specific patterns of electromagnetic brain activity, which translates specific types and amounts of food into specific types and amounts of nutrients, or which does both.

In an example, a system can comprise a wearable EEG monitor and an intraoral food sensor. These two components of the system can be in wireless communication with each other. Together, a wearable EEG monitor and an intraoral food sensor can comprise a wirelessly-linked system for measuring and/or modifying a person's food consumption. In an example, an intraoral food sensor can be attached to, or implanted within, the palatal vault of the person's mouth. In an example, intraoral food sensor can be attached to the person's teeth, gums, or jaw. In an example, intraoral food sensor can be attached to, or implanted within, the person's tongue. In an example, intraoral food sensor can be in fluid communication with saliva and other intraoral fluids and materials.

In an example, an intraoral food sensor can analyze micro-samples of intraoral fluids in order to determine their chemical composition. In an example, for better operational efficiency, an intraoral food sensor may only analyze micro-samples of intraoral fluids when other sensors (such as chewing sensors or swallow sensors) suggest that the person is eating something.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a device which is configured to be in fluid communication with a person's mouth or other portion of the person's gastrointestinal tract.

In an example, a system for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a device which is configured to be in fluid communication with a person's mouth or other portion of the person's gastrointestinal tract; and a database which includes associations between specific patterns of food consumption and specific patterns of electromagnetic brain activity, which translates specific types and amounts of food into specific types and amounts of nutrients, or which does both.

In an example, a wearable EEG monitor can be used to measure and/or modify a person's food consumption. In an example, such a device can be embodied as a hybrid wearable EEG monitor with an integrated eyewear frame. In an example, a wearable EEG monitor can serve as both a brain activity monitor and electronically-functional eyewear. In an example, a wearable EEG monitor can comprise: a plurality of electrodes or other brain activity sensors; a control unit; and a wearable camera. In an example, the control unit can further comprise a power source, a data processor, and a wireless data transmitter.

In an example, an anterior portion of a wearable EEG monitor can serve as an eyewear frame and the posterior portion of wearable EEG monitor can loop completely around the back of a person's head. In an example, the front of a device can rest on the bridge of a person's nose and the sides of the device can rest on the person's ears. In an example, the anterior and posterior portions of a device can form a continuous arcing band which encircles a person's head at a relatively-constant level which is just above the person's nose and ears.

In an example, an eyewear portion of a device can have lenses, but no display screen. In another example, the eyewear portion of this device can have a display screen, but no lenses. In another example, the eyewear portion of this device may have both a display screen and lenses. In an example, the eyewear portion of this device can serve as an augmented reality interface.

In an example, a device for measuring and/or modifying a person's food consumption can comprise: eyewear which is configured to be worn on a person's head; one or more electrodes or other brain activity sensors which are configured by the eyewear to be less than one inch from the surface of the person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: eyewear which is configured to be worn on a person's head; one or more electrodes or other brain activity sensors which are configured by the eyewear to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a wearable EEG monitor for measuring and/or modifying a person's food consumption can include one or more wearable cameras. In an example, a wearable camera can be positioned on a side of a person's head near the point where multiple bands of the monitor converge. In an example, two wearable cameras positioned on the left and right sides of the person's head can provide stereoscopic images of food during a first (calibration) time period. These images can be analyzed to estimate food consumption during the calibration period. In an example, visual pattern recognition can be used to determine food types. In an example, 3D modeling of stereoscopic images can be used to estimate food amounts.

In an example, a wearable camera can take video pictures continually. In an example, in order to help maintain visual privacy, a wearable camera may only be activated to take video pictures when data from other sources (such as motion sensors or sound sensors) suggests that the person is probably eating. In an example, in order to help maintain visual privacy, a wearable camera may have a short focal length.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a wearable camera which is configured to record images of food consumption by a person.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a wearable camera which is configured to record images of food consumption by a person; and a database which includes associations between specific patterns of food consumption and specific patterns of electromagnetic brain activity, which translates specific types and amounts of food into specific types and amounts of nutrients, or which does both.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a wearable camera which is configured to record images of food consumption by a person, wherein this camera is activated to take pictures when the person's electromagnetic brain activity pattern indicates probable food consumption.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a wearable camera which is configured to record images of food consumption by a person, wherein this camera is activated to take pictures when the person's electromagnetic brain activity pattern indicates probable food consumption; and a database which includes associations between specific patterns of food consumption and specific patterns of electromagnetic brain activity, which translates specific types and amounts of food into specific types and amounts of nutrients, or which does both.

In an example, a wearable EEG monitor can integrated eyewear and two upper arcuate members which loop over the top of the person's head. The addition of these two upper arcuate members can increase the range of electrodes or other brain activity members covering the person's head. The addition of these two upper arcuate members provides electromagnetic recording coverage of the person's parietal lobe and upper occipital lobe. This can provide improved measurement of brain activity related to hunger and auditory sensation. In an example, a control unit can be located in front of a person's ears. In an example, a control unit can further comprise a power source, a data processor, a data transmitter, and a user interface.

In an example, bottom portions of two upper arcuate members can converge at locations just above a person's ears. In an example, the upper portions of the two upper arcuate members can diverge, at an angle in the range of 20 to 80 degrees, as they leave a convergence location and loop around the top of the person's head. In various examples, these two upper arcuate members can be rigid, semi-rigid, flexible, or elastic. In various examples, these two upper arcuate members can be made of metal, plastic, or fabric.

In an example, a wearable EEG monitor for measuring and/or modifying a person's food consumption can also serve as electronically-functional eyewear. In an example, the anterior portion of a wearable EEG monitor can comprise an eyewear frame. In an example, the posterior portion of the wearable EEG monitor can comprise two rear arcuate members which diverge from locations near the person's ears to loop around the back of the person's head. In an example, these two rear arcuate members can diverge at an angle in the range of 20 to 80 degrees as they loop around the back of the person's head.

In an example, a wearable EEG monitor for measuring and/or modifying a person's food consumption can comprise: a plurality of electrodes or other brain activity sensors; a control unit; and a wearable camera. In an example, the control unit can further comprise a power source, data processor, a data transmitter, and a user interface. In various examples, two rear arcuate members can be rigid, semi-rigid, flexible, or elastic. In various examples, these two rear arcuate members can be made of metal, plastic, or fabric.

In an example, a wearable EEG monitor for measuring and/or modifying a person's food consumption can comprise two rear arcuate members, wherein at least one of the two rear arcuate members can be radially-pivoted around their convergence point. This pivoting action changes the angle at which the two rear arcuate members diverge as they loop around the back of the person's head. This pivoting action enables customization and/or adjustment of the locations of electrodes or other brain activity sensors which span the person's occipital lobe and/or parietal lobe. In an example, this device can also comprise a control unit which further comprises a power source, a data processor, and a data transmitter.

In an example, a wearable EEG monitor can comprise a plurality of electrodes or other brain activity sensors and a wearable camera. In an example, a device can also comprise a control unit which further comprises a power source, a data processor, and a data transmitter. In an example, the anterior portion of the wearable EEG monitor can be an eyewear frame. In an example, the posterior portion of the wearable EEG monitor can: start near the bottom of the left ear, curve up around the rear of the left ear, loop over the top of the person's head, curve down around the rear of the right ear, and then end near the bottom of the right ear. In an example, the anterior portion of the device can rest on the bridge of the person's nose and the posterior portions of the device can rest on the tops of the person's ears.

In an example, a wearable EEG monitor can comprise a plurality of electrodes or other brain activity sensors and a wearable camera. In an example, this device can also comprise a control unit which further comprises a power source, a data processor, and a data transmitter. In an example, the anterior portion of the wearable EEG monitor can be an eyewear frame. In an example, the device can: span backward from the bridge of the person's nose to a position atop the person's left ear; then curve up and over the top of the person's head; then curve down to a position atop the person's right ear; and then span forward to the bridge of the person's nose. In an example, the anterior portion of this device can rest on the bridge of the person's nose and the side portions of this device can rest on the tops of the person's ears.

In an example, a person can first see (and smell) food before they start to eat it. Then, the person eats food which triggers a change in their electromagnetic brain activity which is recorded by electrodes on a wearable EEG monitor. In an example, this can trigger a camera to take pictures of food. In an example, the system can use previously-identified associations between food consumption and brain activity to identify the types and amounts of food consumed during a period of time. In an example, the system can automatically create a log of food consumption during this period of time.

In an example, a person can receive feedback from a device or system via a handheld electronic device in order to modify their food consumption. In an example, a wearable EEG monitor and a handheld electronic device can be in wireless communication. In an example, an automatically-created food log based on brain activity patterns can indicate that a person has eaten an unhealthy type and/or amount of food during a period of time. In an example, feedback based on this log may suggest that the person should modify their food consumption to improve their nutrition, manage their energy balance, and/or meet health goals.

In an example, a person can directly modify their food consumption based on feedback. In another example, a person can indirectly modify their food consumption via self-modification of brain activity. In this latter example the person uses an interactive biofeedback interface to self-modify their brain activity pattern which, in turn, modifies their food consumption. In various examples, an interactive biofeedback interface can be visual, auditory, and/or tactile. In an example, a wearable EEG monitor and a handheld electronic device can share interactive wireless communication.

In an example, person can use interactive biofeedback interface to self-modify their brain activity pattern from a first pattern which is generally associated with hunger to a second pattern which is generally associated with satiety. In an example, when the person is able to self-modify their brain activity to a pattern associated with satiety, they can better avoid eating an unhealthy amount or type of food. In an example, a person can initiate use of biofeedback interface based on feedback. In another example, a person can initiate use of this biofeedback interface on their own, without prompting by feedback from a system.

In an example, a person can turn away from a bowl of food instead of eating it. In an example, this modification of food consumption can be a direct result of feedback. In an example, this modification of food consumption can be an indirect result of self-modification of brain activity patterns with the help of an interactive biofeedback interface. In an example, this modification of food consumption can be the combined result of direct feedback and interactive biofeedback.

In an example, a wearable EEG monitor for measuring and/or modifying a person's food consumption can integrate both eyewear and headphone components. In an example, a wearable EEG monitor can comprise a plurality of electrodes or other brain activity sensors and wearable camera. In an example, this monitor can further comprise a control unit which, in turn, can comprise a power source, a data processor, and a data transmitter. In an example, the anterior portion of wearable EEG monitor can comprise eyewear frames. In an example, the posterior portion of wearable EEG monitor can comprise a set of headphones which cover a person's ears and loop over the top of the person's head. In an example, an eyewear frame and headphones can be integrated into a single device.

In an example, a wearable EEG monitor for measuring and/or modifying a person's food consumption can be shaped like headphones which cover a person's ears and loop over the top of their head. A wearable EEG monitor can comprise a plurality of electrodes or other brain activity sensors and one or more wearable cameras. In an example, this monitor can further comprise a control unit which, in turn, can comprise a power source, a data processor, and a data transmitter.

In an example, a wearable EEG monitor can be used to measure and/or modify a person's food consumption. In an example, one or more wearable cameras can take pictures of food before it is eaten. Then, the one or more wearable cameras can take pictures of food while it is being eaten. Electrodes or other brain activity sensors can record a change in electromagnetic brain activity which is caused by the consumption of the food.

In an example, a wearable EEG monitor can be in wireless communication with a handheld electronic device and convey feedback concerning food consumption to a person. In an example, a wearable EEG monitor can be in wireless communication with a handheld electronic device and help the person to self-modify their brain activity using an interactive biofeedback interface. In an example, biofeedback can help the person to self-modify their brain activity from a first pattern that is associated with hunger to a second pattern that is associated with satiety. In an example, a person may turn away from food due to feedback, self-modification of brain activity to a satiety pattern, or both.

In an example, a device for measuring and/or modifying a person's food consumption can comprise: a set of headphones or hair band which is configured to be worn over the top of a person's head; one or more electrodes or other brain activity sensors which are configured by the set of headphones or hair band to be less than one inch from the surface of the person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: a set of headphones or hair band which is configured to be worn over the top of a person's head; one or more electrodes or other brain activity sensors which are configured by the set of headphones or hair band to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a headphone-style wearable EEG monitor can include a microphone. In an example, the wearable EEG monitor can detect eating by detecting chewing and/or swallowing sounds. In an example, a headphone-style wearable EEG monitor can have an interactive biofeedback interface which is auditory in nature. In an example, changes in sound tones and/or musical parameters can help a person to self-modify their brain activity pattern from a first pattern to a second pattern. This change in brain activity pattern, in turn, modifies their food consumption.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a microphone.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a microphone; and a database which includes associations between specific patterns of food consumption and specific patterns of electromagnetic brain activity, which translates specific types and amounts of food into specific types and amounts of nutrients, or which does both.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; and an auditory interface for computer-to-human communication.

In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; an auditory interface for computer-to-human communication; and a database which includes associations between specific patterns of food consumption and specific patterns of electromagnetic brain activity, which translates specific types and amounts of food into specific types and amounts of nutrients, or which does both.

In an example, a wearable EEG monitor can comprise: a plurality of electrodes or other brain activity sensors; and one or more wearable cameras. In an example, the wearable EEG monitor can further comprise a control unit. In an example, a control unit can further comprise a power source, a data processor, and a data transmitter. In an example, a wearable EEG monitor can encircle a person's head in a sinusoidal manner and rest on the tops of the person's ears. In an example, a wearable EEG monitor can comprise two wearable cameras, on the left and right sides of the person's head, in order to take stereoscopic pictures of food. Stereoscopic pictures of food can be useful for 3D modeling to better estimate food volume and the amount of food consumed by the person. In an example, consumption of food can trigger a change in a person's electromagnetic brain activity. This change can be measured by a plurality of electrodes or other brain activity sensors.

In an example, a device for measuring and/or modifying a person's food consumption can comprise: a headband which is configured to be worn around a person's head; one or more electrodes or other brain activity sensors which are configured by the headband to be less than one inch from the surface of the person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: a headband which is configured to be worn around a person's head; one or more electrodes or other brain activity sensors which are configured by the headband to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a device for measuring and/or modifying a person's food consumption can comprise: a headband which is configured to be worn around a person's head; one or more electrodes or other brain activity sensors which are configured by the headband to be less than one inch from the surface of the person's head; a mobile power source and/or power transducer, wherein a power transducer harvests power from human physiological activity and/or environmental energy sources; a data processor; a data transmitter; and a plurality of electromagnetic signal transmitters which are configured to be less than one inch from the surface of a person's head, wherein these electromagnetic signal transmitters collectively modify an electromagnetic field in order to reproduce a pattern of brain activity which is associated with satiety and/or consumption of a specific type of food, ingredient, or nutrient.

In an example, a device for measuring and/or modifying a person's food consumption can comprise: a headband which is configured to be worn around a person's head; one or more electrodes or other brain activity sensors which are configured by the headband to be less than one inch from the surface of the person's head; a mobile power source and/or power transducer, wherein a power transducer harvests power from human physiological activity and/or environmental energy sources; a data processor; a data transmitter; a plurality of electromagnetic signal transmitters which are configured to be less than one inch from the surface of a person's head, wherein these electromagnetic signal transmitters collectively modify an electromagnetic field in order to reproduce a pattern of brain activity which is associated with satiety and/or consumption of a specific type of food, ingredient, or nutrient; and a database which includes associations between specific patterns of food consumption and specific patterns of electromagnetic brain activity, which translates specific types and amounts of food into specific types and amounts of nutrients, or which does both.

In an example, a wearable EEG monitor can be used to measure and/or modify a person's food consumption. In an example, a wearable EEG monitor can comprise: a plurality of electrodes or other brain activity sensors; and one or more wearable cameras. In an example, wearable EEG monitor can further comprise a control unit. In an example, a control unit can further comprise a power source, a data processor, and a data transmitter. In an example, a wearable EEG monitor can comprise an upper band which loops around the upper back of a person's head at a level above the person's ears and a lower band which loops around the lower back of the person's head at a level equal to, or lower than, the person's ears, wherein the ends of these two bands converge at locations above the person's ears and rest on the tops of the person's ears. In an example, a wearable EEG monitor can comprise two wearable cameras, on the left and right sides of the person's head, in order to take stereoscopic pictures of food.

In an example, a wearable EEG monitor can double as eyewear and be used to measure and/or modify a person's food consumption. In an example, a wearable EEG monitor can comprise a plurality of electrodes or other brain activity sensors and two wearable cameras. In an example, a device can be left-right symmetric. In an example, a wearable EEG monitor can further comprise a control unit. In an example, this control unit can comprise a power source, data processor, and data transmitter.

In an example, an anterior portion of a wearable EEG monitor can comprise an eyewear frame. In an example, this eyewear frame can include lenses. In an example, this eyewear frame can include a display surface instead of lenses. In an example, lenses can function as a display surface. In an example, this eyewear frame can be rigid, semi-rigid, or flexible.

In an example, a posterior portion of a wearable EEG monitor can comprise an arcuate member which loops around the lower-rear portion of the back of a person's head at a level which is equal to, or lower than, the person's ears. In an example, the sides of this device rest on top of the person's ears. In an example, the posterior arcuate portion of the device can have the same degree of rigidity, flexibility, and/or elasticity as the anterior eyewear frame portion of this device. In another example, a posterior arcuate portion of this device can have a higher degree of flexibility and/or elasticity than an anterior eyewear frame portion of this device. In an example, the anterior eyewear frame portion of this device can be made of metal and/or plastic and the posterior arcuate portion of this device can be made of fabric.

In an example, two wearable cameras on a device can take stereoscopic pictures of food when a person is looking at food and when the person is eating food. In an example, having images of food both before and during consumption can enable more accurate identification of food type and more accurate measure of food quantity consumed. Also, stereoscopic imaging of food can enable 3D and volumetric modeling to better estimate the quantity of food consumed.

In an example, a change in electromagnetic brain activity can be triggered when a person eats food. This change in electromagnetic brain activity can be measured by a wearable EEG monitor. This change in brain activity based on food consumption can then linked to previously-identified patterns of food consumption and used to estimate the type and quantity of food consumed.

In an example, a wearable EEG monitor can comprise a crown-like sinusoidal headband (which encircles a person's head) with a plurality of electrodes or other brain activity sensors. In an example, eating food can trigger a change in a person's electromagnetic brain activity, wherein this change is measured by the plurality of electrodes or other brain activity sensors.

In an example, a wearable EEG monitor can comprise: a saddle-shaped section which loops over the top of the person's head (figuratively appearing as if a central oval or elliptical loop has been melted on the top of the person's head and droops down the sides of the person's head); and two arcs which extend from the bottom portions of the saddle-shaped section to curve down around the rear portions of the person's ears. This monitor can have a plurality of electrodes or other brain activity sensors and one or more wearable cameras. In an example, eating food can trigger a change in a person's electromagnetic brain activity, wherein this change is measured by the plurality of electrodes or other brain activity sensors.

In an example, a wearable brain activity monitor can comprise a head-worn sensor-positioning member which is configured to position a plurality of electrodes or other brain activity sensors at selected locations on a wearer's head. In an example, a sensor-positioning member can be substantially symmetric with respect to the right and left sides of the wearer's head.

In an example, a sensor-positioning member can be configured to loop over the top portion of a wearer's head in a manner similar to the rim of a (skull) cap in which the right and left sides have been elongated. In an example, the right and left sides of the device can come down to locations just above the wearer's ears. In various examples, a sensor-holding member can be shaped like a two-dimensional circle, oval, ellipse, oblong shape, egg shape, or conic section which has been curved in three-dimensional space in order to conform to the top portion of the wearer's head.

In an example, a portion of sensor-positioning member that is anterior to the wearer's ears can span an upper portion of the wearer's temporal lobe, a lower portion of their central sulcus, and a posterior portion of their cerebral cortex. In an example, electrodes or other brain activity sensors on sensor-positioning member can collect data on brain activity concerning short term memory, emotion, smell, hunger, and/or taste. This brain activity data can be associated with selected quantities and types of food consumption and/or can be used to identify quantities and types of food consumed.

In an example, a portion of a sensor-positioning member that is posterior to a wearer's ears can span an upper portion of the wearer's temporal lobe and a posterior portion of their occipital lobe. In an example, electrodes or other brain activity sensors on the sensor-positioning member can collect data on brain activity concerning the sight, image recognition, and/or speech. This brain activity data can be associated with selected quantities and types of food consumption and/or can be used to identify quantities and types of food consumed.

In an example, a wearable brain activity monitor can comprise an array of eighteen electrodes or other brain activity sensors which are located substantially at the following set of placement sites—FC1, FC2, FC3, FC4, FC5, FC6, FCz, P1, P2, P3, P4, P5, P6, Pz, T7, T8, TP7 and TP8—or which comprise a subset of six or more sites from this set of placement sites.

In an example, a control unit can further comprise: a data processing component and a power source (or transducer). In an example, a control unit can further comprise: a data processing component; a power source (or transducer); and a data transmitting (and receiving) component. In an example, a control unit can be in wireless communication with an external (or remote) device and/or with another component of an overall system for monitoring brain activity. In an example, a control unit can further comprise: a data processing component; a power source (or transducer); a data transmitting (and receiving) component; and a user interface. In an example, a control unit can be physically connected to the array of electrodes (or other brain activity sensors) by wires or other electromagnetically-conductive pathways. In an example, a control unit can be in wireless electromagnetic communication with the array of electrodes (or other brain activity sensors).

In an example, a wearable brain activity monitor can comprise a head-worn sensor-positioning member which is configured to position a plurality of electrodes or other brain activity sensors at selected locations on a wearer's head. In an example, a sensor-positioning member can be substantially symmetric with respect to the right and left sides of the wearer's head. In an example, a control unit need not be replicated on both sides.

In an example, a sensor-positioning member can have two (substantially-parallel) arcs which loop over the top portion of a wearer's head. In an example, these two loops can be slightly concave with the concavity facing in an anterior direction. In another example, these two loops can be concave with the concavity facing in a posterior direction. In an alternative example, these two loops can be relatively straight. In an example, the ends of these two loops can connect to each other below the wearer's ears, on the right and left sides of the wearer's head, respectively.

In various examples, a sensor-holding member can be shaped like a two-dimensional oblong shape which has been curved in three-dimensional space in order to conform to the top portion of the wearer's head. In an example, a sensor-holding member can be shaped like a two-dimensional conic section which has been curved in three-dimensional space to conform to the top portion of the wearer's head.

In an example, an anterior loop of sensor-positioning member can span a laterally-central portion of a wearer's temporal lobe, a portion of their central sulcus, and a posterior portion of their cerebral cortex. In an example, electrodes or other brain activity sensors on a sensor-positioning member can collect data on brain activity concerning short term memory, emotion, smell, taste, and/or hunger. This brain activity data can be associated with selected quantities and types of food consumption and/or can be used to identify quantities and types of food consumed.

In an example, a posterior loop of a sensor-positioning member can span a portion of a wearer's cerebellum, a posterior portion of their temporal lobe, a laterally-central portion of their occipital lobe, and the upper tip of their parietal lobe. In an example, electrodes or other brain activity sensors on a sensor-positioning member can collect data on brain activity concerning hearing, sight, image recognition, speech, object weight, object texture, and/or object temperature. This brain activity data can be associated with selected quantities and types of food consumption and/or can be used to identify quantities and types of food consumed.

In an example, a wearable brain activity monitor can comprise an array of eighteen electrodes or other brain activity sensors which are located substantially at the following set of placement sites—CP1, CP2, CP3, CP4, CP5, CP6, CPz, FC1, FC2, FC3, FC4, FC5, FC6, FCz, P7, P8, T7 and T8—or which comprise a subset of ten or more sites from this set of placement sites.

In an example, a wearable brain activity monitor can comprise a head-worn sensor-positioning member which is configured to position a plurality of electrodes or other brain activity sensors at selected locations on a wearer's head. In an example, the sensor-positioning member can be substantially symmetric with respect to the left side and the right side of the wearer's head.

In an example, a sensor-positioning member can encircle an upper portion of a wearer's head in a tilted and sinusoidal manner. In various examples, a sensor-positioning member can encircle an upper portion of a wearer's head at an angle with respect to a horizontal plane when the wearer is standing upright which is within the range of 30 to 60 degrees. In an example, this angle is approximately 45 degrees. In various examples, a sensor-positioning member can have between 2 and 8 full-phase sinusoidal oscillations as it encircles the wearer's head. In an example, this member has four sinusoidal oscillations.

In an example, a portion of a sensor-positioning member which is anterior to the wearer's ears can span an upper portion of the wearer's temporal lobe, a central portion of their central sulcus, and a posterior portion of their cerebral cortex. In an example, electrodes or other brain activity sensors on a sensor-positioning member can collect data on brain activity concerning short term memory, emotion, smell, taste, and/or hunger. This brain activity data can be associated with selected quantities and types of food consumption and/or can be used to identify quantities and types of food consumed.

In an example, a portion of the sensor-positioning member which is anterior to the wearer's ears can span a lower portion of the wearer's occipital lobe, a posterior portion of their temporal lobe, and a laterally-central portion of their cerebellum. In an example, electrodes or other brain activity sensors on a sensor-positioning member can collect data on brain activity concerning speech, sight, image recognition, and/or hearing. This brain activity data can be associated with selected quantities and types of food consumption and/or can be used to identify quantities and types of food consumed.

In an example, a wearable brain activity monitor can comprise an array of seventeen electrodes or other brain activity sensors which are located substantially at the following set of placement sites—CP5, CP6, FC1, FC2, FC3, FC4, FC5, FC6, FCz, FT7, FT8, P5, P6, P7, P8, T7, T8—or which comprise a subset of six or more sites from this set of placement sites.

In an example, a wearable brain activity monitor can comprise two (wirelessly) linked head-worn sensor-positioning members which are configured to position a plurality of electrodes or other brain activity sensors at selected locations on the wearer's head. In an example, a first sensor-positioning member can be located above a second sensor-positioning member. In an example, these two sensor-positioning members can be substantially symmetric with respect to the right and left sides of a wearer's head.

In an example, sensor-positioning members can encircle an upper portion of a wearer's head in a tilted and sinusoidal manner. In an example, each of these sensor-positioning members can encircle an upper portion of a wearer's head at an angle with respect to the horizontal plane (when the wearer is standing upright) in the range of 30-60 degrees. In an example, each of these sensor-positioning members can have a number of sinusoidal oscillations as it encircles the wearer's head in the range of 3-7 oscillations.

In an example, a portion of a sensor-positioning member which is anterior to a wearer's ears can span an upper portion of the wearer's temporal lobe, a central portion of their central sulcus, and an upper portion of their cerebral cortex. In an example, a portion of a sensor-positioning member which is posterior to the wearer's ears can span an upper portion of the wearer's temporal lobe and a portion of their cerebellum.

In an example, a portion of a sensor-positioning member which is anterior to a wearer's ears can span a central portion of the wearer's occipital lobe and a portion of their parietal lobe. In an example, a portion of a sensor-positioning member which is posterior to the wearer's ears can span a lower portion of the wearer's occipital lobe and a posterior portion of the wearer's cerebellum.

In an example, electrodes or other brain activity sensors on sensor-positioning members can collect data on brain activity concerning short term memory, smell, taste, emotion, hunger, speech, skin sensation, sight, image recognition, and/or hearing. This brain activity data can be associated with selected quantities and types of food consumption and/or can be used to identify quantities and types of food consumed.

In an example, a wearable brain activity monitor can comprise an array of 26 electrodes or other brain activity sensors which are located substantially at the following set of placement sites—C1, C2, C3, C4, C5, C6, CP5, CP6, Cz, FC1, FC2, FC3, FC4, FC5, FC6, FCz, FT7, FT8, P5, P6, PO7, PO8, T7, T8, TP7 and TP8—or which comprise a subset of eight or more sites from this set of placement sites.

In an example, a wearable brain activity monitor can comprise a head-worn sensor-positioning member which is configured to position a plurality of electrodes or other brain activity sensors at selected locations on a wearer's head. In an example, a sensor-positioning member can be substantially symmetric with respect to the right and left sides of the wearer's head.

In an example, a sensor-positioning member can comprise: (a) two arcuate elements which loop in a substantially-parallel manner over the top of the wearer's head; (b) an arcuate element which loops around the back of the wearer's head; and (c) two downward-protruding arcuate elements, one on the left side and one on the right side (not shown but assumed in symmetry) of the wearer's head which terminate in the areas behind the wearer's left and right ears, respectively.

In an example, an anterior element of the two arcuate elements which loops over a wearer's head can span an upper portion of the wearer's temporal lobe, a central portion of their parietal lobe, and an upper-posterior tip of their cerebral cortex. In an example, the posterior element of the two arcuate elements which loops over the wearer's head can span a laterally-central portion of the wearer's occipital lobe and the upper tip of their parietal lobe. In an example, an element which loops around the back of the wearer's head can span a posterior portion of the wearer's temporal lobe and a posterior portion of their cerebellum. In an example, each of the downward protruding elements can span a posterior portion of the wearer's temporal lobe and a central portion of their cerebellum.

In an example, electrodes or other brain activity sensors collect data on brain activity concerning: short term memory, smell, taste, emotion, hunger, skin sensation, speech, hearing, object weight, object texture, object temperature, and/or sight, image recognition. This brain activity data can be associated with selected quantities and types of food consumption and/or can be used to identify quantities and types of food consumed. In an example, a wearable brain activity monitor can comprise an array of 25 electrodes or other brain activity sensors which are located substantially at the following set of placement sites—C1, C2, C3, C4, C5, C6, CP1, CP2, CP3, CP4, CP5, CP6, CPz, Cz, O1, O2, Oz, P7, P8, PO7, PO8, T7, T8, TP7 and TP8—or which comprise a subset of ten or more sites from this set of placement sites.

In an example, a sensor-positioning member can comprise: (a) three arcuate elements which loop over the top of the wearer's head; and (b) two downward protruding elements, one on the left side and one on the right side (not shown but assumed in symmetry) of the wearer's head, which terminate in the areas behind the wearer's left and right ears, respectively. In an example, a left-side view of the shape of sensor-positioning member can look like the Greek letter Psi.

In an example, an anterior one of the three elements which loops over the wearer's head can span an upper portion of a wearer's temporal lobe, a central portion of their parietal lobe, and a posterior-upper tip of their cerebral cortex. In an example, a central one of the three elements which loops over the wearer's head can span a laterally-central portion of the wearer's occipital lobe, including the somatosensory area. In an example, the posterior one of the three elements which loops over the wearer's head can span a posterior portion of the wearer's occipital lobe, including the visual processing area. In an example, each of the downward protruding elements can span a posterior portion of the wearer's temporal lobe and a portion of their cerebellum.

In an example, electrodes or other brain activity sensors collect data on brain activity concerning: short term memory, smell, taste, emotion, hunger, skin sensation, speech, hearing, object weight, object texture, object temperature, and/or sight, image recognition. This brain activity data can be associated with selected quantities and types of food consumption and/or can be used to identify quantities and types of food consumed. In an example, a wearable brain activity monitor can comprise an array of 27 electrodes or other brain activity sensors which are located substantially at the following set of placement sites—C1, C2, C3, C4, C5, C6, CP1, CP2, CP3, CP4, CP5, CP6, CPz, Cz, P1, P2, P3, P4, P5, P6, P7, P8, Pz, T7, T8, TP7 and TP8—or which comprise a subset of twelve or more sites from this set of placement sites.

In an example, a sensor-positioning member can comprise: (a) a ring element which encircles the top of the wearer's head in a manner like the rim of a (skull) cap; and (b) an arc element which loops over the top of the wearer's head in a manner like the upper portion of a pair of headphones. In an example, these two elements can be joined on the left side and right side at locations just over the wearer's left ear and right ear, respectively.

In an example, a portion of a ring element which is anterior to a wearer's ear can span an upper portion of the wearer's temporal lobe, a lower portion of their parietal lobe, a lower portion of their central sulcus, and a laterally-central portion of their cerebral cortex. In an example, electrodes or other brain activity sensors can collect data on brain activity concerning short term memory, smell, taste, hunger, speech, and/or eye movement.

In an example, the portion of the ring element which is posterior to the wearer's ear spans an upper portion of the wearer's temporal lobe and an upper-posterior portion of their cerebellum. In an example, electrodes or other brain activity sensors collect data on brain activity concerning short term memory and/or hearing. In an example, the arc element spans a laterally-central portion of the occipital lobe, including the somatosensory area. In an example, electrodes or other brain activity sensors collect data on brain activity concerning language, hearing, object weight, object texture, object temperature, sight, and/or image recognition.

Brain activity data from the above electrodes or other brain activity sensors can be associated with selected quantities and types of food consumption and/or can be used to identify quantities and types of food consumed. In an example, a wearable brain activity monitor can comprise an array of 23 electrodes or other brain activity sensors which are located substantially at the following set of placement sites—CP1, CP2, CP3, CP4, CP5, CP6, CPz, FC1, FC2, FC3, FC4, FC5, FC6, FCz, O1, O2, Oz, P7, P8, PO7, PO8, TP7 and TP8—or which comprise a subset of eight or more sites from this set of placement sites.

In an example, a wearable brain activity monitor can comprise a head-worn sensor-positioning member which is configured to position a plurality of electrodes or other brain activity sensors at selected locations on a wearer's head. In an example, a sensor-positioning member can comprise an arcuate loop which encircles the upper-posterior portion of the wearer's head. In an example, this sensor-positioning member can comprise: an anterior portion that curves over the top of the wearer's head in a manner similar to the upper portion of headphones; a laterally-central portion that partially reflects the curves of the upper and posterior perimeters of the wearer's ears; and a posterior portion that loops around the rear of the wearer's head at substantially the same height as the wearer's ears.

In an example, an anterior portion of this loop can span an upper portion of the wearer's temporal lobe, a lower portion of their parietal lobe, a portion of their central sulcus, and a posterior portion of their cerebral cortex. In an example, electrodes or other brain activity sensors can collect data on brain activity concerning short term memory, smell, taste, hunger, speech, and/or eye movement. In an example, a posterior portion of this loop can span an upper portion of the wearer's temporal lobe and a central portion of their cerebellum. In an example, electrodes or other brain activity sensors can collect data on brain activity concerning short term memory and/or hearing. This brain activity data can be associated with selected quantities and types of food consumption and/or can be used to identify quantities and types of food consumed.

In an example, a wearable brain activity monitor can comprise an array of thirteen electrodes or other brain activity sensors which are located substantially at the following set of placement sites—C1, C2, C3, C4, C5, C6, Cz, P7, P8, T7, T8, TP7 and TP8—or which comprise a subset of six or more sites from this set of placement sites.

In an example, a device for measuring and/or modifying a person's food consumption can comprise: a top-and-back loop member which is configured to be worn on a person's head, wherein this top-and-back loop member further comprises a first loop which loops over the top of the person's head and a second loop which loops around the back of the person's head; one or more electrodes or other brain activity sensors which are configured by the top-and-back loop member to be less than one inch from the surface of the person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: a top-and-back loop member which is configured to be worn on a person's head, wherein this top-and-back loop member further comprises a first loop which loops over the top of the person's head and a second loop which loops around the back of the person's head; one or more electrodes or other brain activity sensors which are configured by the top-and-back loop member to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a sensor-positioning member can comprise: (a) two arcuate elements which loop, from side to side, over the central upper portion of the wearer's head and (b) two (left and right side) downward-protruding arcuate elements which terminate in areas behind the wearer's left and right ears, respectively. In an example, a side view of the shape of a sensor-positioning member can look similar to a lower-case letter y.

In an example, an anterior element of two elements which loop over a wearer's head can span an upper portion of a wearer's temporal lobe, a portion of their parietal lobe, and an upper-posterior portion of their cerebral cortex. In an example, electrodes or other brain activity sensors can collect data on brain activity concerning short term memory, smell, taste, hunger, speech, and/or eye movement.

In an example, a posterior element of two elements which loop over a wearer's head can span a laterally-central portion of a wearer's occipital lobe, including the somatosensory area. In an example, down-ward protruding members can span a posterior portion of the wearer's temporal lobe and a laterally-central portion of their cerebellum. In an example, electrodes or other brain activity sensors collect data on brain activity concerning sight, image recognition, speech, object weight, object texture, object temperature, and/or hearing. This brain activity data can be associated with selected quantities and types of food consumption and/or can be used to identify quantities and types of food consumed.

In an example, a wearable brain activity monitor can comprise an array of twenty electrodes or other brain activity sensors which are located substantially at the following set of placement sites—C1, C2, C3, C4, C5, C6, CP1, CP2, CP3, CP4, CP5, CP6, CPz, Cz, P7, P8, T7, T8, TP7 and TP8—or which comprise a subset of ten or more sites from this set of placement sites.

In an example, a sensor-positioning member can comprise: (a) an arcuate ring which encircles the upper portion of the wearer's head; and (b) two (left and right side) downward-protruding arcuate elements which terminate in areas just beneath or just behind the wearer's left and right ears, respectively. In an example, the left-side view of the shape of sensor-positioning member can look similar to a script lower-case Greek letter Tau which has been reflected around its vertical axis. In an example, the arcuate ring can be tilted at an angle with respect to a horizontal plane wherein this angle is in the range of 10 to 40 degrees. In an example, the arcuate ring can be parallel with a horizontal plane as opposed to being tilted. In an example, the arcuate ring can have 3 to 8 sinusoidal oscillations. In an example, the arcuate ring can be a conic section without oscillations. In an example, the downward-protruding elements can curve around the posterior perimeter of the wearer's ears.

In an example, a arcuate ring can span a vertically-central portion of a wearer's occipital lobe, a vertically-central portion of their parietal lobe, their central sulcus, and a portion of their frontal lobe including their cerebral cortex. In an example, electrodes or other brain activity sensors can collect data on brain activity concerning sight, image recognition, hearing and speech, skin sensations, emotions, hunger, and/or higher mental functions. In an example, downward-protruding arcuate elements can span a laterally-central portion of the wearer's occipital lobe, a posterior portion of their temporal lobe, and an anterior portion of their cerebellum. In an example, electrodes or other brain activity sensors can collect data on brain activity concerning speech, short term memory, and/or hearing. This brain activity data can be associated with selected quantities and types of food consumption and/or can be used to identify quantities and types of food consumed.

In an example, a wearable brain activity monitor can comprise an array of eighteen electrodes or other brain activity sensors which are located substantially at the following set of placement sites—AF3, AF4, AFz, C5, C6, CP5, CP6, F5, F6, FC5, FC6, P5, P6, PO3, PO4, POz, TP7 and TP8—or which comprise a subset of eight or more sites from this set of placement sites.

In an example, a sensor-positioning member can comprise: (a) an arcuate element which loops, from the top of one ear to the top of the other ear, around the front-central portion of the wearer's head; (b) two upward-protruding arcuate elements, which rise up from an area behind the wearer's ears and terminate on the right and left sides of their head, respectively, over their occipital lobe; and (c) two downward-protruding arcuate elements, which drop down from an area behind the wearer's ears and terminate in areas below their ears, respectively. In an example, this sensor-positioning member is shaped similar to an eyeglasses frame with the addition of upward protrusions (above the ears) and extended hooking-elements (around the ears).

In an example, an arcuate element which loops around the front-central portion of a wearer's head can span a central portion of the wearer's temporal lobe and their frontal lobe. In an example, electrodes or other brain activity sensors can collect data on brain activity concerning short term memory, smell, taste, and/or higher mental functions. In an example, upward-protruding arcuate elements can span a central portion of the wearer's occipital lobe. In an example, electrodes or other brain activity sensors can collect data on brain activity concerning speech, hearing, and visual processing. In an example, the downward-protruding arcuate elements can span the wearer's cerebellum. In an example, electrodes or other brain activity sensors can collect data on brain activity concerning hearing. This brain activity data can be associated with selected quantities and types of food consumption and/or can be used to identify quantities and types of food consumed.

In an example, a wearable brain activity monitor can comprise an array of twelve electrodes or other brain activity sensors which are located substantially at the following set of placement sites—AF7, AF8, CP5, CP6, F7, F8, FT7, FT8, T7, T8, TP7 and TP8—or which comprise a subset of eight or more sites from this set of placement sites.

In an example, a sensor-positioning member can comprise: (a) an anterior arcuate element which loops from one ear to the other ear, around the front-central portion of the wearer's head; (b) a posterior arcuate element which loops in a curvaceous manner from one ear to the other around the back-central portion of the wearer's head; and (c) two downward-protruding elements which curve around the backs of the wearer's ears and terminate in areas behind (or just below) the ears. In an example, the anterior and/or posterior arcuate elements can be sinusoidal. In an example, the posterior arcuate element can include one or more two or more bends, curves, or loops which span above ear height.

In an example, an anterior arcuate element can span a central portion of a wearer's temporal lobe and a lower portion of their frontal lobe. In an example, electrodes or other brain activity sensors can collect data on brain activity concerning short term memory, smell, taste, and/or higher mental functions. In an example, the posterior arcuate element can span a posterior portion of the wearer's temporal lobe, a central portion of their occipital lobe, and a posterior portion of their occipital lobe, including the visual processing area. In an example, electrodes or other brain activity sensors can collect data on brain activity concerning speech, hearing, vision, and/or image recognition. In an example, two downward-protruding elements can span a posterior portion of the wearer's temporal lobe and an anterior portion of their cerebellum. In an example, electrodes or other brain activity sensors can collect data on brain activity concerning hearing. This brain activity data can be associated with selected quantities and types of food consumption and/or can be used to identify quantities and types of food consumed.

In an example, a wearable brain activity monitor can comprise an array of sixteen electrodes or other brain activity sensors which are located substantially at the following set of placement sites—AF7, AF8, CP5, CP6, F7, F8, FT7, FT8, P5, P6, PO5, PO6, T7, T8, TP7 and TP8—or which comprise a subset of eight or more sites from this set of placement sites.

In an example, a sensor-positioning member can comprise: (a) an upper loop from one ear to the other around the upper-posterior portion of the wearer's head; and (b) a lower loop from one ear to the other around the lower-posterior portion of the wearer's head. In an example, the upper and lower loops can connect at areas just above the wearer's ears. In an example, the average height of the upper loop can be above the average height of the wearer's ears. In an example, the average height of the lower loop can be equal to, or lower than, the average height of the wearer's ears.

In an example, an upper loop can span a portion of a wearer's temporal lobe and a portion of their occipital lobe. In an example, electrodes or other brain activity sensors can collect data on brain activity concerning short term memory, speech, hearing, visual processing, and/or image recognition. In an example, a lower loop can span a portion of the wearer's temporal lobe and a portion of their cerebellum. In an example, electrodes or other brain activity sensors can collect data on brain activity concerning short term memory and hearing. This brain activity data can be associated with selected quantities and types of food consumption and/or can be used to identify quantities and types of food consumed.

In an example, a wearable brain activity monitor can comprise an array of thirteen electrodes or other brain activity sensors which are located substantially at the following set of placement sites—CP5, CP6, P3, P4, P5, P6, PO3, PO4, POz, T7, T8, TP7 and TP8—or which comprise a subset of six or more sites from this set of placement sites.

In an example, a sensor-positioning member can comprise a loop that spans from one ear to the other, looping around the lower-posterior portion of the wearer's head. In an example, the average height of this loop can be equal to, or lower than, the average height of the wearer's ears. In an example, the left-side and right-side ends of the loop can curve around and hook over the tops of the wearer's left and right ears, respectively, terminating in locations just forward of the upper portions of the ears. In an example, a control unit can be just forward of the upper portion of the upper portion of the left ear.

In an example, a loop can span a lower portion of the wearer's temporal lobe and a portion of their cerebellum. In an example, electrodes or other brain activity sensors can collect data on brain activity concerning short term memory, smell, taste, vision and hearing. This brain activity data can be associated with selected quantities and types of food consumption and/or can be used to identify quantities and types of food consumed. In an example, a wearable brain activity monitor can comprise an array of four electrodes or other brain activity sensors which are located substantially at the following set of placement sites—T7, T8, TP7 and TP8—or which comprise a subset of two or more sites from this set of placement sites.

In an example, sensor-positioning member can snuggly loop around a portion of the lateral perimeter of the wearer's ear. In an example, a sensor-positioning member can loop around approximately 70% of the lateral perimeter of the wearer's ear. In various examples, a sensor-positioning member can loop around a percentage of the lateral perimeter of the wearer's ear in the range of 50% to 80%. In an example, the polar coordinates of the lateral perimeter of the wearer's ear can be expressed in terms of positions on a clockface. In an example, sensor positioning member loops from approximately the 10 o'clock position to the 6 o'clock position. In various examples, a sensor-positioning member can loop around the ear within the range of 9 o'clock to 6 o'clock.

In an example, a loop can span a lower portion of a wearer's temporal lobe and a portion of their cerebellum. In an example, electrodes or other brain activity sensors collect data on brain activity concerning short term memory, smell, taste, vision and hearing. This brain activity data can be associated with selected quantities and types of food consumption and/or can be used to identify quantities and types of food consumed.

In an example, a wearable brain activity monitor can comprise an array of four electrodes or other brain activity sensors which are located substantially at the following set of placement sites—T7, T8, TP7 and TP8—or which comprise a subset of two or more sites from this set of placement sites.

In an example, a device for measuring and/or modifying a person's food consumption can comprise: a hearing aid or ear bud which is configured to be worn in, worn on, and/or worn around a person's ear; one or more electrodes or other brain activity sensors which are configured by the hearing aid or ear bud to be less than one inch from the surface of the person's head; a mobile power source and/or power transducer, wherein a power transducer harvests power from human physiological activity and/or environmental energy sources; a data processor; and a data transmitting member.

In an example, a device for measuring and/or modifying a person's food consumption can comprise: a hearing aid or ear bud which is configured to be worn in, worn on, and/or worn around a person's ear; one or more electrodes or other brain activity sensors which are configured by the hearing aid or ear bud to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a sensor-positioning member can snuggly loop around a portion of the lateral perimeter of a wearer's ear and also fit into the wearer's ear canal. In an example, at least one electrode or other brain activity sensor can be within the wearer's ear canal. In an example, a sensor-positioning member can loop around approximately 50% of the lateral perimeter of the wearer's ear. In various examples, a sensor-positioning member can loop around a percentage of the lateral perimeter of the wearer's ear in the range of 25% to 70%. In an example, the polar coordinates of the lateral perimeter of the wearer's ear can be expressed in terms of positions on a clockface. In an example, sensor positioning member loops from approximately the 9 o'clock position to the 3 o'clock position. In various examples, a sensor-positioning member can loop around the ear within the range of 9 o'clock to 5 o'clock.

In an example, a loop can span a lower portion of a wearer's temporal lobe and a portion of their cerebellum. In an example, electrodes or other brain activity sensors can collect data on brain activity concerning short term memory, smell, taste, vision and hearing. This brain activity data can be associated with selected quantities and types of food consumption and/or can be used to identify quantities and types of food consumed. In an example, a wearable brain activity monitor can comprise an array of two electrodes or other brain activity sensors which are located substantially at the following set of placement sites—TP7 and TP8.

In an example, statistical methods can be used to identify specific patterns of electromagnetic brain activity and to identify key associations between food consumption and electromagnetic brain activity. In an example, electromagnetic data from a single EEG electrode at a selected recording place (relative to a reference electrode) can be called an EEG channel. In an example, a statistical method can create a summary statistic or classification for a pattern of data from an EEG channel during a period of time. In various examples, such a summary statistic or classification can be selected from the group consisting of: mean, median, variance, standard deviation, minimum, maximum, frequency, amplitude, and waveform. In various examples, such a summary statistic or classification can be selected from the group consisting of a change following food consumption in one or more of a mean, median, variance, standard deviation, minimum, maximum, frequency, amplitude, and waveform.

In an example, a statistical method can create a summary statistic or classification for data from multiple EEG channels in different recording places during a period of time. In various examples, such a summary statistic can be selected from the group consisting of: the covariance and/or correlation matrix for data from multiple EEG channels; a difference, sum, ratio, product, or other arithmetic function of the mean values of data from multiple EEG channels; a discriminant function of data from multiple EEG channels; a linear function of data from multiple EEG channels; and a non-linear function of data from multiple EEG channels. In various examples, such a summary statistic can be selected from the group consisting of change following food consumption in one or more of: the covariance and/or correlation matrix for data from multiple EEG channels; a difference, sum, ratio, product, or other arithmetic function of the mean values of data from multiple EEG channels; a discriminant function of data from multiple EEG channels; a linear function of data from multiple EEG channels; and a non-linear function of data from multiple EEG channels.

In an example, a statistical method can create a summary statistic or classification for electromagnetic data which repeats within a selected frequency range. In an example, Fourier Transformation can be used. In various examples, such a summary statistic or classification can be selected from the group consisting of: mean, median, variance, standard deviation, minimum, maximum, specific frequency, amplitude, power, and waveform. In various examples, such a summary statistic or classification can be selected from the group consisting of a change following food consumption in one or more of a mean, median, variance, standard deviation, minimum, maximum, specific frequency, amplitude, power, and waveform.

In an example, a statistical method can create a summary statistic or classification for electromagnetic data which repeats within each of multiple frequency ranges. In various examples, such a summary statistic can be selected from the group consisting of: the covariance and/or correlation matrix for data from multiple frequency ranges; a difference, sum, ratio, product, or other arithmetic function of the mean values of data from multiple frequency ranges; a discriminant function of data from multiple frequency ranges; a linear function of data from multiple frequency ranges; and a non-linear function of data from multiple frequency ranges. In various examples, such a summary statistic can be selected from the group consisting of change following food consumption in one or more of: the covariance and/or correlation matrix for data from multiple frequency ranges; a difference, sum, ratio, product, or other arithmetic function of the mean values of data from multiple frequency ranges; a discriminant function of data from multiple frequency ranges; a linear function of data from multiple frequency ranges; and a non-linear function of data from multiple frequency ranges.

In an example, a statistical method can create a summary statistic or classification for electromagnetic data which repeats within each of multiple frequency ranges and varies across multiple recording places. In various examples, such a summary statistic can be selected from the group consisting of: the covariance and/or correlation matrix for data from multiple frequency ranges and multiple recording places; a difference, sum, ratio, product, or other arithmetic function of the mean values of data from multiple frequency ranges and multiple recording places; a discriminant function of data from multiple frequency ranges and multiple recording places; a linear function of data from multiple frequency ranges and multiple recording places; and a non-linear function of data from multiple frequency ranges and multiple recording places. In various examples, such a summary statistic can be selected from the group consisting of change following food consumption in one or more of: the covariance and/or correlation matrix for data from multiple frequency ranges and multiple recording places; a difference, sum, ratio, product, or other arithmetic function of the mean values of data from multiple frequency ranges and multiple recording places; a discriminant function of data from multiple frequency ranges and multiple recording places; a linear function of data from multiple frequency ranges and multiple recording places; and a non-linear function of data from multiple frequency ranges and multiple recording places.

In an example, a wearable EEG monitor can be used to modify a person's food consumption. In an example, a device or system can directly and actively modify a person's electromagnetic brain activity in order to modify their food consumption. In an example, a method, device, or system can modify electromagnetic brain activity directly, rather than relying on the feedback or self-modification of brain activity discussed earlier. In an example, a wearable EEG device may not only measure electromagnetic brain activity, but also directly modify electromagnetic brain activity.

In an example, a wearable EEG device can comprise: a plurality of electrodes or other brain activity; a plurality of electromagnetic transmitters; and a control unit. In an example, the control unit can further comprise a power source, a data processor, and a data transmitter. In an example, a wearable EEG device can comprise electromagnetic transmitters which are separate from electromagnetic sensors. In an example, a set of electrodes can sequentially function as electromagnetic sensors and electromagnetic transmitters. In an example, electromagnetic transmitters can be located at a subset of sites selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, O2, A1 and A2.

In an example, surface electromagnetic patterns (detected at selected locations on the surface of a person's head) can be analyzed to estimate the interior electromagnetic patterns (within particular brain regions) which caused these surface electromagnetic patterns. In an example, a three-dimensional model of the electromagnetic field which created a surface electromagnetic pattern can be created. In an example, this three-dimensional model of the electromagnetic field can then be used to infer the interior electromagnetic pattern which created the surface electromagnetic pattern. In an example, this three-dimensional model of the electromagnetic field can also be used, in reverse, to infer the surface electromagnetic pattern which would be required to recreate the interior electromagnetic pattern.

In an example, a surface electromagnetic pattern which is associated with satiety can be identified. In an example, the interior electromagnetic pattern which is associated with this surface electromagnetic pattern can be determined using a three-dimensional model of the brain's electromagnetic field. In an example, the surface electromagnetic pattern which would be required or recreate an interior electromagnetic pattern associated with satiety can be determined using the three-dimensional model in reverse. Finally, in an example, a person can be given a feeling of satiety by recreating this required surface electromagnetic pattern via an array of electromagnetic transmitters on the surface of their head. In an example, a wearable EEG device has such electromagnetic transmitters and thus have the capability to induce a feeling of satiety.

In an example, a surface electromagnetic pattern which is associated with consumption of good-tasting food can be identified. In an example, the interior electromagnetic pattern which is associated with this surface electromagnetic pattern can be determined using a three-dimensional model of the brain's electromagnetic field. In an example, the surface electromagnetic pattern which would be required to recreate an interior electromagnetic pattern associated with satiety can be determined using the three-dimensional model in reverse. Finally, in an example, a person can be given a eating good-tasting food by recreating this required surface electromagnetic pattern via an array of electromagnetic transmitters on the surface of their head. In an example, this can help a person to enjoy eating healthy food whose taste they would otherwise not enjoy.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using associations between patterns of food consumption and patterns of electromagnetic brain activity in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption during the second time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using associations between patterns of food consumption and patterns of electromagnetic brain activity in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption during the selected time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using the food-brainwave database, wherein this database links patterns of food consumption to patterns of electromagnetic brain activity, in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) providing feedback to the person concerning their estimated food consumption during the second time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using a food-brainwave database, wherein this database links patterns of food consumption to patterns of electromagnetic brain activity, in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) providing feedback to the person concerning their estimated food consumption during the selected time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using associations between patterns of food consumption and patterns of electromagnetic brain activity in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) automatically activating a plurality of electromagnetic transmitters which are less than one inch from the surface of the person's head in response to the person's food consumption from the second time period, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using associations between patterns of food consumption and patterns of electromagnetic brain activity in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) automatically activating a plurality of electromagnetic transmitters which are less than one inch from the surface of the person's head in response to the person's food consumption from the selected time period, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; (e) using the food-brainwave database, wherein this database links patterns of food consumption to patterns of electromagnetic brain activity, in order to estimate the person's food consumption during the second time period from data concerning the person's electromagnetic brain activity from the second time period, wherein food consumption can comprise consumption of food overall or consumption of one or more selected amounts and/or types of foods, ingredients, or nutrients; and (f) automatically activating a plurality of electromagnetic transmitters which are less than one inch from the surface of the person's head in response to the person's food consumption from the second time period, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; (b) using a food-brainwave database, wherein this database links patterns of food consumption to patterns of electromagnetic brain activity, in order to estimate the person's food consumption during the selected time period from data concerning the person's electromagnetic brain activity from the selected time period, wherein food consumption can comprise consumption of food overall or consumption of one or more selected amounts and/or types of foods, ingredients, or nutrients; and (c) automatically activating a plurality of electromagnetic transmitters which are less than one inch from the surface of the person's head in response to the person's food consumption from the selected time period, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) providing feedback to the person concerning their estimated food consumption during the second time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; and (b) providing feedback to the person concerning their estimated food consumption during the selected time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) providing feedback to the person concerning their estimated food consumption during the second time period in order to prompt the person to activate a plurality of electromagnetic transmitters which are less than one inch from the surface of the person's head, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) identifying associations between patterns of food consumption and patterns of electromagnetic brain activity by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) automatically activating a plurality of electromagnetic transmitters which are less than one inch from the surface of the person's head in response to the person's food consumption from the second time period, wherein activation of these electromagnetic transmitters modifies the person's electromagnetic brain activity to a pattern which is associated with satiety.

In an example, a method for modifying a person's food consumption can comprise: (a) receiving data concerning a person's electromagnetic brain activity from a selected time period from one or more electrodes or other brain activity sensors which are configured to be less than one inch from the surface of the person's head; and (b) automatically activating a plurality of electromagnetic transmitters which are less than one inch from the surface of the person's head in response to the person's food consumption from the selected time period, wherein activation of these electromagnetic transmitters modifies the person's electromagnetic brain activity to a pattern which is associated with satiety.

In an example, a method for modifying a person's food consumption can comprise: (a) receiving data concerning a person's food consumption from a first time period: wherein this data is selected from the group consisting of: data communicated by the person via a touch screen interface, speech recognition interface, motion recognition interface, gesture recognition interface, eye movement interface, EMG recognition interface, or keyboard, keypad, or buttons, data from analysis of food images, food packaging, or food labels, data from a spectroscopic food probe, data from a smart food utensil, data from one or more wearable cameras, data from one or more motion sensors, data from one or more electromagnetic sensors in electromagnetic communication with the person's mouth, nose, tongue, esophagus, stomach, intestine or in electromagnetic communication with a nerve which innervates the person's mouth, nose, tongue, esophagus, stomach, or intestine, data from one or more optical sensors in optical communication with the person's mouth, nose, tongue, esophagus, stomach, intestine, or circulatory system, and data from one or more chemical sensors in fluid communication with the person's mouth, nose, tongue, esophagus, stomach, intestine, or circulatory system; and wherein food consumption can comprise consumption of food overall or consumption of one or more selected amounts and/or types of foods, ingredients, or nutrients; (b) receiving data concerning the person's electromagnetic brain activity from the first time period from one or more electrodes or other brain activity sensors which are configured to be less than one inch from the surface of the person's head; (c) creating a food-brainwave database, wherein this database links patterns of food consumption to patterns of electromagnetic brain activity, by analyzing data concerning the person's food consumption and data concerning the person's electromagnetic brain activity from the first time period; (d) receiving data concerning the person's electromagnetic brain activity from a second time period from one or more electrodes or other brain activity sensors which are configured to be less than one inch from the surface of the person's head; and (e) automatically activating a plurality of electromagnetic transmitters which are less than one inch from the surface of the person's head in response to the person's food consumption from the second time period, wherein activation of these electromagnetic transmitters modifies the person's electromagnetic brain activity to a pattern which is associated with satiety.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and an accelerometer. In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; an accelerometer; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a blood chemistry sensor.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a blood chemistry sensor; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a heart rate monitor. In an example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a heart rate monitor; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; and a plurality of electromagnetic signal transmitters which are configured to be less than one inch from the surface of a person's head, wherein these electromagnetic signal transmitters collectively modify an electromagnetic field in order to reproduce a pattern of brain activity which is associated with satiety and/or consumption of a specific type of food, ingredient, or nutrient.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a data transmitter; a plurality of electromagnetic signal transmitters which are configured to be less than one inch from the surface of a person's head, wherein these electromagnetic signal transmitters collectively modify an electromagnetic field in order to reproduce a pattern of brain activity which is associated with satiety and/or consumption of a specific type of food, ingredient, or nutrient; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; and a tactile interface for computer-to-human communication.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; a tactile interface for computer-to-human communication; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; and an olfactory interface for computer-to-human communication.

In another example, a device for measuring and/or modifying a person's food consumption can comprise: a plurality of electrodes or other brain activity sensors which are configured to be worn less than one inch from the surface of a person's head; a mobile power source and/or power transducer, wherein a power transducer harvests power from human physiological activity and/or environmental energy sources; a data processor; an olfactory interface for computer-to-human communication; and a database which includes associations between specific patterns of food consumption and specific patterns of electromagnetic brain activity, which translates specific types and amounts of food into specific types and amounts of nutrients, or which does both.

In another example, a device for measuring and/or modifying a person's food consumption can comprise: a cap, beanie, hat, or helmet which is configured to be worn on a person's head; one or more electrodes or other brain activity sensors which are configured by the cap, beanie, hat, or helmet to be less than one inch from the surface of the person's head; a mobile power source and/or power transducer, wherein a power transducer harvests power from human physiological activity and/or environmental energy sources; a data processor; and a data transmitting member.

In another example, a device for measuring and/or modifying a person's food consumption can comprise: a cap, beanie, hat, or helmet which is configured to be worn on a person's head; one or more electrodes or other brain activity sensors which are configured by the cap, beanie, hat, or helmet to be less than one inch from the surface of the person's head; a mobile power source and/or power transducer, wherein a power transducer harvests power from human physiological activity and/or environmental energy sources; a data processor; a data transmitter; and a database which includes associations between specific patterns of food consumption and specific patterns of electromagnetic brain activity, which translates specific types and amounts of food into specific types and amounts of nutrients, or which does both.

In an example, a method to modify a person's food consumption can comprise: (a) receiving data concerning a person's food consumption and data concerning the person's brain activity from a first time period; (b) identifying associations between food consumption patterns and brain activity patterns based on data from the first time period; (c) receiving data concerning the person's brain activity from a second time period; (d) estimating the person's food consumption from the person's brain activity from the second time period using the associations which were previously identified.

In an example, a wearable device to modify a person's food consumption can comprise: (a) a plurality of electromagnetic sensors worn by a person on their head, wherein these electromagnetic sensors measure changes in the person's brain activity caused by food consumption, and wherein these changes in brain activity are then used to estimate the person's food consumption; (b) a power source; (c) a data processor; (d) a data transmitter; and (e) a user interface, wherein this interface provides feedback to the person concerning their estimated food consumption.

In an example, a wearable device to modify a person's food consumption can comprise: (a) a plurality of electromagnetic sensors worn by a person on their head, wherein these electromagnetic sensors measure changes in the person's brain activity caused by food consumption, and wherein these changes in brain activity are then used to estimate the person's food consumption; (b) a power source; (c) a data processor; (d) a data transmitter; and (e) a user interface, wherein this interface provides feedback to the person concerning their brain activity patterns to help the person self-modify their brain activity pattern to a pattern that is associated with satiety.

In an example, a wearable device to modify a person's food consumption can comprise: (a) a plurality of electromagnetic sensors worn by a person on their head, wherein these electromagnetic sensors measure changes in the person's brain activity caused by food consumption; (b) a plurality of electromagnetic transmitters worn by the person on their head, wherein these electromagnetic transmitters modify the person's brain activity pattern to a pattern associated with satiety; (c) a power source; (d) a data processor; and (e) a data transmitter.

In an example, a system to modify a person's food consumption can comprise: (a) a brain activity sensing device which is worn by a person on their head, wherein this device further comprises a plurality of electromagnetic sensors which measure changes in the person's brain activity caused by food consumption, wherein these changes are used to estimate the person's food consumption; a power source; a data processor; and a data transmitter; and (b) a user interface device with which the brain activity sensing device is in wireless communication, wherein this interface device provides feedback to the person concerning their estimated food consumption.

In an example, a system to modify a person's food consumption can comprise: (a) a brain activity sensing device which is worn by a person on their head, wherein this device further comprises a plurality of electromagnetic sensors which measure changes in the person's brain activity caused by food consumption, wherein these changes are used to estimate the person's food consumption; a power source; a data processor; and a data transmitter; and (b) a user interface device with which the brain activity sensing device is in wireless communication, wherein this interface device provides feedback to the person concerning their brain activity patterns to help the person self-modify their brain activity pattern to a pattern that is associated with satiety.

In an example, a device can comprise eyewear whose optical properties are controlled by changes in a wearer's electromagnetic brain activity. In an example, this eyewear can modify a person's view of their environment based on changes in their brain activity. This eyewear can enable a person to control characteristics of their visual perception of their environment by changing their brainwaves. In various examples, this eyewear can comprise lenses or other light-transmitting members whose light absorption, light reflection, light refraction, light spectrum transformation, focal direction, focal distance, light polarization, or parallax view can be controlled by the wearer's brain activity.

In an example, a device can be embodied in eyewear with an integrated camera or other imaging component whose operation is controlled by changes in the wearer's electromagnetic brain activity. In a simple example, a camera can be turned on or off based on changes in the person's brain activity. In other examples, this eyewear can enable a person to control various parameters of how a camera records the environment. In an example, a camera's focal direction, focal distance, spectral filter, image retention, or image transmission can be controlled by a person via changes in their brain activity.

In an example, a device can be embodied in eyewear that transmits and/or displays a combination of environmental objects and virtual objects. In an example, a person can modify and control the combination of environmental and virtual objects which they see by changing their brainwave patterns. In an example, a person can alter the relative proportion of environmental content vs. virtual content in an augmented reality system based on changes in their electromagnetic brain activity. In an example, a person can alter and control the type of virtual content which is combined with environmental content by changing their brain activity. In various examples, a device can be embodied in a type of eyewear selected from the group consisting of: non-prescription eyeglasses, prescription eyeglasses, sunglasses, goggles, contact lenses, visor, monocle, eyewear-based human-to-computer interface, eyeglasses with integrated camera, augmented reality (AR) glasses, and virtual reality (VR) glasses.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; and (c) a data control unit. In an example, a data control unit can further comprise a power source, a data processor, and a data transmitter and/or receiver.

In an example, an electromagnetic energy sensor can be an electrode. In an example, an electromagnetic energy sensor can be an electroencephalogram (EEG) electrode. In an example, an electromagnetic energy sensor can be a dry electrode. In an example, an electromagnetic energy sensor can measure electromagnetic brain activity and/or brainwaves. In an example, an electromagnetic sensor can be located within an inch of the surface of a person's head. In an example, an electromagnetic sensor can be in direct contact with the surface of a person's head. In an example, electromagnetic energy data can be recorded at a rate in the range of 100 to 500 samples per second.

In an example, electromagnetic brain activity data from a single electromagnetic energy sensor (relative to a reference place) can be called a channel. In an example, electromagnetic brain activity data from multiple electromagnetic energy sensors can be called a montage. In various examples, one or more electromagnetic energy sensors can be configured at locations selected from the group of electrode sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, one or more reference sites can be selected from the group of sites consisting of A1 and A2.

In an example, a light-transmitting optical member can be a lens. In an example, a lens can be one of two lenses in a pair of eyeglasses. In an example, a lens can be a contact lens. In an example, a lens can be located in goggles or a visor. In an example, a lens or other light-transmitting optical member can be made from a material whose optical attributes are changed by application of an electrical current. In an example, a lens can be a compound lens. In an example, a lens or other light-transmitting optical member can be made from multiple components whose combined optical attributes are changed by application of an electrical current. In an example, a lens or other light-transmitting optical member can have one or more optoelectronic or photoelectric components. In an example, a lens or other light-transmitting optical member can be a variable lens with a fluid component.

In an example, a light-transmitting optical member can be shaped such that its optical attributes are changed by its overall movement relative to a person's eye. In an example, a light-transmitting optical member can also comprise one or more actuators to move it or to move components within it. In an example, a light-transmitting optical member can have multiple components such that its optical attributes are changed by the movement of a first set of components relative to a second set of components. In an example, a lens or other light-transmitting optical member can comprise an array of micro-lenses or micro-mirrors. In an example, the absorption, reflection, refraction, polarization, or collimation of light through a light-transmitting optical member can be changed by application of electrical current to the member. In an example, the absorption, reflection, refraction, polarization, or collimation of light through a light-transmitting optical member can be changed by the overall movement of the member or by movement of a first set of components in an optical member relative to a second set of components in the optical member. In an example, a light-transmitting optical member or multiple components within such a member can be moved by a mechanism selected from the group consisting of: electric motor, piezoelectric actuator, Micro Electro Mechanical System (MEMS), and micro motor.

In an example, a light-transmitting optical member can be further comprised of one or more components selected from the group consisting of: simple lens, concave lens, concentric lenses, convex lens, diverging lens, asymmetric lens, compound lens, fly's eye lens, Fresnel lens, light-transducing element, microlens array, microspheres, optoelectronic lens, parabolic lens, wedge-shaped lens, liquid crystal, liquid lens, Digital Micromirror Device (DMD), Digital Light Processor (DLP), Electromagnetically Induced Transparency (EIT) structure, Liquid Crystal Display (LCD), MEMS-based lens array, MEMS-based mirror array, birefringent material, carbon nanotube, light-guiding metamaterial structure, light-guiding metamaterial structure, light-guiding tubes, metamaterial light channel, microscale glass beads, nanorods, nanoscale gratings, nanotubes, etched waveguide, nanoimprint lithography pathways, resonant grating filter, Split Ring Resonators (SRRs), thermoplastic nanoimprint pathways, crystal, crystal array, crystalline structures, photonic metamaterial, photonic crystal, fiber optics, optical fiber, polarizing filter, cylindrical prism, prism, wedge prism, acrylic mirror, concentric reflective surfaces, dielectric mirror, parabolic mirror, reflector array, and retroreflective structure.

In an example, a data control unit can comprise: a power source or transducer; and a data processor. In an example, a power source can be a battery. In various examples, a data control unit can further comprise one or more components selected from the group consisting of: a wireless data transmitter; a wireless data reception component; a data memory component; a computer-to-human interface; and a human-to-computer interface. In an example, a power source or transducer can further comprise: power from a source that is internal to the device during regular operation (such as an internal battery, capacitor, energy-storing microchip, wound coil or spring); power that is obtained, harvested, or transduced from a source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion, electromagnetic energy from the person's body, or thermal energy from the person's body).

In an example, a data control unit can be in direct electrical communication with one or more electromagnetic energy sensors by wires or other electrically-conductive pathways. In an example, a data control unit can be in wireless communication with one or more electromagnetic energy sensors. In an example, a data control unit can be in direct electrical communication with one or more light-transmitting members by wires or other electrically-conductive pathways. In an example, a data control unit can be in wireless communication with one or more light-transmitting members.

In an example, a data control unit can be in wireless communication with a separate wearable device selected from the group consisting of: a wristwatch, smart watch, fitness watch, watch phone, bracelet phone, smart bracelet, fitness bracelet, smart wrist band, electronically-functional wrist band, other wrist-worn electronic device, or smart armband; a smart button, electronically-functional button, pin, brooch, pendant, beads, neck chain, necklace, dog tags, locket, or medallion; a smart finger ring, electronically-functional finger ring, electronically-functional earring, nose ring, or ear bud or clip; a wearable camera; an article of smart clothing, an electronically-functional shirt, electronically-functional pants, or a smart belt.

In an example, a data control unit can be in wireless communication with a separate mobile device selected from the group consisting of: smart phone, mobile phone, holophone, or cellular phone; PDA; electronic tablet; electronic pad; and other electronically-functional handheld device. In an example, a data control unit can be in wireless communication with a relatively fixed-location device selected from the group consisting of: laptop computer, desktop computer, internet terminal, smart appliance, home control system, and other fixed-location electronic communication device.

In an example, a device can be embodied in eyewear that modifies visual perception based on electromagnetic energy measured from a person's head, comprising: (a) one or more electrodes configured to be within three inches of the surface of a person's head which measure electromagnetic energy from the person's head; (b) one or more lenses configured to be within three inches of the surface of the person's head and configured to transmit light into one or both of the person's eyes; (c) a power source or transducer; and (d) a data processor or transmitter.

In an example, electromagnetic energy data from one or more electromagnetic energy sensors can be statistically analyzed in order to identify significant patterns and/or changes in a person's electromagnetic brain activity. These significant patterns and/or changes in brain activity can then be used to control the transmission of light through one or more light-transmitting optical members in eyewear. In an example, a device can comprise eyewear whose optical transmission attributes are modified by changes in a person's brainwaves. In various examples, the absorption, reflection, refraction, polarization, or parallax view of light through one or more light-transmitting optical members can be modified by a person's electromagnetic brain activity. In various examples, the focal distance, view direction, and/or view scope of images transmitted through one or more light-transmitting optical members can be modified by a person's electromagnetic brain activity. In various examples, the spectrum of light absorbed, transmitted, or shifted through the one or more light-transmitting optical members can be modified by a person's brain activity.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which modifies transmitted light in a first manner and a second configuration which modifies transmitted light in a second manner, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein transmitted light is modified based on a change in data from the one or more of the electromagnetic energy sensors; and (c) a data control unit that further comprises a power source and a data processor.

In various examples, one or more primary statistical methods can be used to identify specific patterns in a person's electromagnetic brain activity and/or specific changes in the person's electromagnetic brain activity. In an example, data from one or more electromagnetic sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter.

In an example, a pattern and/or change in electromagnetic brain activity can be a one-time pattern. In another example, a pattern of electromagnetic brain activity can repeat over time in a rhythmic manner. In an example, a primary statistical method can analyze repeating electromagnetic patterns by analyzing their frequency of repetition, their frequency band or range of repetition, their recurring amplitude, their wave phase, and/or their waveform. In an example repeating patterns and/or waveforms can be analyzed using Fourier Transform methods.

In an example, a primary statistical method for identifying patterns and/or changes in electromagnetic brain activity can comprise finding the mean or average value of data from one or more brain activity channels during a period of time. In an example, a statistical method can comprise identifying a significant change in the mean or average value of data from one or more brain activity channels. In an example, a statistical method can comprise finding the median value of data from one or more brain activity channels during a period of time. In an example, a statistical method can comprise identifying a significant change in the median value of data from one or more brain activity channels. In an example, a statistical method can comprise identifying significant changes in the relative mean or median data values among multiple brain activity channels. In an example, a statistical method can comprise identifying significant changes in mean data values from a first set of electrode locations relative to mean data values from a second set of electrode locations. In an example, a statistical method can comprise identifying significant changes in mean data recorded from a first region of the brain relative to mean data recorded from a second region of the brain.

In an example, a primary statistical method for identifying patterns and/or changes in electromagnetic brain activity can comprise finding the minimum or maximum value of data from one or more brain activity channels during a period of time. In an example, a statistical method can comprise identifying a significant change in the minimum or maximum value of data from one or more brain activity channels. In an example, a statistical method can comprise identifying significant changes in the relative minimum or maximum data values among multiple brain activity channels. In an example, a statistical method can comprise identifying significant changes in minimum or maximum data values from a first set of electrode locations relative to minimum or maximum data values from a second set of electrode locations. In an example, a statistical method can comprise identifying significant changes in minimum or maximum data values recorded from a first region of the brain relative to minimum or maximum data values recorded from a second region of the brain.

In an example, a primary statistical method for identifying patterns and/or changes in electromagnetic brain activity can comprise finding the variance or the standard deviation of data from one or more brain activity channels during a period of time. In an example, a statistical method can comprise identifying a significant change in the variance or the standard deviation of data from one or more brain activity channels. In an example, a statistical method can comprise identifying significant changes in the covariation and/or correlation among data from multiple brain activity channels. In an example, a statistical method can comprise identifying significant changes in the covariation or correlation between data from a first set of electrode locations relative and data from a second set of electrode locations. In an example, a statistical method can comprise identifying significant changes in the covariation or correlation of data values recorded from a first region of the brain and a second region of the brain.

In an example, a primary statistical method for identifying patterns and/or changes in electromagnetic brain activity can comprise finding the amplitude of waveform data from one or more channels during a period of time. In an example, a statistical method can comprise identifying a significant change in the amplitude of waveform data from one or more channels. In an example, a statistical method can comprise identifying significant changes in the relative wave amplitudes from one or more channels. In an example, a statistical method can comprise identifying significant changes in the amplitude of electromagnetic signals recorded from a first region of the brain relative to the amplitude of electromagnetic signals recorded from a second region of the brain.

In an example, a primary statistical method for identifying patterns and/or changes in electromagnetic brain activity can comprise finding the power of waveform brain activity data from one or more channels during a period of time. In an example, a statistical method can comprise identifying a significant change in the power of waveform data from one or more channels. In an example, a statistical method can comprise identifying significant changes in the relative power levels of one or more channels. In an example, a statistical method can comprise identifying significant changes in the power of electromagnetic signals recorded from a first region of the brain relative to the power of electromagnetic signals recorded from a second region of the brain.

In an example, a primary statistical method for identifying patterns and/or changes in electromagnetic brain activity can comprise finding a frequency or a frequency band of waveform and/or rhythmic brain activity data from one or more channels which repeats over time. In an example, Fourier Transform methods can be used to find a frequency or a frequency band of waveform and/or rhythmic data which repeats over time. In an example, a statistical method can comprise decomposing a complex waveform into a combination of simpler waveforms which each repeat at a different frequency or within a different frequency band. In an example, Fourier Transform methods can be used to decomposing a complex waveform into a combination of simpler waveforms which each repeat at a different frequency or within a different frequency band.

In an example, a primary statistical method for identifying patterns and/or changes in electromagnetic brain activity can comprise identifying significant changes in the amplitude, power level, phase, frequency, covariation, entropy, and/or oscillation of waveform data from one or more channels. In an example, a statistical method can comprise identifying significant changes in the amplitude, power level, phase, frequency, covariation, entropy, and/or oscillation of waveform data within a selected frequency band. In an example, a statistical method can comprise identifying significant changes in the relative amplitudes, power levels, phases, frequencies, covariations, entropies, and/or oscillations of waveform data among different frequency bands. In various examples, these significant changes can be identified using Fourier Transform methods.

In an example, brainwaves or other rhythmic, cyclical, and/or repeating electromagnetic signals associated with brain activity can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier Transformation methods. In an example, brainwaves can be measured and analyzed using a subset and/or combination of five clinical frequency bands: Delta, Theta, Alpha, Beta, and Gamma. In an example, a method can analyze changes in brainwaves in a single frequency band, changes in brainwaves in multiple frequency bands, or changes in brainwaves in a first frequency band relative to those in a second frequency band.

In an example, Delta brainwaves can be measured and analyzed within a frequency band of 1 to 4 Hz. In various examples, Delta brainwaves or other rhythmic, cyclical, and/or repeating electromagnetic signals associated with brain activity can be measured and analyzed within a frequency band selected from the group consisting of: 0.5-3.5 Hz, 0.5-4 Hz, 1-3 Hz, 1-4 Hz, and 2-4 Hz. In an example, a method can track a decrease or increase in the relative power of brainwaves in the Delta band. In an example, a method can track a frequency shift within the Delta frequency band. In an example, a method can track a change in wave shape for brainwaves in the Delta frequency band. In an example, a method can track a change in which brain regions originate or modify brainwaves within the Delta frequency band. In an example, a method can track a change in brainwave activity within the Delta band from the anterior vs. posterior areas of a person's brain. In an example, a method can track a change in brainwave activity within the Delta band for a particular brain lobe or organelle. In an example, a method can track a change in brainwave activity within the Delta band as measured from a specific electrode site, a specific electrode channel, and/or a specific montage of channels.

In an example, Theta brainwaves can be measured and analyzed within a frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz. In an example, a method can track changes in the power of brainwaves in the Theta band. In an example, a method can track a frequency shift within the Theta band. In an example, a method can track changes in wave shape for brainwaves in the Theta band. In an example, a method can track a change in which brain regions originate or modify brainwaves within the Theta band. In an example, a method can track a change in brainwave activity within the Theta band as measured from a specific electrode site, a specific electrode channel, and/or a specific montage of channels.

In an example, Alpha brainwaves can be measured and analyzed within a frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, a method can track an increase or decrease in the relative power of brainwaves in the Alpha band. In an example, a method can track a downward or upward shift in the frequency of brainwaves within the Alpha band. In an example, a method can track a change in wave shape for brainwaves in the Alpha frequency band. In an example, a method can track a change in which brain regions originate or modify brainwaves within the Alpha frequency band. In an example, a method can track a change in brainwave activity within the Alpha band on one side of a person's brain relative to the other side. In an example, a method can track a change in brainwave activity within the Alpha band in a particular brain lobe or organelle. In an example, a method can track a change in brainwave activity within the Alpha band as measured from a specific electrode site, a specific electrode channel, and/or a specific montage of channels.

In an example, Beta brainwaves can be measured and analyzed within a frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, specific patterns or trends in brainwaves in the Beta frequency band can be statistically identified.

In an example, Gamma brainwaves can be measured and analyzed within a frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz. In an example, specific patterns or trends in brainwaves in the Gamma frequency band can be statistically identified. In an example, a person can be identified as having the World's Best Gamma and receive an appropriately-labeled coffee mug.

In an example, a primary statistical method can employ multivariate analysis of electromagnetic brainwave activity in the Delta, Theta, and Alpha frequency bands to identify patterns. In an example, a primary statistical method can comprise calculating an arithmetic function, or a change in an arithmetic function, of the different power levels in multiple frequency bands. In an example, a primary statistical method can comprise a difference, or a change in a difference, between power levels in different frequency bands. In an example, a primary statistical method can comprise a ratio, or a change in a ratio, of power levels in different frequency bands. In an example, a primary statistical method can comprise a sum, or a change in a sum, of power levels in different frequency bands. In an example, a primary statistical method can comprise a product, or a change in a product, of power levels in different frequency bands.

In various examples, specific patterns of electromagnetic brain activity can be analyzed and identified using one or more statistical methods selected from the group consisting of: ANOVA or MANOVA; artificial neural network; auto-regression; Bonferroni analysis; centroid analysis; chi-squared analysis; cluster analysis and grouping; decision tree or random forest analysis; Discrete Fourier transform (DFT), Fast Fourier Transform (FFT), or other Fourier Transform methods; factor analysis; feature vector analysis; fuzzy logic model; Gaussian model; hidden Markov model, input-output hidden Markov model, or other Markov model; inter-band mean; inter-band ratio; inter-channel mean; inter-channel ratio; inter-montage mean; inter-montage ratio; Kalman filter; kernel estimation; linear discriminant analysis; linear transform; logit model; AI (e.g. machine learning); mean power; mean; median; multi-band covariance analysis; multi-channel covariance analysis; multivariate linear regression or multivariate least squares estimation; multivariate logit or other multivariate parametric classifiers; naive Bayes classifier, trained Bayes classifier, dynamic Bayesian network, or other Bayesian methods; non-linear programming; pattern recognition; power spectral density or other power spectrum analysis; principal components analysis; probit model; support vector machine; time-series model; T-test; variance, covariance, or correlation; waveform identification; multi-resolution wavelet analysis or other wavelet analysis; whole band power; and Z-scores or other data normalization method.

In an example, one or more electromagnetic energy sensors can measure electromagnetic activity concerning eye movements and/or muscle activity. In an example, an electromagnetic energy sensor can be an electrooculography (EOG) sensor or an electromyography (EMG) sensor. In an example, the optical attributes of electronically-functional eyewear can be controlled by changes in eye movements as measured by one or more EOG sensors. In an example, one or more EOG sensors can be integrated into a portion of eyewear which spans the front of a person's face. In an example, the optical attributes of electronically-functional eyewear can be controlled by changes in muscle activity as measured by one or more EMG sensors. In an example, a device can be embodied in eyewear which comprises one or more electromagnetic energy sensors selected from the group consisting of: EOG sensors which measure electromagnetic activity concerning eye movements; EMG sensors which measure electromagnetic activity concerning muscle activity, and EEG sensors which measure electromagnetic brain activity.

In an example, a device can be embodied in brainwave-controlled sunglasses. In an example, a device can comprise sunglasses or other eyewear which partially block light transmission, wherein the amount of light blocked or transmitted can be modified by changes in the person's brain activity. In an example, a device can comprise sunglasses whose opacity and/or reflectivity is controlled by changes in brain activity by the person wearing them. In an example, a device can enable a person to increase or decrease the transparency or opacity of their eyewear by changing their brainwave activity. In an example, such brainwave-controlled sunglasses can be useful for protecting a person's eyes from intensive light sources. In an example, such brainwave-controlled sunglasses can be useful for maintaining a person's privacy. In an alternative example, the electromagnetic sensors of this device can measure electromagnetic signals from eye movements instead of brain activity. In an alternative example, this device can enable a person to increase or decrease the transparency or opacity of their eyewear by moving their eyes in a selected direction or motion pattern.

In an example, a person can change the transparency of lenses or other light-transmitting members by changing the power of their brainwaves in the Delta frequency band. In an example, a person can change the transparency of lenses or other light-transmitting members by changing the power of their brainwaves in the Theta band. In an example, a person can change the transparency of lenses or other light-transmitting members by changing the power of their brainwaves in the Alpha frequency band. In an example, a person can change the transparency of lenses or other light-transmitting members by changing the power of their brainwaves in the Beta frequency band. In an example, a person can change the transparency of lenses or other light-transmitting members by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which absorbs a first amount of light and a second configuration which absorbs a second amount of light, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors configured to be within three inches of the surface of a person's head and configured to measure electromagnetic energy from the person's head; one or more light-transmitting optical members configured to be within three inches of the surface of the person's head and configured to transmit light into one or both of the person's eyes, wherein one or more of the light-transmitting optical members has a first configuration which absorbs a first amount of light and a second configuration which absorbs a second amount of light, and wherein one or more light-transmitting optical members are changed from the first configuration to the second configuration, or vice versa, based on data from one or more of the electromagnetic energy sensors; and a data control unit.

In an example, in a first configuration a light-transmitting member can transmit a plurality of (e.g. two) light rays. In a second configuration the light-transmitting member can transmit only a portion of this plurality (e.g. one) of these light rays. In the second configuration, some light rays are absorbed in response to the measurement and identification of electromagnetic brain activity. In an example, a light-transmitting member can be a lens whose transparency changes due to application of an electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the amount of light that is absorbed by one or more light-transmitting optical members is modified based on a change in data from the one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which reflects a first amount of light and a second configuration which reflects a second amount of light, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors configured to be within three inches of the surface of a person's head and configured to measure electromagnetic energy from the person's head; one or more light-transmitting optical members configured to be within three inches of the surface of the person's head and configured to transmit light into one or both of the person's eyes, wherein one or more of the light-transmitting optical members has a first configuration which reflects a first amount of light and a second configuration which reflects a second amount of light, and wherein one or more light-transmitting optical members are changed from the first configuration to the second configuration, or vice versa, based on data from one or more of the electromagnetic energy sensors; and a data control unit.

In an example, in a first configuration a light-transmitting member transmits light rays. In the second configuration the light-transmitting member reflects light rays. In an example, light-transmitting member can be a lens whose reflectivity is changed by application of an electrical current or a moveable micro-mirror array.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the amount of light that is reflected by one or more light-transmitting optical members is modified based on a change in data from the one or more of the electromagnetic energy sensors; and (c) a data control unit that further comprises a power source and a data processor.

In an example, a device can comprise eyewear in which changes in a person's view angle, view direction, or breadth of view are controlled by changes in the person's electromagnetic brain activity. In an example, this device can enable a person to change the scope or breadth of their field of vision by changing their brainwave pattern. In an example, this device can enable a person to directly see objects in their peripheral field of vision by changing their brainwave pattern. In an example, this device can enable a person to selectively adjust the polar coordinate (around the circumference of their head) of their field of vision through the eyewear. In an example, this device can enable a person to change their perspective to see behind them, wherein this change in perspective is controlled by a change in their brainwave patterns.

In an example, a person can change their view direction and/or the field of vision through lenses or other light-transmitting members by changing the power of their brainwaves in the Delta frequency band. In an example, a person can change their view direction and/or field of vision through lenses or other light-transmitting members by changing the power of their brainwaves in the Theta band. In an example, a person can change their view direction and/or field of vision through lenses or other light-transmitting members by changing the power of their brainwaves in the Alpha frequency band. In an example, a person can change their view direction and/or field of vision through lenses or other light-transmitting members by changing the power of their brainwaves in the Beta frequency band. In an example, a person can change their view direction and/or field of vision through lenses or other light-transmitting members by changing the power of their brainwaves in the Gamma band.

In an example, a device can be embodied in eyewear which modifies the appearance of a person's eyes as seen by others through a lens or other light-transmitting member. In an example, this change in appearance can be achieved through a selective change in the refraction of light through a central portion of the light-transmitting member. In an example, this change in appearance can be achieved through a selective change in the concavity or convexity of a central portion of a light-transmitting member. In an example, a person can modify and control the appearance of their pupils to other people, in real time, by changing their brain activity. In an example, a person can modify the apparent size or shape of their pupils by changing their brain activity. In an example, a person can increase or decrease the apparent dilation of their pupils by changing their brainwave patterns. In an example, a person can increase the apparent dilation of their pupils to others in order to convey greater excitement or interest.

In an example, a person can change the apparent dilation of their pupils through lenses or other light-transmitting members by changing the power of their brainwaves in the Delta frequency band. In an example, a person can change the apparent dilation of their pupils through lenses or other light-transmitting members by changing the power of their brainwaves in the Theta band. In an example, a person can change the apparent dilation of their pupils through lenses or other light-transmitting members by changing the power of their brainwaves in the Alpha frequency band. In an example, a person can change the apparent dilation of their pupils through lenses or other light-transmitting members by changing the power of their brainwaves in the Beta frequency band. In an example, a person can change the apparent dilation of their pupils through lenses or other light-transmitting members by changing the power of their brainwaves in the Gamma band.

In an example, a person can change the apparent dilation of their pupils through lenses or other light-transmitting members by changing the specific frequency of their brainwaves in the Delta frequency band. In an example, a person can change the apparent dilation of their pupils through lenses or other light-transmitting members by changing the specific frequency of their brainwaves in the Theta band. In an example, a person can change the apparent dilation of their pupils through lenses or other light-transmitting members by changing the specific frequency of their brainwaves in the Alpha frequency band. In an example, a person can change the apparent dilation of their pupils through lenses or other light-transmitting members by changing the specific frequency of their brainwaves in the Beta frequency band. In an example, a person can change the apparent dilation of their pupils through lenses or other light-transmitting members by changing the specific frequency of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which refracts light rays to a first exit angle and a second configuration which refracts light rays to a second exit angle, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors configured to be within three inches of the surface of a person's head and configured to measure electromagnetic energy from the person's head; one or more light-transmitting optical members configured to be within three inches of the surface of the person's head and configured to transmit light into one or both of the person's eyes, wherein one or more of the light-transmitting optical members has a first configuration which refracts light rays to a first exit angle and a second configuration which refracts light rays to a second exit angle, and wherein one or more light-transmitting optical members are changed from the first configuration to the second configuration, or vice versa, based on data from one or more of the electromagnetic energy sensors; and a data control unit.

In an example, in a first configuration, a light-transmitting member can transmit and/or refract light rays to a first exit angle. In a second configuration, the light-transmitting member can transmit and/or refract light rays to a second exit angle. In an example, a light-transmitting member can be a lens, prism, or lens array whose surface angles are changed by application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the exit angle of light rays refracted by one or more light-transmitting optical members is modified based on a change in data from the one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which refracts light rays from a first entrance angle and a second configuration which refracts light rays from a second entrance angle, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the entrance angle of light rays refracted by one or more light-transmitting optical members is modified based on a change in data from the one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration through which other people see the person's pupils as having a first size or dilation and a second configuration through which other people see the person's pupils as having a second size or dilation, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration through which other people see the person's pupils as having a first size or dilation and a second configuration through which other people see the person's pupils as having a second size or dilation, and wherein one or more light-transmitting optical members are changed from the first configuration to the second configuration, or vice versa, based on data from one or more of the electromagnetic energy sensors; and a data control unit.

In an example, in a first configuration, a light-transmitting member refracts light rays so that a person's pupil appears to have a first size. In an example, in a second configuration, the light-transmitting member refracts light rays so that the person's pupil appears to have a second size. In an example, the light-transmitting member can be a lens or lens array whose concavity or convexity is changed by the application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which transmits light from a first view direction and a second configuration which transmits light from a second view direction, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration which transmits light from a first view direction and a second configuration which transmits light from a second view direction, and wherein one or more light-transmitting optical members are changed from the first configuration to the second configuration, or vice versa, based on data from one or more of the electromagnetic energy sensors; and a data control unit.

In an example, in a first configuration, a light-transmitting member can transmit and/or refract light rays so that the person sees in a first view direction. In a second configuration, the light-transmitting member transmits and/or refracts light rays so that the person sees in a second direction. In an example, a light-transmitting member can be a prism, lens, or lens array whose surface angles are changed by the application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the view direction that is transmitted by one or more light-transmitting optical members is modified based on a change in data from the one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which transmits light from a first view scope and/or breadth and a second configuration which transmits light from a second view scope and/or breadth, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration which transmits light from a first view scope and/or breadth and a second configuration which transmits light from a second view scope and/or breadth, and wherein one or more light-transmitting optical members are changed from the first configuration to the second configuration, or vice versa, based on data from one or more of the electromagnetic energy sensors; and a data control unit.

In an example, in a first configuration, a light-transmitting member can transmit and/or refract light rays so that the person sees with a first view scope and/or breadth. In a second configuration, the light-transmitting member transmits and/or refracts light rays so that the person sees with a second view scope and/or breadth. In an example, a light-transmitting member can be a lens or lens array whose concavity or convexity is changed by the application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the view scope and/or breath that is transmitted by one or more light-transmitting optical members is modified based on a change in data from one or more of the electromagnetic energy sensors; and (c) a data control unit that further comprises a power source and a data processor.

In an example, a device can be embodied in eyewear with a variable focal distance, wherein this focal distance can be modified by a change in a person's electromagnetic brain activity. In an example, this device can enable a person to focus on environmental objects at difference distances by changing their brainwave patterns. In an example, a device can comprise thought-controlled bifocal eyewear wherein the same set of lenses can focus on a distant object or on a nearby object, depending on a person's brainwave pattern. In an example, a device can comprise thought-controlled binoculars, wherein the same set of lenses can focus on a normal-distance object or a far-distance object depending on a person's brainwave pattern.

In an example, a person can change their focal distance through lenses or other light-transmitting members by changing the power of their brainwaves in the Delta frequency band. In an example, a person can change their focal distance through lenses or other light-transmitting members by changing the power of their brainwaves in the Theta band. In an example, a person can change their focal distance through lenses or other light-transmitting members by changing the power of their brainwaves in the Alpha frequency band. In an example, a person can change their focal distance through lenses or other light-transmitting members by changing the power of their brainwaves in the Beta frequency band. In an example, a person can change their focal distance through lenses or other light-transmitting members by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration with a first focal distance and a second configuration with a second focal distance, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration with a first focal distance and a second configuration with a second focal distance, and wherein one or more light-transmitting optical members are changed from the first configuration to the second configuration, or vice versa, based on data from one or more of the electromagnetic energy sensors; and a data control.

In an example, in a first configuration, a light-transmitting member can transmit and/or refract light rays so that the person sees with a first focal distance. In a second configuration, the light-transmitting member transmits and/or refracts light rays so that the person sees with a second focal distance. In an example, a light-transmitting member can be a variable-focal-length lens or lens array whose focal length is changed by the application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the focal distance of one or more light-transmitting optical members is modified based on a change in data from one or more of the electromagnetic energy sensors; and (c) a data control unit that further comprises a power source and a data processor.

In an example, a device can be embodied in eyewear which filters, blocks, amplifies, shifts, and/or analyzes light in one or more bands or ranges within the light spectrum, wherein this modification of light spectrum is controlled by changes in a person's electromagnetic brain activity. In an example, a person can automatically change the color and/or tint of lenses in a pair of glasses by changing their brainwave pattern. In an example, a person can literally see the world through rosy-tinted glasses whenever they figuratively see the world through rosy-tinted glasses.

In an example, a device can be embodied in eyewear which modifies the appearance of a person's eyes to others through a lens or other light-transmitting member. In an example, a person can modify and control the appearance of their pupils to other people, in real time, by changing their electromagnetic brain activity. In an example, a person can change the tint of lenses or other light-transmitting members in real time by changing their electromagnetic brain activity. In an example, a person can change the apparent color of their pupils to red when they are upset. In an example, a person can change the apparent color of their pupils to blue when they are calm.

In an example, a person can automatically filter out light energy in a selected band or range of the spectrum by changing their brainwave pattern. In an example, a person can shift light in a portion of the light spectrum which is transmitted through eyewear based on changes in their brainwave pattern. In an example, a person can extend their vision into the lower or upper non-visible ranges of the light spectrum by shifting light upward or downward. In an example, a person can adjust eyewear by changing their brainwave activity so that infrared light energy becomes visible to them through the eyewear. In an example, a person can adjust eyewear by changing their brainwave activity so that ultraviolet light energy becomes visible to them through the eyewear. In an example, a person can activate spectroscopic analysis of light energy, such as by using Fourier Transform, by changing their electromagnetic brain activity. In an example, a person can use such eyewear to activate spectral analysis of objects within their field of vision to obtain information about the chemical composition of such subjects.

In an example, a person can change the spectral distribution of light seen through lenses or other light-transmitting members by changing the power of their brainwaves in the Delta frequency band. In an example, a person can change the spectral distribution of light seen through lenses or other light-transmitting members by changing the power of their brainwaves in the Theta band. In an example, a person can change the spectral distribution of light seen through lenses or other light-transmitting members by changing the power of their brainwaves in the Alpha frequency band. In an example, a person can change the spectral distribution of light seen through lenses or other light-transmitting members by changing the power of their brainwaves in the Beta frequency band. In an example, a person can change the spectral distribution of light seen through lenses or other light-transmitting members by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which filters and/or absorbs light in a portion of the light spectrum by a first amount and a second configuration which filters and/or absorbs light in a portion of the light spectrum by a second amount, wherein the first amount can be zero, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration which filters and/or absorbs light in a portion of the light spectrum by a first amount and a second configuration which filters and/or absorbs light in a portion of the light spectrum by a second amount, wherein the first amount can be zero, and wherein one or more light-transmitting optical members are changed from the first configuration to the second configuration, or vice versa, based on data from one or more of the electromagnetic energy sensors; and a data control unit.

In an example, in a first configuration a light-transmitting member can transmit a first light ray in a first portion of the light spectrum and second light ray in a second portion of the light spectrum. In a second configuration, the light-transmitting member transmits only the first light ray. The second light ray is filtered out. In an example, a light-transmitting member can be a lens or a lens array with a variable spectral filter which is changed by the application of electrical current.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the amount by which one or more light-transmitting optical members filters and/or absorbs light in a portion of the light spectrum is modified based on a change in data from one or more of the electromagnetic energy sensors; and (c) a data control unit that further comprises a power source and a data processor.

In an example, a person can change the apparent color of their eyes as seen by others through lenses or other light-transmitting members by changing the power of their brainwaves in the Delta frequency band. In an example, a person can change the apparent color of their eyes as seen by others through lenses or other light-transmitting members by changing the power of their brainwaves in the Theta band. In an example, a person can change the apparent color of their eyes as seen by others through lenses or other light-transmitting members by changing the power of their brainwaves in the Alpha frequency band. In an example, a person can change the apparent color of their eyes as seen by others through lenses or other light-transmitting members by changing the power of their brainwaves in the Beta frequency band. In an example, a person can change the apparent color of their eyes as seen by others through lenses or other light-transmitting members by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration through which other people see the person's pupils as having a first color and a second configuration through which other people see the person's pupils as having a second color, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which shifts the wavelength of light in a portion of the light spectrum by a first amount and a second configuration which shifts the wavelength of light in a portion of the light spectrum by a second amount, wherein the first amount can be zero, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes, wherein one or more of the light-transmitting optical members has a first configuration which shifts the wavelength of light in a portion of the light spectrum by a first amount and a second configuration which shifts the wavelength of light in a portion of the light spectrum by a second amount, wherein the first amount can be zero, and wherein one or more light-transmitting optical members are changed from the first configuration to the second configuration, or vice versa, based on data from one or more of the electromagnetic energy sensors; and a data control unit.

In a first configuration, a light-transmitting member can transmit light rays without changing their wavelengths. In a second configuration, a light-transmitting member shifts the wavelengths of the light rays.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the amount by which one or more light-transmitting optical members shifts the wavelength of light in a portion of the light spectrum is modified based on a change in data from one or more of the electromagnetic energy sensors; and (c) a data control unit that further comprises a power source and a data processor.

In an example, a device can be embodied in eyewear which polarizes transmitted light in response to a change in a person's electromagnetic brain activity. In an example, being able to selectively activate light polarization can enable a person to selectively view selected 3D images in three dimensions or in two dimensions. In an example, a person can change the polarity of light seen through lenses or other light-transmitting members by changing the power of their brainwaves in the Delta frequency band. In an example, a person can change the polarity of light seen through lenses or other light-transmitting members by changing the power of their brainwaves in the Theta band. In an example, a person can change the polarity of light seen through lenses or other light-transmitting members by changing the power of their brainwaves in the Alpha frequency band. In an example, a person can change the polarity of light seen through lenses or other light-transmitting members by changing the power of their brainwaves in the Beta frequency band. In an example, a person can change the polarity of light seen through lenses or other light-transmitting members by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein one or more of the light-transmitting optical members has a first configuration which polarizes light by a first amount and a second configuration which polarizes light by a second amount, wherein the first amount can be zero, and wherein one or more light-transmitting optical members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes, wherein the amount by which one or more light-transmitting optical members polarizes light is modified based on a change in data from one or more of the electromagnetic energy sensors; and (c) a data control unit that further comprises a power source and a data processor.

In an example, a device can be embodied in a system for private viewing of a computer display screen comprising: (a) a computer display screen which displays content in a selected range of the light spectrum which is not normally visible to the human eye; (b) one or more electromagnetic energy sensors which are configured to be within three inches of the surface of a person's head and measure electromagnetic energy from the person's head; and (c) eyewear with one or more light-transmitting members, wherein these one or more light-transmitting members have a first configuration which does not modify transmitted light in the selected spectral range so that it becomes visible to the human eye and a second configuration which does modify transmitted light in the selected spectral range so that it becomes visible to the human eye, and wherein one or more light-transmitting members are changed from the first configuration to the second configuration by changes in the person's electromagnetic brain activity. In an example, a selected range of light which the computer screen displays and the selected range which the eyewear displays can vary in synchronization with each other, so that only a person wearing the eyewear can see the content displayed on the computer screen.

In an example, a device can be embodied in a system for private viewing of a computer display screen comprising: a computer display screen which displays content in a selected range of the light spectrum which is not normally visible to the human eye; one or more electromagnetic energy sensors within three inches of the surface of a person's head and measure electromagnetic energy from the person's head; and eyewear with one or more light-transmitting members, wherein these one or more light-transmitting members have a first configuration which does not modify transmitted light in the selected spectral range so that it becomes visible to the human eye and a second configuration which does modify transmitted light in the selected spectral range so that it becomes visible to the human eye, and wherein one or more light-transmitting members are changed from the first configuration to the second configuration by changes in the person's electromagnetic brain activity. These figures also show a data control unit.

In a first configuration, a light-transmitting member can transmit light rays without changing their wavelengths, wherein the content on a computer display screen is not visible. In a second configuration, the light-transmitting member can shift the wavelengths of light rays so that the content on the computer display screen is visible.

In an example, a device can be embodied in a system for private viewing of a computer display screen comprising: (a) a computer display screen which displays content which requires polarized lenses to be visible to the human eye; (b) one or more electromagnetic energy sensors which are configured to be within three inches of the surface of a person's head and measure electromagnetic energy from the person's head; and (c) eyewear with light-transmitting members, wherein these light-transmitting members have a first configuration with a first amount or direction of polarization and a second configuration with a second amount or direction of polarization, wherein the first amount can be zero, and wherein one or more light-transmitting members are changed from the first configuration to the second configuration by changes in the person's electromagnetic brain activity. In an example, the polarization of content that computer screen displays and a polarization filter of eyewear displays can vary in synchronization with each other, so that only a person wearing the eyewear can see the content displayed on the computer screen.

In an example, a device can comprise a specific type and/or shape of wearable eyewear frame. In an example, a wearable frame can hold electromagnetic sensors, light-transmitting members, and a data control unit in a selected configuration. In an example, a wearable frame can position one or more light-transmitting members in front of a person's eyes similar to the way in which a conventional pair of eyeglasses positions one or more lenses in front of a person's eyes. In an example, a wearable frame can be adjustable to enable adjustment of the configuration of electromagnetic sensors, light-transmitting members, and/or a data control unit. In an example, adjustment can be manual. In an example, adjustment can be done by one or more actuators. In an example, adjustment can be automated and iterative based on a specific person's anatomy and physiology.

In an example, a wearable eyewear frame can hold one or more electromagnetic sensors in contact with the surface of a person's head. In an example, one or more electromagnetic sensors can be spring-loaded to maintain compressive contact with the person's head. In an example, the degree of compressive contact can be adjusted. In an example, this adjustment can be manual. In an example, this adjustment can be automated to ensure proper electromagnetic contact and communication. In an example, eyewear can further comprise one or more actuators which maintain proper contact pressure between one or more electromagnetic sensors and the surface of the person's head.

In an example, a wearable eyewear frame can hold one or more electromagnetic sensors in contact with one or both sides of a person's head. In an example, a wearable frame can hold one or more electromagnetic sensors in contact in one or more locations between a person's ear and eye. In an example, a wearable frame can hold one or more electromagnetic sensors in contact with a person's forehead. In an example, a wearable frame can be similar to the frame of conventional eyeglasses, except that it has a central upward bulge, loop, or projection which holds an electrode in contact with a person's forehead. In an example, an upward bulge, loop, or projection can extend upwards from the bridge of a person's nose to cover a portion of the person's forehead. In an example, an upward bulge, loop, or projection can arc over a portion of the person's forehead from a person's left eyebrow to the person's right eyebrow. In an example, an upward bulge, loop, or projection can arc over a portion of the person's forehead from a person's left temple to the person's right temple.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective positions, including one or more positions on the person's forehead. In an example, an electromagnetic energy sensor can be held in contact with a person's forehead by an upward bulge, loop, or projection from the wearable frame.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective positions, including one or more positions on the person's forehead. In an example, an electromagnetic energy sensor can be held in contact with a person's forehead by an upward bulge, loop, or projection from wearable frame.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is configured to span from the person's left ear to the person's face, then span across the front of the person's face including a portion of the person's nose, and then span from the person's face to the person's right ear.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is configured to span from the person's left ear to the person's face, then span across the front of the person's face including a portion of the person's forehead, and then span from the person's face to the person's right ear.

In an example, a device can comprise a wearable eyewear frame that is similar to a conventional eyeglasses frame with the addition of an arcuate member from one ear to the other which spans a portion of the person's forehead. In an example, a device can comprise a wearable frame with a left side piece between the left ear and left eye, with a right side piece between the right ear and right eye, and an arcuate member spanning from the left side piece to the right side piece which covers a portion of the person's forehead. In an example, an arcuate member can hold one or more electromagnetic sensors in contact with the person's forehead.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective positions, wherein this wearable frame further comprises a left-side member spanning from the person's left ear to their face, a right-side member spanning from the person's right ear to their face, and an arcuate member which connects the left-side member to the right-side member and spans the person's forehead.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit that further comprises a power source and a data processor; and a wearable frame which holds the one or more electromagnetic energy sensors and the one or more light-transmitting optical members in their respective positions, wherein this wearable frame further comprises a left-side member spanning from the person's left ear to their face, a right-side member spanning from the person's right ear to their face, and an arcuate member which connects the left-side member to the right-side member and spans the person's forehead.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame has a first member which is configured to span from one of the person's ears to the person's nose and a second member which is configured to span from one of the person's ears to the person's forehead.

In an example, a device can be embodied in eyewear that measures electromagnetic energy from a person's head, comprising: one or more electromagnetic energy sensors within three inches of the surface of a person's head and configured to measure electromagnetic energy from the person's head, wherein at least one of the electromagnetic energy sensors is configured to measure electromagnetic energy from the person's forehead; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations, wherein this wearable frame has a first member which is configured to span from one of the person's ears to the person's nose and a second member which is configured to span from one of the person's ears to the person's forehead.

In an example, a device can be embodied in eyewear with a wearable frame which is similar to the frame of conventional eyeglasses except that it also includes an arcuate member or extension which loops around the rear of the person's head. In an example, this rearward-looping arcuate member or extension can hold additional electromagnetic sensors against the surface of the person's head to improve the scope and accuracy of brain activity measurement. This can be particularly useful for measuring activity from the person's occipital lobe or cerebellum. Further, this rearward-looping arcuate member or extension can also help to hold the device on the person's head if it is heavier than ordinary eyeglasses.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is configured to span from the person's left ear to the person's face, then span across the front of the person's face, then span from the person's face to the person's right ear, and then span from the person's right ear to the person's left ear by looping around the rear of the person's head.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit that further comprises a power source and a data processor; and a wearable frame which holds the one or more electromagnetic energy sensors and the one or more light-transmitting optical members in their respective configurations, wherein this wearable frame is configured to span from the person's left ear to the person's face, then span across the front of the person's face, then span from the person's face to the person's right ear, and then span from the person's right ear to the person's left ear by looping around the rear of the person's head.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit that further comprises a power source and a data processor; and a wearable frame which holds the one or more electromagnetic energy sensors and the one or more light-transmitting optical members in their respective configurations, wherein this wearable frame is configured to span from the person's left ear to the person's face, then span across the front of the person's face, then span from the person's face to the person's right ear, and then span from the person's right ear to the person's left ear by looping over the top of the person's head.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame has a first portion which is configured to span from the person's left ear to the person's face, has a second portion which is configured to span across the front of the person's face, has a third portion which is configured to span from the person's face to the person's right ear, has a fourth portion which is configured to span from the person's right ear to the person's left ear by looping around the rear of the person's head, and has a fifth portion which is configured to span from the person's right ear to the person's left ear by looping over the top of the person's head.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations, wherein this wearable frame has a first portion which is configured to span from the person's left ear to the person's face, has a second portion which is configured to span across the front of the person's face, has a third portion which is configured to span from the person's face to the person's right ear, has a fourth portion which is configured to span from the person's right ear to the person's left ear by looping around the rear of the person's head, and has a fifth portion which is configured to span from the person's right ear to the person's left ear by looping over the top of the person's head.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations; wherein this wearable frame is configured to curve around the anterior perimeter of the person's left ear, then span from the person's left ear to the person's face, then span across the front of the person's face, then span from the person's face to the person's right ear, and then curve around the anterior perimeter of the person's right ear; and wherein one or more of the electromagnetic energy sensors are located along a portion of the wearable frame which curves around the anterior perimeter of an ear.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations; wherein this wearable frame is configured to curve around the anterior perimeter of the person's left ear, then span from the person's left ear to the person's face, then span across the front of the person's face, then span from the person's face to the person's right ear, and then curve around the anterior perimeter of the person's right ear; and wherein one or more of the electromagnetic energy sensors are located along a portion of the wearable frame which curves around the anterior perimeter of an ear.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations; wherein this wearable frame is configured to curve around the posterior perimeter of the person's left ear, then span from the person's left ear to the person's face, then span across the front of the person's face, then span from the person's face to the person's right ear, and then curve around the posterior perimeter of the person's right ear; and wherein one or more of the electromagnetic energy sensors are located along a portion of the wearable frame which curves around the posterior perimeter of an ear.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations; wherein this wearable frame is configured to curve around the posterior perimeter of the person's left ear, then span from the person's left ear to the person's face, then span across the front of the person's face, then span from the person's face to the person's right ear, and then curve around the posterior perimeter of the person's right ear; and wherein one or more of the electromagnetic energy sensors are located along a portion of the wearable frame which curves around the posterior perimeter of an ear.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is substantially circular or elliptical and wherein this wearable frame spans both the person's forehead and the rear of the person's head.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations, wherein this wearable frame is substantially circular or elliptical and wherein this wearable frame spans both the person's forehead and the rear of the person's head.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is substantially circular or elliptical and wherein this wearable frame encircles the person's head at an anterior acute angle in the range of 0 to 45 degrees with respect to a horizontal plane when the person's head is upright.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is substantially sinusoidal in shape and wherein this wearable frame spans both the person's forehead and the rear of the person's head.

In an example, a device can be embodied in eyewear that measures electromagnetic energy from a person's head, comprising: one or more electromagnetic energy sensors within three inches of the surface of a person's head and configured to measure electromagnetic energy from the person's head; one or more light-transmitting optical members within three inches of the surface of the person's head and configured to transmit light into one or both of the person's eyes; a data control unit; and a wearable frame which holds the one or more electromagnetic energy sensors and the one or more light-transmitting optical members in their respective configurations, wherein this wearable frame is substantially sinusoidal in shape and wherein this wearable frame spans both the person's forehead and the rear of the person's head.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit that further comprises a power source and a data processor; and (d) a wearable frame which holds the one or more electromagnetic energy sensors and the one or more light-transmitting optical members in their respective configurations, wherein this wearable frame is substantially sinusoidal in shape and wherein the central axis of sinusoidal undulations encircles the person's head at an anterior acute angle in the range of 0 to 45 degrees with respect to a horizontal plane when the person's head is upright.

In an example, a device can be embodied in eyewear that measures electromagnetic energy from a person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head and configured to transmit light into one or both of the person's eyes; (c) a data control unit; and (d) a wearable frame which holds the one or more electromagnetic energy sensors and the one or more light-transmitting optical members in their respective configurations, wherein this wearable frame is substantially the shape of an ellipse projected downward onto the curvature of the person's head and wherein this wearable frame spans both the person's forehead and the rear of the person's head.

In an example, a device can be embodied in eyewear whose appearance to other people is modified based on electromagnetic energy measured from a person's head, comprising: (a) one or more electromagnetic energy sensors configured to be within three inches of the surface of a person's head and configured to measure electromagnetic energy from the person's head; (b) one or more light-emitting members configured to be within three inches of the surface of the person's head; and (c) a data control unit. In an example, an electromagnetic energy sensor can be an electrode. In an example, a light-emitting member can be selected from the group consisting of: Light Emitting Diode (LED), infrared (IR) light source, laser, ultraviolet (UV) light source, Liquid Crystal Display (LCD), photoluminescent light source, Electro Luminescent (EL) light source. In an example, a data control unit can further comprise a power source and a data processor.

In an example, eyewear can include one or more LEDs or other light-emitting members whose light intensities and/or colors change when a person's electromagnetic brain activity changes. In an example, an eyewear frame can include one or more LEDs wherein different color LEDs light up with increases in the power of different brainwave frequency bands. In an example, different frequency bands selected from the group consisting of Delta, Theta, Alpha, Beta, and Gamma can each be associated with a different color. In an example, the overall color of light emitted from the eyewear can change with changes in the relative power of brainwaves in the Delta, Theta, Alpha, Beta, and/or Gamma frequency bands.

In an example, the spectrum of light emitted from eyewear can change with changes electromagnetic brain activity. In an example, an increase in brainwave activity in the Delta band can cause an increase in light emitted from eyewear in a first frequency range. In an example, an increase in brainwave activity in the Theta band can cause an increase in light emitted from eyewear in a second frequency range. In an example, an increase in brainwave activity in the Alpha band can cause an increase in light emitted from eyewear in a third frequency range. In an example, an increase in brainwave activity in the Beta band can cause an increase in light emitted from eyewear in a third frequency range. In an example, an increase in brainwave activity in the Gamma band can cause an increase in light emitted from eyewear in a third frequency range.

In an example, the device disclosed herein can enable a person to control the intensity, color, spectrum, polarization, and/or collimation of light emitted from eyewear by changing their electromagnetic brain activity. In an example, thought-controlled changes in the intensity, color, and/or spectrum of light emitted from eyewear can be useful for non-verbal communication. In an example, changes in the intensity, color, and/or spectrum of light emitted from eyewear can be useful for maintaining a person's privacy and/or disrupting unwelcome photography. In an example, a person can cause eyewear to emit infrared light energy by changing their brainwaves to a selected pattern. In an example, the emission of infrared light energy can be useful for disrupting unwelcome photography by a proximal imaging device.

In an example, a person can cause eyewear to emit a pulse of high-intensity light energy in the visual spectrum. In an example, a pulse of high-energy light in the visual spectrum can be useful for disrupting unwelcome photography by a nearby imaging device. In an example, a brainwave-controlled pulse of high-energy light in the visual spectrum can serve a safety or emergency purpose. In an example, an unexpected pulse of high-energy light can temporarily blind an attacker.

In an example, a device can be embodied in eyewear whose appearance is modified based on electromagnetic energy measured from a person's head, comprising: (a) one or more electromagnetic energy sensors configured to be within three inches of the surface of a person's head and configured to measure electromagnetic energy from the person's head; (b) one or more light-emitting members configured to be within three inches of the surface of the person's head, wherein one or more light-emitting members have a first configuration which emits light with a first intensity and a second configuration which emits light with a second intensity, wherein the first intensity can be zero, and wherein the one or more light-emitting members are changed from the first configuration to the second configuration by changes in electromagnetic energy from the person's head; and (c) a data control unit.

In an example, a device can be embodied in eyewear whose appearance is modified based on electromagnetic energy measured from a person's head, comprising: one or more electromagnetic energy sensors within three inches of the surface of a person's head and configured to measure electromagnetic energy from the person's head; one or more light-emitting members within three inches of the surface of the person's head, wherein one or more light-emitting members have a first configuration which emits light with a first intensity and a second configuration which emits light with a second intensity, wherein the first intensity can be zero, and wherein the one or more light-emitting members are changed from the first configuration to the second configuration by changes in electromagnetic energy from the person's head; and a data control unit. In an example, LEDs can be turned on by the identification of a specific pattern of electromagnetic brain activity which is measured by electromagnetic energy sensors.

In an example, a device can be embodied in eyewear whose appearance is modified based on electromagnetic energy measured from a person's head, comprising: (a) one or more electromagnetic energy sensors configured to be within three inches of the surface of a person's head and configured to measure electromagnetic energy from the person's head; (b) one or more light-emitting members configured to be within three inches of the surface of the person's head, wherein one or more light-emitting members have a first configuration which emits light with a first spectral distribution and a second configuration which emits light with a second spectral distribution, and wherein the one or more light-emitting members are changed from the first configuration to the second configuration by changes in electromagnetic energy from the person's head; and (c) a data control unit.

In an example, a device can be embodied in eyewear whose appearance is modified based on electromagnetic energy measured from a person's head, comprising: one or more electromagnetic energy sensors within three inches of the surface of a person's head and configured to measure electromagnetic energy from the person's head; one or more light-emitting members within three inches of the surface of the person's head, wherein one or more light-emitting members have a first configuration which emits light with a first spectral distribution and a second configuration which emits light with a second spectral distribution, and wherein the one or more light-emitting members are changed from the first configuration to the second configuration by changes in electromagnetic energy from the person's head; and a data control unit. In an example, the color of an LED can be changed based on a pattern of electromagnetic brain activity which is measured by electromagnetic energy sensors.

In an example, a device can be embodied in eyewear whose appearance is modified based on electromagnetic energy measured from a person's head, comprising: (a) one or more electromagnetic energy sensors configured to be within three inches of the surface of a person's head and configured to measure electromagnetic energy from the person's head; (b) one or more light-emitting members configured to be within three inches of the surface of the person's head, wherein one or more light-emitting members have a first configuration which emits a first amount of infrared light and a second configuration which emits a second amount of infrared light, and wherein the one or more light-emitting members are changed from the first configuration to the second configuration by changes in electromagnetic energy from the person's head; and (c) a data control unit.

In an example, a device can be embodied in eyewear which includes one or more near-eye display screens or other image-display members wherein the image displayed can be changed by a change in a person's electromagnetic brain activity. In an example, eyewear can include one or more display screens or other image-display members which can display virtual objects, environmental objects, or a mixture of virtual and environmental objects. In an example, eyewear can include one or more display screens or other image-display members which display environmental objects from different directions or perspectives.

In an example, a display screen or other image-display member can span an upper portion of a person's field of vision. In an example, a display screen or other image-display member can span a side or peripheral portion of a person's field of vision. In an example, an image-display member can be integrated into a lens or other image-transmitting member. In an example, an image-display member can be separate from a lens or other image-transmitting member. In an example, an image-display member can be held in a position near a person's eye by an eyewear frame in a manner similar to the way in which a lens is held near a person's eye by a conventional eyeglasses frame. In an example, eyewear can hold two display screens in positions which are close to a person's eyes. In an example, one or more display screens or other image-display members can be integrated into one or more contact lenses.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; and (c) a data control unit. In an example, a data control unit can further comprises a power source, a data processor, and a data transmitter.

In an example, an electromagnetic energy sensor can be an electrode. In an example, an electromagnetic energy sensor can measure electromagnetic brain activity. In an example, an electromagnetic energy sensor can be an electroencephalogram (EEG) electrode. In various examples, one or more electromagnetic energy sensors can be configured at locations selected from the group of electrode sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, one or more reference sites can be selected from the group of sites consisting of A1 and A2. In an example, brainwave-controlled eyewear can be part of the Internet of Thinks (IOT).

In an example, an image-display member can be a transparent or translucent near-eye display screen. In an example, an image-display member can be a near-eye image projection surface. In an example, an image-display member can be a portion of a generally-transparent member, such as a lens, wherein only this portion displays a virtual image rather than light transmitted directly from the environment. In an example, an image-display member can superimpose an image of a virtual object on a view of an environmental object. In an example, an image-display member can comprise an augmented reality device or system. In an example, an image-display member can be a composite member which partially displays virtual content and partially transmits light from environmental objects. In an example, an image-display member can be co-located with a light-transmitting member. In an example, an image-display member can be adjacent to a light-transmitting member. In an example, an image-display member can be an optical member with variable transparency and/or variable display brightness, wherein the level of transparency or brightness can be controlled by changes in a person's electromagnetic brain activity.

In an example, an image-display member can comprise one or more components selected from the group consisting of: near-eye display screen, compound lens and display screen, lens with nanoscale gratings, Light Emitting Diode (LED), infrared (IR) light source, laser, ultraviolet (UV) light source, Liquid Crystal Display (LCD), photoluminescent light source, Electro Luminescent (EL) light source, incandescent light source, simple lens, concave lens, concentric lenses, convex lens, diverging lens, asymmetric lens, compound lens, fly's eye lens, Fresnel lens, light-transducing element, microlens array, microspheres, optoelectronic lens, parabolic lens, wedge-shaped lens, liquid crystal, liquid lens, Digital Micromirror Device (DMD), Digital Light Processor (DLP), Electromagnetically Induced Transparency (EIT) structure, Liquid Crystal Display (LCD), MEMS-based lens array, MEMS-based mirror array, Holovision™ display, birefringent material, carbon nanotube, light-guiding metamaterial structure, light-guiding metamaterial structure, light-guiding tubes, metamaterial light channel, microscale glass beads, nanorods, nanoscale gratings, nanotubes, etched waveguide, nanoimprint lithography pathways, resonant grating filter, Split Ring Resonators (SRRs), thermoplastic nanoimprint pathways, crystal, crystal array, crystalline structures, photonic metamaterial, photonic crystal, fiber optics, optical fiber, polarizing filter, cylindrical prism, prism, wedge prism, acrylic mirror, concentric reflective surfaces, dielectric mirror, parabolic mirror, reflector array, and retroreflective structure.

In an example, a device can be embodied in eyewear that modifies visual perception based on electromagnetic energy measured from a person's head, comprising: (a) one or more electrodes configured to be within three inches of the surface of a person's head which measure electromagnetic energy from the person's head; (b) one or more near-eye display screens configured to be within three inches of the surface of the person's head; (c) a power source or transducer; and (d) a data processor or transmitter.

In an example, a device can be embodied in eyewear that modifies visual perception based on electromagnetic energy measured from a person's head, comprising: (a) one or more electrodes configured to be within three inches of the surface of a person's head which measure electromagnetic energy from the person's head; (b) one or more lenses with virtual display capability which are configured to be within three inches of the surface of the person's head; (c) a power source or transducer; and (d) a data processor or transmitter.

In an example, electromagnetic energy data that is measured by one or more electromagnetic energy sensors can be statistically analyzed in order to identify significant patterns and/or changes in a person's electromagnetic brain activity. Significant changes in brain activity can then be used to control changes in the images which are displayed and/or transmitted by one or more image-display members. In an example, a device can comprise eyewear whose image display attributes are modified by changes in a person's brainwaves. In an example, one or more image-display members can be turned on or off by changes in a person's electromagnetic brain activity. In an example, the brightness of one or more image-display members can be adjusted by changes in a person's electromagnetic brain activity. In an example, the virtual content displayed by one or more image-display members can be adjusted by changes in a person's electromagnetic brain activity. In an example, the mix of virtual vs. environmental objects displayed by one or more image-display members can be adjusted by changes in a person's electromagnetic brain activity.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members have a first configuration which shows a first image and a second configuration which shows a second image, and wherein one or more image-display members are changed from the first configuration to the second configuration, or vice versa, based on data from one or more of the electromagnetic energy sensors; and (c) a data control unit that further comprises a power source and a data processor.

In various examples, one or more primary statistical methods can be used to identify specific patterns of electromagnetic brain activity and/or specific changes in electromagnetic brain activity. In an example, data from one or more electromagnetic sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In an example, a pattern or change in electromagnetic brain activity may be a one-time pattern and/or change. In an example, a pattern of electromagnetic brain activity can repeat over time in a rhythmic manner. In an example, a primary statistical method can analyze repeating electromagnetic patterns by analyzing their frequency of repetition, their frequency band or range of repetition, their recurring amplitude, their wave phase, and/or their waveform. In an example, repeating patterns and/or waveforms can be analyzed using Fourier Transform methods.

In an example, a device can comprise eyewear with an image-display member wherein the brightness or size of the image displayed by the image-display member is modified by changes in the person's electromagnetic brain activity. In an example, an image-display member can be modified from having a very dim or small (perhaps even non-visible) image to have a very bright or large (highly visible) image by changes in a person's electromagnetic brain activity. In an example, a device can comprise eyewear with an image-display member wherein the brightness or size of the image displayed by the image-display member is controlled by electromagnetic (EMG or EOG) signals from muscles moving the person's eyes. In an example, a person can adjust an image-display member from having a very dim or small (even non-visible) image to have a very bright or large (highly visible) image by moving their eyes.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays images with a first brightness level and a second configuration which displays images with a second brightness level, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more image-display members configured to be within three inches of the person's head, wherein one or more of the image-display members has a first configuration which displays images with a first brightness level and a second configuration which displays images with a second brightness level, and wherein one or more image-display members are changed from the first configuration to the second configuration, or vice versa, based on data from one or more of the electromagnetic energy sensors; and a data control unit.

In a first configuration, an image-display member can display light rays comprising a virtual object with a first brightness level and display and/or transmit light rays of real objects in the person's environment. In a second configuration, the image-display member can display light rays comprising a virtual object with a second (higher) brightness level and continue to display and/or transmit light rays of real objects in the person's environment.

In an example, a person can change the brightness of an image displayed by an image-display member by changing the power of their brainwaves in the Delta frequency band. In an example, a person can change the brightness of an image displayed by an image-display member by changing the power of their brainwaves in the Theta band. In an example, a person can change the brightness of an image displayed by an image-display member by changing the power of their brainwaves in the Alpha frequency band. In an example, a person can change the brightness of an image displayed by an image-display member by changing the power of their brainwaves in the Beta frequency band. In an example, a person can change the brightness of an image displayed by an image-display member by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays images in a first display size and a second configuration which displays images in a second display size, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays images in a first display location (relative to an eye) and a second configuration which displays images in a second display location (relative to an eye), and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear wherein the person wearing the eyewear sees the environment from different angles or perspectives based on changes in their brain activity. In an example, the eyewear changes a person's view angle, view direction, or breadth of view based on changes in their brain activity. In an example, a person can control the scope or breadth of their field of vision by changing their brainwave pattern. In an example, the person can see objects in the periphery of their field of vision, or even behind them, by changing their brainwave pattern. In an example, eyewear embodied by this device can enable a person to rotate their angle of vision around the circumference of their head (such as represented by polar coordinates or clockface coordinates) by changing a parameter of their electromagnetic brain activity.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays a view of the environment from a first direction and a second configuration which displays a view of the environment from a second direction, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more image-display members within three inches of the person's head, wherein one or more of the image-display members has a first configuration which displays a view of the environment from a first direction and a second configuration which displays a view of the environment from a second direction, and wherein one or more image-display members are changed from the first configuration to the second configuration, or vice versa, based on data from one or more of the electromagnetic energy sensors; and a data control unit. In an example, this eyewear can further comprise a forward-facing camera which receives light rays from a forward direction and a backward-facing camera which receives light rays from a backward direction.

In an example, in a first configuration an image-display member can display light rays from a forward direction received by the forward-facing camera. This enables the person to see ahead. In the second configuration, the image-display member can display light rays from a backward direction received by the backward-facing camera. This enables the person to see behind them. In an example, the person can toggle back and forth from seeing ahead vs. seeing behind by changing their brainwave patterns. In an example, the forward view can be maintained all the time and the rear view (which is toggled on or off) can take up only a small portion of the person's field of view.

In an example, a person can rotate the polar coordinate of the focal direction an image displayed by an image-display member by changing the power of their brainwaves in the Delta frequency band. In an example, a person can rotate the polar coordinate of the focal direction an image displayed by an image-display member by changing the power of their brainwaves in the Theta band. In an example, a person can rotate the polar coordinate of the focal direction an image displayed by an image-display member by changing the power of their brainwaves in the Alpha frequency band. In an example, a person can rotate the polar coordinate of the focal direction an image displayed by an image-display member by changing the power of their brainwaves in the Beta frequency band. In an example, a person can rotate the polar coordinate of the focal direction an image displayed by an image-display member by changing the power of their brainwaves in the Gamma band.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays images of the environment with a first scope or breadth and a second configuration which displays images of the environment with a first scope or breadth, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear with a variable focal distance, wherein this focal distance can be modified by a change in a person's electromagnetic brain activity. In an example, this device can enable a person to focus on environmental objects at difference distances by changing their brainwave pattern. In an example, a device can comprise virtual bifocal eyewear wherein this eyewear displays a distant object or a nearby object, depending on a person's brainwave pattern. In an example, a device can comprise virtual binoculars which display a normal-distance object or a far-distance object, depending on a person's brainwave pattern.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays environmental objects at a first focal distance and a second configuration which displays environmental objects at a second focal distance, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more image-display members within three inches of the person's head, wherein one or more of the image-display members has a first configuration which displays environmental objects at a first focal distance and a second configuration which displays environmental objects at a second focal distance, and wherein one or more image-display members are changed from the first configuration to the second configuration, or vice versa, based on data from one or more of the electromagnetic energy sensors; and a data control unit. In an example, the eyewear can further comprise a camera.

In an example, in a first configuration an image-display member can display a view of the environment which focuses on nearby object. In a second configuration, the image-display member can display a view of the environment which focuses on a far-away object.

In an example, a person can change the focal distance of an image displayed by an image-display member by changing the power of their brainwaves in the Delta frequency band. In an example, a person can change the focal distance of an image displayed by an image-display member by changing the power of their brainwaves in the Theta band. In an example, a person can change the focal distance of an image displayed by an image-display member by changing the power of their brainwaves in the Alpha frequency band. In an example, a person can change the focal distance of an image displayed by an image-display member by changing the power of their brainwaves in the Beta frequency band. In an example, a person can change the focal distance of an image displayed by an image-display member by changing the power of their brainwaves in the Gamma band.

In an example, eyewear embodied by this device can comprise a single near-eye display screen or other image-display member which spans (a portion of) the field of vision of one or a person's eyes. In an example, eyewear embodied by this device can comprise two near-eye display screens or other image-display members which each span (a portion of) the field of vision of one of a person's eyes and which together span (portions of) the fields of vision of both of the person's eyes. In an example, the contents displayed by two display screens or other image-display members to different eyes can differ in perspective so as to create the perception of a 3D image. In an example, eyewear can enable a person to change their image perception from 2D to 3D by changing their electromagnetic brain activity.

In an example, a person can change their perception of images from 2D to 3D, or vice versa, by changing the power of their brainwaves in the Delta frequency band. In an example, a person can change their perception of images from 2D to 3D, or vice versa, by changing the power of their brainwaves in the Theta band. In an example, a person can change their perception of images from 2D to 3D, or vice versa, by changing the power of their brainwaves in the Alpha frequency band. In an example, a person can change their perception of images from 2D to 3D, or vice versa, by changing the power of their brainwaves in the Beta frequency band. In an example, a person can change their perception of images from 2D to 3D, or vice versa, by changing the power of their brainwaves in the Gamma band.

In an example, a device can be embodied in eyewear which filters, blocks, amplifies, shifts, and/or analyzes light in one or more portions of the light spectrum. This modification of light transmission can be controlled by changes in a person's electromagnetic brain activity. In an example, a person can automatically filter out the display of light energy in a selected portion of the spectrum by changing their brainwave pattern. In an example, a person can shift the display of light in a portion of the light spectrum based on changes in their brainwave pattern. In an example, a person can extend their view into lower or upper non-visible ranges of the light spectrum, by shifting light upward or downward, based on changes in their brainwave activity. In an example, a person can adjust eyewear by changing their brainwave activity so that infrared light energy becomes displayed to them in a visible range. In an example, a person can adjust eyewear by changing their brainwave activity so that ultraviolet light energy becomes displayed to them in a visible range. In an example, a person can activate spectroscopic analysis of light energy, such as by using Fourier Transform, by changing their electromagnetic brain activity. In an example, a person can use eyewear to initiate spectral analysis of material composition, based on a change in their electromagnetic brain activity.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which does not display light in a selected range of the light spectrum and a second configuration which does display light in a selected range of the light spectrum, wherein the first portion can be zero, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which shifts the wavelength of light displayed by a first amount and a second configuration which shifts the wavelength of light displayed by a second amount, wherein the first amount can be zero, and wherein one or more image-display members are changed from the first configuration to the second configuration, or vice versa, based on data from one or more of the electromagnetic energy sensors; and (c) a data control unit that further comprises a power source and a data processor.

In an example, a person can shift the light spectrum of an image displayed by an image-display member in order to perceive normally-invisible light energy by changing the power of their brainwaves in the Delta frequency band. In an example, a person can shift the light spectrum of an image displayed by an image-display member in order to perceive normally-invisible light energy by changing the power of their brainwaves in the Theta band. In an example, a person can shift the light spectrum of an image displayed by an image-display member in order to perceive normally-invisible light energy by changing the power of their brainwaves in the Alpha frequency band. In an example, a person can shift the light spectrum of an image displayed by an image-display member in order to perceive normally-invisible light energy by changing the power of their brainwaves in the Beta frequency band. In an example, a person can shift the light spectrum of an image displayed by an image-display member in order to perceive normally-invisible light energy by changing the power of their brainwaves in the Gamma band.

In an example, an image-display member that is part of the eyewear disclosed herein can: display environmental objects; display virtual objects; or display a mixture of environmental and virtual objects. In an example, a person can modify the mixture of environmental and virtual objects displayed by changing their electromagnetic brain activity pattern. In an example, an image-display member that is part of the eyewear disclosed herein can: display a first view of environmental objects; display a second view of environmental objects; or display a mixture of the first and second views. In an example, a person can modify the mixture of the first and second views by changing their electromagnetic brain activity pattern. In an example, an image-display member that is part of the eyewear disclosed herein can: display a first type of virtual content; display a second type of virtual content; or display a mixture of the first and second types. In an example, a person can modify the mixture of the first and second types by changing their electromagnetic brain activity pattern.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays objects in the environment and has a second configuration that displays virtual objects, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more image-display members within three inches of the person's head, wherein one or more of the image-display members has a first configuration which displays objects in the environment and has a second configuration that displays virtual objects, and wherein one or more image-display members are changed from the first configuration to the second configuration, or vice versa, based on data from one or more of the electromagnetic energy sensors; and a data control unit. In an example, the eyewear can further comprise a camera. In an first configuration, an image-display member can display a view of the environment. In a second configuration, the image-display member can display a view of a virtual object or content.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays objects in the environment and has a second configuration that displays virtual objects superimposed on objects in the environment, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more image-display members within three inches of the person's head, wherein one or more of the image-display members has a first configuration which displays objects in the environment and has a second configuration that displays virtual objects superimposed on objects in the environment, and wherein one or more image-display members are changed from the first configuration to the second configuration, or vice versa, based on data from one or more of the electromagnetic energy sensors; and a data control unit. In an example, the eyewear can further comprise a camera. In a first configuration, an image-display member can display a view of just the environment. In a second configuration, the image-display member can display a view of a virtual object or content which is superimposed on a view of the environment.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays objects in the environment from a first view direction and has a second configuration that displays objects in the environment from a second view direction, and wherein one or more image-display members are changed from the first configuration to the second configuration, or vice versa, based on data from one or more of the electromagnetic energy sensors; and (c) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the person's head, wherein one or more of the image-display members has a first configuration which displays a first type of virtual content and has a second configuration that displays a second type of virtual content, and wherein one or more image-display members are changed from the first configuration to the second configuration, or vice versa, based on data from one or more of the electromagnetic energy sensors; and (c) a data control unit that further comprises a power source and a data processor.

In an example, a device can comprise an wearable eyewear frame with a specific configuration. In an example, a wearable frame can hold electromagnetic sensors, image-display members, and a data control unit in a selected configuration. In an example, a wearable frame can position image-display members in front of a person's eyes similar to the way in which a conventional pair of eyeglasses positions one or more lenses in front of a person's eyes. In an example, a wearable frame can be adjustable to enable adjustment of the configuration of electromagnetic sensors, image-display members, and/or a data control unit. In an example, adjustment can be manual. In an example, adjustment can be done by one or more actuators. In an example, adjustment can be automated.

In an example, a wearable eyewear frame can hold one or more electromagnetic sensors in contact with the surface of a person's head. In an example, one or more electromagnetic sensors can be spring-loaded to maintain compressive contact with the person's head. In an example, the degree of compressive contact can be adjusted. In an example, adjustment can be manual. In an example, adjustment can be automated to ensure continuous proper contact between the sensors and the surface of the person's head. In an example, a wearable frame can hold one or more electromagnetic sensors in contact with one or both sides of a person's head. In an example, a wearable frame can hold one or more electromagnetic sensors in contact in one or more locations between a person's ear and eye. In an example, a wearable frame can hold one or more electromagnetic sensors in contact with a person's forehead. In an example, a wearable frame can be similar to the frame of conventional eyeglasses, except that it has a central upward bulge, loop, or projection which holds an electrode in contact with a person's forehead.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is configured to span from the person's left ear to the person's face, then span across the front of the person's face including a portion of the person's nose, and then span from the person's face to the person's right ear.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is configured to span from the person's left ear to the person's face, then span across the front of the person's face including a portion of the person's forehead, and then span from the person's face to the person's right ear.

In an example, a device can include a wearable eyewear frame that is similar to a conventional eyeglasses frame, with the addition of an arcuate member that curves from one ear to the other, spanning the person's forehead. In an example, a device can comprise a wearable frame with a left-side frame piece between the left ear and left eye, a right-side frame piece between the right ear and right eye, and an arcuate member that curves from the left-side frame piece to the right-side frame piece spanning the person's forehead. In an example, an arcuate member can hold an electromagnetic sensor in contact with the person's forehead.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame has a first member which is configured to span from one of the person's ears to the person's nose and a second member which is configured to span from one of the person's ears to the person's forehead.

In an example, a device can be embodied in eyewear with a wearable frame which is similar to the frame of conventional eyeglasses, except that it includes an arcuate member or extension which loops completely around the rear of the person's head. In an example, such a rearward-looping arcuate member or extension can hold additional electromagnetic sensors against the surface of the person's head in order to improve the scope and accuracy of brain activity measurement. This can be particularly useful for measuring activity from the person's occipital lobe or cerebellum. Further, this rearward-looping arcuate member or extension can also help to hold the device on the person's head if the device is heavier than conventional eyeglasses.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is configured to span from the person's left ear to the person's face, then span across the front of the person's face, then span from the person's face to the person's right ear, and then span from the person's right ear to the person's left ear by looping around the rear of the person's head.

In an example, a device can be embodied in eyewear with a wearable frame which is similar to the frame of conventional eyeglasses, except that it includes an arcuate member or extension which loops completely over the top of the person's head. In an example, this upward-looping arcuate member or extension can hold additional electromagnetic sensors against the surface of the person's head in order to improve the scope and accuracy of brain activity measurement. This can be particularly useful for measuring activity from a person's parietal lobe and/or occipital lobe. Further, this upward-looping arcuate member or extension can also help to hold the device on a person's head if the device is heavier than conventional eyeglasses.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is configured to span from the person's left ear to the person's face, then span across the front of the person's face, then span from the person's face to the person's right ear, and then span from the person's right ear to the person's left ear by looping over the top of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame has a first portion which is configured to span from the person's left ear to the person's face, has a second portion which is configured to span across the front of the person's face, has a third portion which is configured to span from the person's face to the person's right ear, has a fourth portion which is configured to span from the person's right ear to the person's left ear by looping around the rear of the person's head; and has a fifth portion which is configured to span from the person's right ear to the person's left ear by looping over the top of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head; wherein this wearable frame is configured to curve around the anterior perimeter of the person's left ear, then span from the person's left ear to the person's face, then span across the front of the person's face, then span from the person's face to the person's right ear, and then curve around the anterior perimeter of the person's right ear; and wherein one or more of the electromagnetic energy sensors is located along a portion of the wearable frame which curves around the anterior perimeter of an ear.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head; wherein this wearable frame is configured to curve around the posterior perimeter of the person's left ear, then span from the person's left ear to the person's face, then span across the front of the person's face, then span from the person's face to the person's right ear, and then curve around the posterior perimeter of the person's right ear; and wherein one or more of the electromagnetic energy sensors is located along a portion of the wearable frame which curves around the posterior perimeter of an ear.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is substantially circular or elliptical and wherein this wearable frame spans both the person's forehead and the rear of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is substantially circular or elliptical and wherein this wearable frame encircles the person's head at an anterior acute angle in the range of 0 to 45 degrees with respect to a horizontal plane when the person's head is upright.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is substantially sinusoidal and wherein this wearable frame spans both the person's forehead and the rear of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is substantially sinusoidal and wherein the central axis of sinusoidal undulations encircles the person's head at an anterior acute angle in the range of 0 to 45 degrees with respect to a horizontal plane when the person's head is upright.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more image-display members configured to be within three inches of the surface of the person's head; (c) a data control unit that further comprises a power source and a data processor; and (d) a wearable frame which holds the one or more electromagnetic energy sensors and the one or more image-display members in their respective configurations within three inches of the surface of the person's head, wherein this wearable frame is substantially the shape of an ellipse projected downward onto the curvature of the person's head and wherein this wearable frame spans both the person's forehead and the rear of the person's head.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head; (c) one or more image-display members configured to be within three inches of the surface of the person's head; and (d) a data control unit that further comprises a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more light-transmitting optical members configured to be within three inches of the surface of the person's head; (c) one or more image-display members configured to be within three inches of the surface of the person's head; (d) a data control unit that further comprises a power source and a data processor; and (e) a wearable frame which holds the one or more electromagnetic energy sensors, the one or more light-transmitting optical members, and the one or more image-display members in their respective configurations.

In an example, an electromagnetic energy sensor can be an electrode. In an example, a light-transmitting optical member can be a lens. In an example, an image-display member can be a near-eye display screen. In an example, a data control unit can comprise a power source or transducer and a data processor. In an example, a wearable frame can be similar to the frame of conventional eyeglasses except that it includes a central upward bulge, loop, or projection which holds at least one electromagnetic energy sensor in contact with the person's forehead.

In an example, electromagnetic energy that is measured by one or more electromagnetic energy sensors can be statistically analyzed in order to identify significant changes in a person's electromagnetic brain activity. These significant changes in brain activity can then be used to control changes in the transmission of light through one or more light-transmitting optical members, to control changes in the images displayed by the image-display members, or both. In various examples, one or more primary statistical methods can be used to identify specific patterns of electromagnetic brain activity and/or specific changes in electromagnetic brain activity. In an example, data from one or more electromagnetic sensors can be filtered to remove artifacts before the application of a primary statistical method.

In an example, a pattern or change in electromagnetic brain activity can be a one-time pattern. In an example, a pattern of electromagnetic brain activity can repeat over time in a rhythmic manner. In an example, a primary statistical method can analyze repeating electromagnetic patterns by analyzing the frequency of repetition, the frequency band or range of repetition, their recurring amplitude, their wave phase, and/or their waveform. In an example repeating patterns and/or waveforms can be analyzed using Fourier Transform methods.

In an example, this device can enable a person to control the absorption, reflection, and/or refraction of light by one or more light-transmitting optical members by changing their electromagnetic brain activity. In an example, this device can enable a person to control the opacity and/or reflectivity of one or more light-transmitting optical members by changing their electromagnetic brain activity. In an example, this device can enable a person to control their focal distance, view direction, and/or view scope by changing their electromagnetic brain activity.

In an example, this device can enable a person to control the polar coordinate (around the circumference of their head) of their field of vision by changing their electromagnetic brain activity. In an example, this device can enable a person to control the absorption, transmission, or shifting of one or more portions of the light spectrum by changing their electromagnetic brain activity. In an example, this device can enable a person to control the dimensionality of their view (such as shifting from a two-dimensional view to three-dimensional view) by changing their electromagnetic brain activity.

In an example, a device can be embodied in a human-to-computer eyewear interface, comprising: (a) a head-worn structure, wherein this structure is configured to: span from a person's left ear to their face; then span across their face within their field of vision, including spanning a portion of their forehead; and then span from their face to their right ear; (b) one or more electromagnetic energy sensors, wherein these sensors are configured to be within three inches of the surface of a person's head, wherein these sensors are configured to measure electromagnetic energy from the person's head, and wherein these sensors are held in place by the head-worn structure; (c) one or more light-transmitting and/or image-displaying members, wherein these members are configured to be within three inches of the surface of the person's head, wherein these members are configured to transmit and/or display light into one or both of the person's eyes, and wherein these members are held in place by the head-worn structure; (d) a power source; and (e) a wireless data transmitter.

In an example, the head-worn structure can be a rigid or semi-rigid eyewear frame. In an example, the head-worn structure can be made of metal or a polymer. In an example, the head-worn structure can be similar to the frame of a conventional pair of eyeglasses with the addition of a bulge, loop, or projection which covers a portion of the person's forehead and holds one or more electromagnetic energy sensors in contact with the person's forehead. In an example, the head-worn structure can be flexible, elastic, compliant, and/or soft. In an example, the head-worn structure can be made of fabric.

In an example, an electromagnetic energy sensor can be an electrode. In an example, an electromagnetic energy sensor can be an electroencephalogram (EEG) electrode. In an example, an electromagnetic energy sensor can be a dry electrode. In an example, an electromagnetic energy sensor can measure electromagnetic brain activity. In an example, an electromagnetic sensor can be within an inch of the surface of a person's head. In an example, an electromagnetic sensor can be in direct contact with the surface of a person's head.

In an example, a light-transmitting and/or image-displaying member can be a lens and/or a near-eye display surface. In an example, light-transmitting and/or image-displaying member can be comprised of one or more optical elements selected from the group consisting of: simple lens, concave lens, concentric lenses, convex lens, diverging lens, asymmetric lens, compound lens, fly's eye lens, Fresnel lens, light-transducing element, microlens array, microspheres, optoelectronic lens, parabolic lens, wedge-shaped lens, liquid crystal, liquid lens, Digital Micromirror Device (DMD), Digital Light Processor (DLP), Electromagnetically Induced Transparency (EIT) structure, Liquid Crystal Display (LCD), MEMS-based lens array, MEMS-based mirror array, birefringent material, carbon nanotube, light-guiding metamaterial structure, light-guiding metamaterial structure, light-guiding tube, metamaterial light channel, microscale glass beads, nanorods, nanoscale gratings, nanotubes, etched waveguide, nanoimprint lithography pathways, resonant grating filter, Split Ring Resonators (SRRs), thermoplastic nanoimprint pathways, crystal, crystal array, crystalline structures, photonic metamaterial, photonic crystal, fiber optics, optical fiber, polarizing filter, cylindrical prism, prism, wedge prism, acrylic mirror, concentric reflective surfaces, dielectric mirror, parabolic mirror, reflector array, and retroreflective structure.

In an example, a person can use this eyewear to wirelessly control the operation of a separate wearable device by changing their electromagnetic brain activity in the Delta, Theta, Alpha, Beta, and/or Gamma frequency bands. In an example, a person can use this eyewear to wirelessly control the operation of a wristwatch, smart watch, fitness watch, watch phone, bracelet phone, smart bracelet, fitness bracelet, smart wrist band, electronically-functional wrist band, other wrist-worn electronic device by changing their electromagnetic brain activity in the Delta, Theta, Alpha, Beta, and/or Gamma frequency bands.

In an example, a person can use this eyewear to wirelessly control the operation of a smart button, electronically-functional button, pin, brooch, pendant, beads, neck chain, necklace, dog tags, locket, or medallion by changing their electromagnetic brain activity in the Delta, Theta, Alpha, Beta, and/or Gamma frequency bands. In an example, a person can use this eyewear to wirelessly control the operation of a smart finger ring, electronically-functional finger ring, electronically-functional earring, nose ring, or ear bud or clip by changing their electromagnetic brain activity in the Delta, Theta, Alpha, Beta, and/or Gamma frequency bands. In an example, a person can use this eyewear to wirelessly control the operation of an article of smart clothing, an electronically-functional shirt, electronically-functional pants, or a smart belt by changing their electromagnetic brain activity in the Delta, Theta, Alpha, Beta, and/or Gamma frequency bands.

In an example, a person can use this eyewear to wirelessly control the operation of a separate mobile or handheld device by changing their electromagnetic brain activity in the Delta, Theta, Alpha, Beta, and/or Gamma frequency bands. In an example, a person can use this eyewear to wirelessly control the operation of a smart phone, mobile phone, holophone, or cellular phone by changing their electromagnetic brain activity in the Delta, Theta, Alpha, Beta, and/or Gamma frequency bands. In an example, a person can use this eyewear to wirelessly control the operation of an electronic tablet, electronic pad, and other electronically-functional handheld device by changing their electromagnetic brain activity in the Delta, Theta, Alpha, Beta, and/or Gamma frequency bands.

In an example, a person can use this eyewear to wirelessly control the operation of a relatively fixed-location electronically-functional device by changing their electromagnetic brain activity in the Delta, Theta, Alpha, Beta, and/or Gamma frequency bands. In an example, a person can use this eyewear to wirelessly control the operation of a laptop computer, desktop computer, or internet terminal by changing their electromagnetic brain activity in the Delta, Theta, Alpha, Beta, and/or Gamma frequency bands. In an example, a person can use this eyewear to wirelessly control the operation of a smart appliance or home control system by changing their electromagnetic brain activity in the Delta, Theta, Alpha, Beta, and/or Gamma frequency bands. In an example, a device can comprise brainwave-monitoring eyewear which is part of the Internet of Thinks (IOT).

In an example, a device can be embodied in eyewear with one or more electromagnetic energy sensors which enables a person to move a cursor on a computer display screen by changing the power of their brainwaves in the Delta frequency band. In an example, a device can be embodied in eyewear with one or more electromagnetic energy sensors which enables a person to move a cursor on a computer display screen by changing the power of their brainwaves in the Theta frequency band. In an example, a device can be embodied in eyewear with one or more electromagnetic energy sensors which enables a person to move a cursor on a computer display screen by changing the power of their brainwaves in the Alpha frequency band. In an example, a device can be embodied in eyewear with one or more electromagnetic energy sensors which enables a person to move a cursor on a computer display screen by increasing or decreasing the power of their brainwaves in the Beta frequency band.

In an example, a device can be embodied in eyewear with one or more electromagnetic energy sensors which enables a person to select an option in a computer menu interface by changing the power of their brainwaves in the Delta frequency band. In an example, a device can be embodied in eyewear with one or more electromagnetic energy sensors which enables a person to select an option in a computer menu interface by changing the power of their brainwaves in the Theta frequency band. In an example, a device can be embodied in eyewear with one or more electromagnetic energy sensors which enables a person to select an option in a computer menu interface by changing the power of their brainwaves in the Alpha frequency band. In an example, a device can be embodied in eyewear with one or more electromagnetic energy sensors which enables a person to select an option in a computer menu interface by increasing or decreasing the power of their brainwaves in the Beta frequency band.

In an example, a device can be embodied in a human-to-computer interface system, comprising: (a) eyewear which monitors a person's electromagnetic brain activity, wherein this eyewear further comprises: a head-worn structure which is configured to span from a person's left ear to their face, across their face within their field of vision including a portion of their forehead, and from their face to their right ear; one or more electromagnetic energy sensors which are configured to be within three inches of the surface of a person's head, which measure electromagnetic energy from the person's head, and which are held in place by the head-worn structure; a power source; and a wireless data transmitter; and (b) a separate wearable, mobile, or fixed-location electronic device with which the eyewear is in wireless communication, wherein the operation of this separate device is controlled changes in the person's electromagnetic brain activity based on data from the eyewear.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; and (c) a data control unit that further comprises a power source and a data processor. In an example, this eyewear can further comprise one or more image-display members.

In an example, a device can be embodied in eyewear that modifies a person's visual perception and/or imaging based on electromagnetic energy measured from the person's head, comprising: one or more electromagnetic energy sensors within three inches of the surface of a person's head and configured to measure electromagnetic energy from the person's head; one or more wearable image-recording members; and a data control unit. In an example, an image-recording member can be a camera. In an example, a data control unit can further comprise a power source and a data processor.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors, the one or more light-transmitting optical members, and the one or more image-recording members in their respective configurations.

In an example, this eyewear can further comprise one or more image-display members. In an example, an electromagnetic energy sensor can be an electrode. In an example, a wearable frame can be similar to the frame of conventional eyeglasses except that it includes a central upward bulge, loop, or projection which holds at least one electromagnetic energy sensor in contact with a person's forehead.

In an example, a data control unit can comprise a power source or transducer and a data processor. In an example, a data control unit can be in direct electrical communication with one or more electromagnetic energy sensors by wires or other electrically-conductive pathways. In an example, a data control unit can be in wireless communication with one or more electromagnetic energy sensors. In an example, a data control unit can be in direct electrical communication with one or more wearable image-recording members by wires or other electrically-conductive pathways. In an example, a data control unit can be in wireless communication with one or more wearable image-recording members.

In an example, an image-display member can be a near-eye display screen. In an example, an image-recording member can be a wearable camera. In an example, an image-recording member can be selected from the group consisting of: 35 mm camera, analog or film camera, camcorder, CCD camera, CMOS camera, digital camera, motion picture camera, SLR camera, and video camera. In an example, an image-recording member can be integrated into an eyewear frame.

In an example, electromagnetic energy that is measured by one or more electromagnetic energy sensors can be statistically analyzed in order to identify significant changes in a person's electromagnetic brain activity. These significant changes in brain activity can then be used to control changes in the transmission of light through one or more light-transmitting optical members, to control changes in the images recorded by an image-recording member, or both. In various examples, one or more primary statistical methods can be used to identify specific patterns of electromagnetic brain activity and/or specific changes in electromagnetic brain activity. In an example, data from one or more electromagnetic sensors can be filtered to remove artifacts before the application of a primary statistical method.

In an example, this device can enable a person to control the focal distance, view direction, and/or view scope of an image-recording member by changing their electromagnetic brain activity. In an example, this can be done by changing the configuration of an image-recording optical member. In an example, this can be done by changing a view from that of a first image-recording optical member to that of a second image-recording optical member. In an example, this device can enable a person to see what is behind them by changing their electromagnetic activity. In an example, first image-recording member can point toward objects in front of a person and a second image-recording member can point toward objects behind the person. In an example, such eyewear can be especially useful for grade school teachers.

In an example, this device can enable a person to control the absorption, transmission, or shifting of one or more portions of the light spectrum recorded by an image-recording member by changing their electromagnetic brain activity. In an example, this device can enable a person to control the dimensionality of their view (such as shifting from a two-dimensional view to three-dimensional view) by changing their electromagnetic brain activity.

In an example, this device can enable a person to change the length of time that a recorded image is kept in memory by changing their electromagnetic brain activity. In an example, an image-recording member can record video images constantly, but the images can be erased after a selected period time unless their erasure is cancelled by a selected pattern of electromagnetic brain activity. In an example, this selected pattern of electromagnetic brain activity can be voluntary and conscious. In an example, this selected pattern of electromagnetic brain activity can be involuntary and/or unconscious.

In an example, this device can enable a person to start video recording by changing their electromagnetic brain activity. In an example, an image-recording member can start recording video images based on a selected pattern of electromagnetic brain activity. In an example, this selected pattern of electromagnetic brain activity can be voluntary and conscious. In an example, this selected pattern of electromagnetic brain activity can be involuntary and/or unconscious. In an example, involuntary initiation of video recording based on a selected pattern of brain activity can serve a safety or emergency purpose.

In an example, this device can enable a person to start or stop the wireless transmission of video images by changing their electromagnetic brain activity. In an example, an image-recording member can start or stop recording video images based on a selected pattern of electromagnetic brain activity. In an example, this selected pattern of electromagnetic brain activity can be a voluntary and conscious one. In an example, this selected pattern of electromagnetic brain activity can be an involuntary and/or unconscious one. In an example, involuntary initiation of video recording based on a selected pattern of brain activity can serve a safety or emergency purpose.

In an example, a device can further comprise a specific shape of wearable frame. In an example, a wearable frame can hold electromagnetic sensors, an image-recording member, and a data control unit in a selected configuration. In an example, a wearable frame can hold one or more electromagnetic sensors in contact with the surface of a person's head. In an example, a wearable frame can hold one or more electromagnetic sensors in contact with one or both sides of a person's head. In an example, a wearable frame can hold one or more electromagnetic sensors in contact in one or more locations between a person's ear and eye. In an example, a wearable frame can hold one or more electromagnetic sensors in contact with a person's forehead. In an example, a wearable frame can be similar to the frame of conventional eyeglasses, except that it has a central upward bulge, loop, or projection which holds an electrode in contact with a person's forehead.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members in their respective configurations. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is configured to span from the person's left ear to the person's face, then span across the front of the person's face including the person's nose, and then span from the person's face to the person's right ear. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; (b) one or more wearable image-recording members; (c) a data control unit that further comprises a power source and a data processor; and (d) a wearable frame which holds the one or more electromagnetic energy sensors and the one or more image-recording members, wherein this wearable frame is configured to span from the person's left ear to the person's face, then span across the front of the person's face including the person's forehead, and then span from the person's face to the person's right ear. In an example, this eyewear can further comprise one or more image-display members.

In an example, a device can include a wearable eyewear frame that is similar to a conventional eyeglasses frame, with the addition of an arcuate member that curves from one ear to the other, spanning the person's forehead. In an example, a device can comprise a wearable frame with a left-side frame piece between the left ear and left eye, a right-side frame piece between the right ear and right eye, and an arcuate member that curves from the left-side frame piece to the right-side frame piece including spanning the person's forehead. In an example, an arcuate member such as one of these above-described can hold an electromagnetic sensor in contact with the person's forehead.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head, wherein at least one of the electromagnetic energy sensors is configured to measure electromagnetic energy from the person's forehead; (b) one or more wearable image-recording members; (c) a data control unit that further comprises a power source and a data processor; and (d) a wearable frame which holds the one or more electromagnetic energy sensors and the one or more image-recording members, wherein this wearable frame has a first member which is configured to span from one of the person's ears to the person's nose and a second member which is configured to span from one of the person's ears to the person's forehead. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is configured to span from the person's left ear to the person's face, then span across the front of the person's face, then span from the person's face to the person's right ear, and then span from the person's right ear to the person's left ear by looping around the rear of the person's head. In an example, this eyewear can further comprise one or more image-display members.

In an example, a device can be embodied in eyewear with a wearable frame which is similar to the frame of conventional eyeglasses, except that it includes an arcuate member which loops completely over the top of the person's head. In an example, this upward-looping arcuate member can hold additional electromagnetic sensors against the surface of the person's head in order to improve the scope and accuracy of brain activity measurement. This can be particularly useful for measuring activity from the person's parietal lobe and/or occipital lobe. Further, this upward-looping arcuate member can also help to hold the device on the person's head if the device is heavier than conventional eyeglasses.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is configured to span from the person's left ear to the person's face, then span across the front of the person's face, then span from the person's face to the person's right ear, and then span from the person's right ear to the person's left ear by looping over the top of the person's head. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame has a first portion which is configured to span from the person's left ear to the person's face, has a second portion which is configured to span across the front of the person's face, has a third portion which is configured to span from the person's face to the person's right ear, has a fourth portion which is configured to span from the person's right ear to the person's left ear by looping around the rear of the person's head; and has a fifth portion which is configured to span from the person's right ear to the person's left ear by looping over the top of the person's head. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members; wherein this wearable frame is configured to curve around the anterior perimeter of the person's left ear, then span from the person's left ear to the person's face, then span across the front of the person's face, then span from the person's face to the person's right ear, and then curve around the anterior perimeter of the person's right ear; and wherein one or more of the electromagnetic energy sensors is located along a portion of the wearable frame which curves around the anterior perimeter of an ear. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members; wherein this wearable frame is configured to curve around the posterior perimeter of the person's left ear, then span from the person's left ear to the person's face, then span across the front of the person's face, then span from the person's face to the person's right ear, and then curve around the posterior perimeter of the person's right ear; and wherein one or more of the electromagnetic energy sensors is located along a portion of the wearable frame which curves around the posterior perimeter of an ear. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is substantially circular or elliptical and wherein this wearable frame spans both the person's forehead and the rear of the person's head. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is substantially circular or elliptical and wherein this wearable frame encircles the person's head at an anterior acute angle in the range of 0 to 45 degrees with respect to a horizontal plane when the person's head is upright. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is substantially sinusoidal and wherein this wearable frame spans both the person's forehead and the rear of the person's head. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is substantially sinusoidal and wherein the central axis of sinusoidal undulations encircles the person's head at an anterior acute angle in the range of 0 to 45 degrees with respect to a horizontal plane when the person's head is upright. In an example, this eyewear can further comprise one or more image-display members.

In an example, a device can be embodied in eyewear that modifies a person's visual perception and/or imaging based on electromagnetic energy measured from the person's head, comprising: (a) one or more electromagnetic energy sensors configured to be within three inches of the surface of a person's head and configured to measure electromagnetic energy from the person's head; (b) one or more wearable image-recording members; (c) a data control unit that further comprises a power source and a data processor; and (d) a wearable frame which holds the one or more electromagnetic energy sensors and the one or more image-recording members, wherein this wearable frame is substantially the shape of an ellipse projected downward onto the curvature of the person's head and wherein this wearable frame spans both the person's forehead and the rear of the person's head. In an example, this eyewear can further comprise one or more image-display members.

In an example, a shape-transforming eyewear device for collecting data concerning electromagnetic brain activity can comprise: (a) a face-spanning support member which is configured to span an upper portion of a person's face; (b) at least one optical member which transmits light from the person's environment and/or light from a virtual image display to at least one of the person's eyes; (c) an electromagnetic energy sensor, wherein this electromagnetic energy sensor has a first sensor configuration wherein it is configured to be at a selected location relative to the surface of the person's head in order to collect data concerning electromagnetic brain activity and wherein this electromagnetic energy sensor has a second sensor configuration wherein it is not at this selected location; and (d) a movable sensor arm which holds the electromagnetic energy sensor and is at least partially connected to the face-spanning support member, wherein this moveable sensor arm has a first arm configuration which holds the electromagnetic energy sensor in the first sensor configuration, wherein this moveable sensor arm has a second arm configuration which holds the electromagnetic energy sensor in the second sensor configuration, and wherein this moveable sensor arm is moved relative to the face-spanning support member in order to change from the first arm configuration to the second arm configuration, or vice versa.

In an example, a shape-changing eyewear device for measuring electromagnetic brain activity can comprise: (a) a face-spanning support member which is configured to span the upper portion of a person's face; (b) at least one optical member which is configured to transmit light from the person's environment, from a virtual image display, or from both sources to at least one of the person's eyes; (c) an electromagnetic energy sensor which has a first sensor configuration wherein it is configured to be in contact with the surface of the person's head at a selected location in order to measure electromagnetic brain activity and which has a second sensor configuration wherein it is not at this selected location; and (d) a movable sensor arm which is at least partially connected to the face-spanning support member, wherein this moveable sensor arm is moved relative to the face-spanning support member from a first arm configuration to a second arm configuration while remaining at least partially connected to the face-spanning support member, wherein this moveable sensor arm holds the electromagnetic energy sensor in the first sensor configuration when the moveable sensor arm is in the first arm configuration, and wherein this moveable sensor arm holds the electromagnetic energy sensor in the second sensor configuration when the moveable sensor arm is in the second arm configuration.

In an example, a face-spanning support member can horizontally span the upper half of a person's face. In an example, a face-spanning support member can span a person's eyes, eyebrows, and/or forehead. In an example, a face-spanning support member can span from one ear to the other ear. In an example, a face-spanning support member can span from one side of a person's head to the other side. In an example, a face-spanning support member can be symmetric with respect to the central longitudinal (right-vs.-left) cross-sectional plane of a person's head. In another example, a face-spanning support member can be asymmetric with respect to this plane.

In an example, a face-spanning support member can have: two side portions, each of which are configured to span from an ear to the front of the face; and a front portion which spans the front of the person's face from one side to the other. In an example, these three portions can be three connected pieces. In an example, these three portions can be connected by hinge mechanisms. In an example, these three portions can be one continuous piece.

In an example, a side portion of a face-spanning support member can be in substantially horizontal alignment with a person's eyes (when the person is standing up). In an example, a side portion can be in substantially horizontal alignment with a person's eyebrows. In an example, a side portion can be in substantially horizontal alignment with a person's forehead. In an example, a rear part of a side portion can be horizontally-aligned with the top of a person's ears and a front part of this side portion can be horizontally-aligned with the person's eyebrows. In an example, a side portion can have a relatively narrow and constant cross-sectional size. In an example, a side portion can flare from a rear part to a front part. In an example, a side portion can be substantially straight. In an example, a side portion can be arcuate.

In an example, a front portion of a face-spanning support member can be in substantially horizontal alignment with a person's eyes (when the person is standing up). In an example, a front portion can be in substantially horizontal alignment with a person's eyebrows. In an example, a front portion can be in substantially horizontal alignment with a person's forehead. In an example, a front portion can be substantially straight. In an example, a front portion can be arcuate. In an example, a front portion can have a central upward curve and/or other protrusion which spans upward onto the middle portion of a person's forehead. In an example, a front portion can have two upward curves and/or other protrusions which span upwards onto the right and left portions of a person's forehead.

In an example, a face-spanning support member can be arcuate. In an example, a face-spanning support member can wrap around a portion of a person's head, from one side to another, and span a portion of the person's upper face in the process. In an example, a face-spanning support member can span a portion of the circumference of a person's head, spanning a portion of the person's face in the process. In an example, a face-spanning support member can span the entire circumference of a person's head, spanning a portion of the person's face in the process. In an example, a face-spanning support member can have a shape which is selected from the group consisting of: spline and/or series of adjacent straight lines; conic section; circle, semicircle, or other section of a circle; ellipse or a section of an ellipse; and sinusoidal shape

In an example, a face-spanning support member can comprise multiple connected pieces. In an example, one or more of these individual pieces can be substantially straight. In an example, a face-spanning support member can have a shape comprised of a series of substantially-straight pieces which are connected to each other. In an example, these connections can be hinges. In an example, one or more of these individual pieces can be arcuate. In an example, a face-spanning support member can comprise three connected pieces: two side pieces and one front piece. In an example, the front piece can hold two optical members, such as lenses. In an example, these three pieces can be connected by hinges. In an example, a face-spanning support member can comprise eyeglass frames.

In an example, a face-spanning support member can span a portion of the circumference of a person's head from one ear to the other ear. In an example, a face-spanning support member can span a portion of the circumference of a person's head from one side to the other side. In an example, the rear portions of a face-spanning support member can rest on top of a person's ears. In an example, the read portions of a face-spanning support member can curve around the rear portions of a person's ears.

In an example, a face-spanning support member can span the entire circumference of a person's head from front to back. In an example, a face-spanning support member can span the entire circumference of a person's head in a substantially horizontal manner. In an example, a face-spanning support member can span the entire circumference of a person's head in a plane which forms an angle with the horizontal plane (when the person is standing up) which is less than 50 degrees. In an example, the middle portion of a face-spanning support member which spans the entire circumference of a person's head can rest on top of the person's ears.

In an example, a face-spanning support member can have a longitudinal axis as it spans a portion of a person's head. In an example, this longitudinal axis can be arcuate. In an example, this longitudinal axis can have a spline shape. In an example, a face-spanning support member can have lateral cross-sectional areas which are perpendicular to this longitudinal axis. In an example, the heights of these lateral cross-sectional areas can be less than two inches (with the exception of a section which encompasses the perimeter of a lens). In an example, the heights of these lateral cross-sectional areas can be substantially constant along the side portion of a face-spanning support member which spans from a person's ear to the front of their face. In an example, the heights of lateral cross-sectional areas can vary along the side portion of a face-spanning support member which spans from a person's ear to the front of their face.

In an example, a face-spanning support member can be made of metal, a polymer, a textile, or a combination thereof. In an example, a face-spanning support member can be substantially rigid. In an example, a face-spanning support member can be flexible. In an example, a face-spanning support member can be sufficiently flexible to be placed around (a portion of) a person's head but also sufficiently resilient to be held against a person's head by tension once it is placed around (a portion of) a person's head. In an example, a face-spanning support member can be elastic. In an example, a face-spanning support member can be sufficiently elastic so that it can be placed around (a portion of) a person's head, but also sufficiently resilient to be held against a person's head by tension once it is placed around (a portion) of a person's head.

In an example, a face-spanning support member can be fastened around (a portion) of a person's head by one or more attachment mechanisms selected from the group consisting of: band, elastic, loop, strap, chain, clip, clasp, snap, buckle, clamp, button, hook, pin, plug, hook-and-eye mechanism, adhesive, tape, electronic and/or electromagnetic connector, electronic plug, magnetic connector, threaded member, fiber, thread, and zipper.

In an example, an optical member can transmit, channel, and/or guide light from a person's environment into one or both of the person's eyes. In an example, an optical member can be a lens. In an example, an optical member can be a convex or concave lens. In an example, this device can comprise a single optical member. In an example, this device can comprise a single lens. In an example, this device can comprise two optical members. In an example, this device can comprise two lenses. In an example, two optical members can comprise eyeglass lenses. In an example, a face-spanning support member and two optical members can together comprise a pair of eyeglasses. In an example, a face-spanning support member and two optical members can together comprise a pair of sunglasses. In an example, this device can comprise electronically-functional eyeglasses, electronically-functional goggles, an electronically-functional visor, or other electronically-functional eyewear.

In an example, an optical member can be selected from the group consisting of: simple lens, concave lens, convex lens, bifocal lens, trifocal lens, asymmetric lens, microlens array, MEMS-based lens array, optoelectronic lens, parabolic lens, wedge-shaped lens, liquid lens, Digital Micromirror Device (DMD), Digital Light Processor (DLP), Electromagnetically Induced Transparency (EIT) structure, MEMS-based mirror array, birefringent material, carbon nanotube, light-guiding metamaterial structure, light-guiding tubes, metamaterial light channel, nanorods, nanoscale gratings, nanotubes, etched waveguide, nanoimprint lithography pathways, resonant grating filter, Split Ring Resonator (SRR), thermoplastic nanoimprint pathways, crystalline structures, photonic metamaterial, photonic crystal, optical fiber, polarizing filter, prism, wedge prism, dielectric mirror, parabolic mirror, other mirror, reflector array, and retroreflective structure.

In an example, an optical member comprise a virtual image display, computer display, and/or electronic screen which emits, transmits, channels, and/or guides light into one or both of a person's eyes. In an example, an optical member can display an image in a person's field of vision. In an example, an optical member can display one or more virtual objects in a person's field of vision. In an example, an optical member can display virtual objects in juxtaposition with physical objects in a person's field of vision. In an example, an optical member can display information concerning physical objects in a person's field of vision. In an example, this device can comprise the visual component of a virtual reality and/or augmented reality system.

In an example, an optical member can be selected from the group consisting of: virtual image display, computer screen, heads up display, array or matrix of light-emitting members, infrared display, laser display, light emitting diodes (LED), array or matrix of light emitting diodes (LEDs), waveguide, array or matrix of fiber optic members, optoelectronic lens, computer display, camera or other imaging device, light-emitting member array or matrix, light display array or matrix, liquid crystal display (LCD), and image projector.

In an example, an electromagnetic energy sensor can collect data concerning electromagnetic brain activity from a selected location in a first sensor configuration, but not in a second sensor configuration. In an example, a device can comprise shape-transforming eyewear which transitions from the first sensor configuration to the second sensor configuration, and vice versa. In an example, an electromagnetic energy sensor can be in direct contact with the surface of a person's head at a selected location in a first sensor configuration and not be in direct contact with the surface of the person's head at this selected location in a second sensor configuration. In an example, an electromagnetic energy sensor can be in direct contact with the surface of a person's head in a first sensor configuration and not in direct contact with the surface of the person's head in a second sensor configuration.

In an example, a selected location from which an electromagnetic energy sensor collects data concerning electromagnetic brain activity can be on (or near) the surface of the person's forehead and/or temple. In an example, the selected location from which an electromagnetic energy sensor collects data concerning electromagnetic brain activity can be on a person's ear and/or within the person's ear canal. In an example, the selected location from which an electromagnetic energy sensor collects data concerning electromagnetic brain activity can be selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. Also, a reference location can be selected from sites A1 and A2.

In an example, an electromagnetic energy sensor can be sufficiently close to the surface of a person's head so as to be in electromagnetic communication with body tissue. In an example, an electromagnetic energy sensor can be in electromagnetic communication with body tissue at a selected location in a first sensor configuration, but not in a second sensor configuration. In an example, a device can comprise shape-transforming eyewear which transitions from the first sensor configuration to the second sensor configuration, and vice versa.

In an example, an electromagnetic energy sensor can measure the conductivity, voltage, resistance, and/or impedance of electromagnetic energy transmitted through and/or emitted from a portion of a person's head. In an example, an electromagnetic energy sensor can be an electroencephalographic (EEG) sensor. In an example, an electromagnetic energy sensor can be a dry electrode. In an example, an electromagnetic energy sensor can collect data on electromagnetic energy patterns and/or electromagnetic fields which are naturally generated by electromagnetic brain activity. In an example, an electromagnetic energy sensor can be used in combination with an electromagnetic energy emitter. In an example, an electromagnetic energy emitter can be in contact with the surface of a person's head. In an example, an electromagnetic energy sensor can measure the conductivity, voltage, resistance, and/or impedance of electromagnetic energy emitted from an electromagnetic energy emitter and transmitted through a portion of a person's head.

In an example, this device can comprise a plurality of electromagnetic energy sensors which collect data concerning electromagnetic brain activity from different selected locations. In an example, an electromagnetic energy sensor can measure the conductivity, voltage, resistance, or impedance of electromagnetic energy that is transmitted between two locations. In an example, the locations for a plurality of electromagnetic energy sensors can be selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, a plurality of electromagnetic energy sensors can be located in a symmetric manner with respect to the central longitudinal right-vs.-left plane of a person's head. In an example, electromagnetic brain activity data from a selected recording location (relative to a reference location) is a channel. In an example, electromagnetic brain activity data from multiple recording places is a montage.

In an example, data from one or more electromagnetic energy sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter.

In an example, data from an electromagnetic energy sensor can be analyzed using Fourier transformation methods in order to identify repeating energy patterns in clinical frequency bands. In an example, these clinical frequency bands can be selected from the group consisting of: Delta, Theta, Alpha, Beta, and Gamma. In an example, the relative and combinatorial power levels of energy in two or more different clinical frequency bands can be analyzed. In an example, a person can receive real-time feedback based on analysis of data concerning their electromagnetic brain activity. In an example, a person can control a computer or other device by self-modifying their electromagnetic brain activity.

In an example, a primary statistical method can comprise finding the mean or average value of data from one or more brain activity channels during a period of time. In an example, a statistical method can comprise identifying a significant change in the mean or average value of data from one or more brain activity channels. In an example, a statistical method can comprise finding the median value of data from one or more brain activity channels during a period of time. In an example, a statistical method can comprise identifying a significant change in the median value of data from one or more brain activity channels. In an example, a statistical method can comprise identifying significant changes in the relative mean or median data values among multiple brain activity channels. In an example, a statistical method can comprise identifying significant changes in mean data values from a first set of electrode locations relative to mean data values from a second set of electrode locations. In an example, a statistical method can comprise identifying significant changes in mean data recorded from a first region of the brain relative to mean data recorded from a second region of the brain.

In an example, a primary statistical method can comprise finding the minimum or maximum value of data from one or more brain activity channels during a period of time. In an example, a statistical method can comprise identifying a significant change in the minimum or maximum value of data from one or more brain activity channels. In an example, a statistical method can comprise identifying significant changes in the relative minimum or maximum data values among multiple brain activity channels. In an example, a statistical method can comprise identifying significant changes in minimum or maximum data values from a first set of electrode locations relative to minimum or maximum data values from a second set of electrode locations. In an example, a statistical method can comprise identifying significant changes in minimum or maximum data values recorded from a first region of the brain relative to minimum or maximum data values recorded from a second region of the brain.

In an example, a primary statistical method can comprise finding the variance or the standard deviation of data from one or more brain activity channels during a period of time. In an example, a statistical method can comprise identifying a significant change in the variance or the standard deviation of data from one or more brain activity channels. In an example, a statistical method can comprise identifying significant changes in the covariation and/or correlation among data from multiple brain activity channels. In an example, a statistical method can comprise identifying significant changes in the covariation or correlation between data from a first set of electrode locations relative and data from a second set of electrode locations. In an example, a statistical method can comprise identifying significant changes in the covariation or correlation of data values recorded from a first region of the brain and a second region of the brain.

In an example, a primary statistical method can comprise finding the mean amplitude of waveform data from one or more channels during a period of time. In an example, a statistical method can comprise identifying a significant change in the mean amplitude of waveform data from one or more channels. In an example, a statistical method can comprise identifying significant changes in the relative means of wave amplitudes from one or more channels. In an example, a statistical method can comprise identifying significant changes in the amplitude of electromagnetic signals recorded from a first region of the brain relative to the amplitude of electromagnetic signals recorded from a second region of the brain.

In an example, a primary statistical method can comprise finding the power of waveform brain activity data from one or more channels during a period of time. In an example, a statistical method can comprise identifying a significant change in the power of waveform data from one or more channels. In an example, a statistical method can comprise identifying significant changes in the relative power levels of one or more channels. In an example, a statistical method can comprise identifying significant changes in the power of electromagnetic signals recorded from a first region of the brain relative to the power of electromagnetic signals recorded from a second region of the brain.

In an example, a primary statistical method can comprise finding a frequency or frequency band of waveform and/or rhythmic brain activity data from one or more channels which repeats over time. In an example, Fourier transformation methods can be used to find a frequency or frequency band of waveform and/or rhythmic data which repeats over time. In an example, a statistical method can comprise decomposing a complex waveform into a combination of simpler waveforms which each repeat at a different frequency or within a different frequency band. In an example, Fourier transformation methods can be used to decomposing a complex waveform into a combination of simpler waveforms which each repeat at a different frequency or within a different frequency band.

In an example, a primary statistical method can comprise identifying significant changes in the amplitude, power level, phase, frequency, and/or oscillation of waveform data from one or more channels. In an example, a primary statistical method can comprise identifying significant changes in the amplitude, power level, phase, frequency, and/or oscillation of waveform data within a selected frequency band. In an example, a primary statistical method can comprise identifying significant changes in the relative amplitudes, power levels, phases, frequencies, and/or oscillations of waveform data among different frequency bands. In various examples, these significant changes can be identified using Fourier transformation methods.

In an example, brainwaves (or other rhythmic, cyclical, and/or repeating electromagnetic signals associated with brain activity) can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier transformation methods. In an example, brainwaves can be measured and analyzed using five common clinical frequency bands: Delta, Theta, Alpha, Beta, and Gamma.

In an example, Delta brainwaves can be measured and analyzed within the frequency band of 1 to 4 Hz. In various examples, Delta brainwaves (or other rhythmic, cyclical, and/or repeating electromagnetic signals associated with brain activity) can be measured and analyzed within a frequency band selected from the group consisting of: 0.5-3.5 Hz, 0.5-4 Hz, 1-3 Hz, 1-4 Hz, and 2-4 Hz. In an example, Theta brainwaves can be measured and analyzed within the frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz.

In an example, Alpha brainwaves can be measured and analyzed within the frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, Beta brainwaves can be measured and analyzed within the frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, Gamma brainwaves can be measured and analyzed within the frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz.

In an example, data concerning electromagnetic brain activity which is collected by one or more electromagnetic energy sensors can be analyzed using one or more statistical methods selected from the group consisting of: multivariate linear regression or least squares estimation; factor analysis; Fourier transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; AI (e.g. machine learning); power spectral density; power spectrum analysis; probit model; time-series analysis; inter-band mean; inter-band ratio; inter-channel mean; inter-channel ratio; inter-montage mean; inter-montage ratio; multi-band covariance analysis; multi-channel covariance analysis; and analysis of wave frequency, wave frequency band, wave amplitude, wave phase, and wave form or morphology. In an example, wave form or morphology can be identified from the group consisting of: simple sinusoidal wave, composite sinusoidal wave, simple saw-tooth wave, composite saw-tooth wave, biphasic wave, tri-phasic wave, and spike.

In an example, a moveable sensor arm can be at least partially connected to a face-spanning support member and can hold at least one electromagnetic energy sensor which collects data concerning electromagnetic brain activity. In an example, a moveable sensor arm can be moved relative to a face-spanning support member from a first arm configuration to a second arm configuration, and vice versa. In an example, in the first arm configuration, the moveable sensor arm holds the electromagnetic energy sensor in a selected location from which the electromagnetic energy sensor collects data concerning electromagnetic brain activity. In the second arm configuration the moveable sensor arm holds the electromagnetic energy sensor in a different location.

In an example, a moveable sensor arm can be visually less-obtrusive in a second arm configuration than in a first arm configuration. This allows this eyewear device to collect data concerning electromagnetic brain activity in a more-obtrusive first configuration at times when such data collection is needed, but to transform into a less-obtrusive second configuration at times when such data collection is not needed.

In an example, a moveable sensor arm can be visually less-obtrusive in a second arm configuration because it is substantially hidden behind (from an external perspective) a face-spanning support member when the sensor arm is in the second arm configuration. In an example, a moveable sensor arm can be visually more-obtrusive in a first arm configuration because it moves out from behind (from an external perspective) a face-spanning support member when the sensor arm is in the first arm configuration.

In an example, a moveable sensor arm can be visually less-obtrusive in a second arm configuration because it is substantially aligned with the longitudinal axis of a face-spanning support member when the sensor arm is in the second arm configuration. In an example, a moveable sensor arm can be visually more-intrusive in a first arm configuration because it moves out of alignment with the longitudinal axis of a face-spanning support member when the sensor arm is in the first arm configuration.

In an example, a moveable sensor arm can be visually less-obtrusive in a second arm configuration because it fits within a recess, channel, and/or slot within a face-spanning support member when the sensor arm is in the second arm configuration. In an example, a moveable sensor arm can be visually more-intrusive in a first arm configuration because it moves out of the recess, channel, and/or slot.

In an example, a moveable sensor arm is changed from a first arm configuration to a second arm configuration, or vice versa, by being moved relative to a face-spanning support member. In an example, movement of a moveable sensor arm from a second arm configuration to a first arm configuration causes an electromagnetic energy sensor to be moved to a selected location from which the sensor collects data concerning electromagnetic brain activity. In an example, a moveable sensor arm is at least partially attached to a face-spanning support member in both a first arm configuration and a second arm configuration.

In an example, a moveable sensor arm can be connected to a face-spanning support member at a single point or by a single axle. In an example, a moveable sensor arm can be pivoted and/or rotated around this single point or single axle in order to transition from a first arm configuration to a second arm configuration, or vice versa. In an example, a single point or single axle connection between a moveable sensor arm and a face-spanning support member can be at an end-point of a moveable sensor arm. In an example, a moveable sensor arm can pivot around this single point or single axle. In an example, a single point or single axle connection between a moveable sensor arm and a face-spanning support member can be at a more-central location on a moveable sensor arm. In an example, a moveable sensor arm can rotate around this single point or single axle.

In an example, pivoting or rotating a moveable sensor arm can cause the arm to protrude out from a face-spanning support member in a manner which brings one or more electromagnetic energy sensors into contact with the surface of a person's head at a selected location. In an example, pivoting or rotating a moveable sensor arm can cause the arm to protrude out from a face-spanning support member in a manner which brings one or more electromagnetic energy sensors into electromagnetic communication with a person's head at a selected location. In an example, pivoting or rotating a moveable sensor arm can cause the arm to protrude upwards and bring an electromagnetic energy sensor into electromagnetic communication with a person's forehead and/or temples.

In an example, pivoting or rotating a moveable sensor arm can cause the arm to protrude out from a face-spanning support member in a manner which brings one or more electromagnetic energy sensors into contact with one or more locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a moveable sensor arm can be connected to a face-spanning support member at two or more locations and/or by two or more axles. In an example, a moveable sensor arm can be changed from a first arm configuration to a second arm configuration, or vice versa, by the relative movement of the two or more connection locations or axles. In an example, the distances between two or more connection locations or axles can be changed. In an example, changes in the distances between two or more connection locations or axles can change the shape of a moveable sensor arm. In an example, changes in the distances between two or more connection locations or axles can cause a moveable sensor arm to bend, flex, or fold. In an example, such bending, flexing, or folding can change a moveable sensor arm from a first arm configuration to a second arm configuration, or vice versa.

In an example, bending, flexing, or folding a moveable sensor arm can cause the arm to protrude out from a face-spanning support member in a manner which brings one or more electromagnetic energy sensors into contact with the surface of a person's head at a selected location. In an example, bending, flexing, or folding a moveable sensor arm can cause the arm to protrude out from a face-spanning support member in a manner which brings one or more electromagnetic energy sensors into electromagnetic communication with a person's head at a selected location. In an example, bending, flexing, or folding a moveable sensor arm can cause the arm to protrude upwards and bring an electromagnetic energy sensor into electromagnetic communication with a person's forehead and/or temples.

In an example, bending, flexing, or folding a moveable sensor arm can cause the arm to protrude out from a face-spanning support member in a manner which brings one or more electromagnetic energy sensors into contact with one or more locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a moveable sensor arm can be connected to a face-spanning support member such that the arm can slide along a track or channel. In an example, a moveable sensor arm can be changed from a first arm configuration to a second arm configuration, or vice versa, by sliding along this track or channel. In an example, sliding a moveable sensor arm can bring an electromagnetic energy sensor into electromagnetic communication with a person's forehead and/or temples. In an example, sliding a moveable sensor arm can bring one or more electromagnetic energy sensors into contact with one or more locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a moveable sensor arm can be connected to a face-spanning support member by a hinge mechanism. In an example, a moveable sensor arm can be changed from a first arm configuration to a second arm configuration, or vice versa, by movement of this hinge mechanism. In an example, movement of this hinge can bring an electromagnetic energy sensor into electromagnetic communication with a person's forehead and/or temples. In an example, movement of this hinge can bring one or more electromagnetic energy sensors into contact with one or more locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a moveable sensor arm can be moved from being substantially horizontal in a second arm configuration to being substantially vertical in a first arm configuration. In an example, a portion of a moveable sensor arm can be pivoted or rotated upwards from a face-spanning support in order to span a portion of a person's forehead and/or temples and bring an electromagnetic energy sensor into contact with the person's forehead and/or temples. In an example, a portion of a moveable sensor arm can be bent, flexed, or folded upwards from a face-spanning support in order to span a portion of a person's forehead and/or temples and bring an electromagnetic energy sensor into contact with the person's forehead and/or temples. In an example, a portion of a moveable sensor arm can be slid relative to a face-spanning support in order to span a portion of a person's forehead and/or temples and bring an electromagnetic energy sensor into contact with the person's forehead and/or temples.

In an example, a portion of a moveable sensor arm can be pivoted, rotated, bent, flexed, folded, or slid relative to a face-spanning support in order to span a portion of a person's head and bring an electromagnetic energy sensor into contact with one or more locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a moveable sensor arm can be substantially straight. In an example, a moveable sensor arm can be arcuate. In an example, a moveable sensor arm can have a shape which is a conic section. In an example, a moveable sensor arm can be an arc of a circle. In an example, a moveable sensor arm can be substantially perpendicular to the longitudinal axis of a face-spanning support member in a first arm configuration and can be substantially parallel to the longitudinal axis of a face-spanning support member in a second arm configuration. In an example, a moveable sensor arm can be an arcuate loop which loops over a person's forehead, over the top of the person's head, and/or around the back of the person's head.

In an example, a moveable sensor arm can change from a more arcuate shape in its first configuration to a less arcuate shape in its second configuration. In an example, a moveable sensor arm can change from a more convex shape in its first configuration to a less convex shape in its second configuration. In an example, moving the two ends of a moveable sensor arm closer together can cause the arm to protrude upwards onto a person's forehead and/or temple. In an example, a moveable sensor arm can be moved from a first arm configuration to a second arm configuration, or vice versa, along a path which is substantially parallel to the surface of a person's head. In an example, a moveable sensor arm can be moved from a first arm configuration to a second arm configuration, or vice versa, along a path which is a substantially constant distance from the arcuate surface of a person's head.

In an example, a moveable sensor arm can be configured to horizontally span a person's forehead and/or temple. In an example, a moveable sensor arm can be configured to loop over the top of a person's head. In an example, a moveable sensor arm can be configured to loop around the back of a person's head. In an example, this device can comprise two symmetric moveable sensor arms, one on the right side of a person's head and one on the left side of a person's head. In an example, this device can comprise a single moveable sensor arm on one side of a person's head.

In an example, a moveable sensor arm can be manually moved from a first arm configuration to a second arm configuration (and vice versa) by a person. In an example, a moveable sensor arm can be automatically moved from a first arm configuration to a second arm configuration (and vice versa) by an actuator which is manually activated by a person. In an example, a moveable sensor arm can be automatically moved from a first arm configuration to a second arm configuration (and vice versa) by an actuator which is automatically activated based on data from one or more wearable sensors. In an example, a moveable sensor arm can be automatically moved from a first arm configuration to a second arm configuration (and vice versa) by an actuator which is automatically activated by a data processing unit.

In an example, a moveable sensor arm can be rigid. In an example, a moveable sensor can be made from metal, a polymer, a textile, or a combination thereof. In an example, a moveable sensor arm can be sufficiently flexible to hold an electromagnetic energy sensor against the surface of a person's head. In an example, a moveable sensor arm can bend. In an example, a moveable sensor arm can be elastic, but sufficiently resistant to hold an electromagnetic energy sensor against the surface of a person's head. In an example, a moveable sensor arm can be kept in close contact with the surface of a person's head by means of a spring or an elastic member. In an example, a moveable sensor arm can keep an electromagnetic energy sensor in close contact with the surface of a person's head by means of a spring or an elastic member.

In an example, a moveable sensor arm can hold a single electromagnetic energy sensor in a selected location in order to collect data concerning electromagnetic brain activity. In an example, a moveable sensor arm can hold a plurality of electromagnetic energy sensors in selected locations in order to collect data concerning electromagnetic brain activity. In an example, the locations of one or more electromagnetic energy sensors relative to a person's head are changed by the movement of a moveable sensor arm relative to a face-spanning support member. In an example, the locations of one or more electromagnetic energy sensors relative to a face-spanning support member can be changed by the movement of a moveable sensor arm relative to a face-spanning support member. In an example, the locations of one or more electromagnetic energy sensors can be changed when a moveable sensor arm is moved from a first arm configuration to a second arm configuration.

In an example, a device can further comprise one or more components selected from the group consisting of: data processor, power source, data communication component, human-to-computer user interface, computer-to-human interface, digital memory, one or more additional wearable sensors, and an external electromagnetic energy emitter. In an example, one or more of the components selected from this group can be connected to, attached to, and/or integrated into the face-spanning support member.

In an example, a data processor can perform one or more functions selected from the group consisting of: convert analog sensor signals to digital signals, filter sensor signals, amplify sensor signals, analyze sensor data, run software programs, and store data in memory. In an example, a data processor can analyze data using one or more statistical methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; AI (e.g. machine learning); power spectral density; power spectrum analysis; probit model; and time-series analysis.

In an example, a power source can be a battery. In an example, a power source can harvest, transduce, or generate electrical energy from kinetic energy, thermal energy, biochemical energy, ambient light energy, and/or ambient electromagnetic energy. In an example, a power source can comprise: power from a source that is internal to the device during regular operation (such as an internal battery, capacitor, energy-storing microchip, wound coil or spring); power that is obtained, harvested, or transduced from a source other than a person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from a person's body (such as kinetic or mechanical energy from body motion, electromagnetic energy from a person's body, or thermal energy from a person's body).

In an example, a data communication component can perform one or more functions selected from the group consisting of: transmit and receive data via Bluetooth, WiFi, Zigbee, or other wireless communication modality; transmit and receive data to and from a home appliance and/or home control system; transmit and receive data to and from a mobile electronic device such as a cellular phone, mobile phone, smart phone, electronic tablet; transmit and receive data to and from a separate wearable device such as a smart watch or smart clothing; transmit and receive data to and from the internet; send and receive phone calls and electronic messages; and transmit and receive data to and from an implantable medical device.

In an example, a data communication component can be in wireless communication with a separate mobile device selected from the group consisting of: smart phone, mobile phone, holophone, or cellular phone; PDA; electronic tablet; electronic pad; and other electronically-functional handheld device. In an example, a data communication component can be in wireless communication with a relatively fixed-location device selected from the group consisting of: laptop computer, desktop computer, internet terminal, smart appliance, home control system, and other fixed-location electronic communication device. In an example, a data communication component can communicate with one or more other devices selected from the group consisting of: a communication tower or satellite; a CPAP device and/or respiratory mask; an appliance, home environment control system, and/or home security system; a laptop or desktop computer; a smart phone or other mobile communication device; a wearable cardiac monitor; a wearable electromagnetic brain activity monitor; a wearable pulmonary activity monitor; an implantable medical device; an internet server; and another type of wearable device or an array of wearable sensors.

In an example, a human-to-computer interface can further comprise one or more members selected from the group consisting of: buttons, knobs, dials, or keys; display screen; gesture-recognition interface; microphone; physical keypad or keyboard; virtual keypad or keyboard; speech or voice recognition interface; touch screen; EMG-recognition interface; and EEG-recognition interface. In an example, a device can comprise a device and method that enables a person to control a computer or other electronic device by modifying their electromagnetic brain activity. In an example, a device can comprise a device and method that enables a health care provider to better diagnose a health condition related to electromagnetic brain activity.

In an example, a computer-to-human interface can further comprise one or more members selected from the group consisting of: a display screen; a speaker or other sound-emitting member; a myostimulating member; a neurostimulating member; a speech or voice recognition interface; a synthesized voice; a vibrating or other tactile sensation creating member; MEMS actuator; an electromagnetic energy emitter; an infrared light projector; an LED or LED array; and an image projector.

In an example, a device can comprise a device and method for neurofeedback. In an example, the person for whom data concerning their electromagnetic brain activity is collected by one or more electromagnetic energy sensors can receive feedback based on analysis of that data. In an example, this feedback can be in real time. In an example, this device can help a person to self-modify their electromagnetic brain activity in order to improve their mental functioning and/or health status.

In an example, a device can comprise eyewear that transmits and/or displays a combination of environmental objects and virtual objects. In an example, a person can modify and control the combination of environmental and virtual objects which they see by changing their brainwave patterns. In an example, a person can alter the relative proportion of environmental content vs. virtual content in an augmented reality system based on changes in their electromagnetic brain activity. In an example, a person can alter and control the type of virtual content which is combined with environmental content by changing their brain activity. In various examples, a device can comprise eyewear selected from the group consisting of: non-prescription eyeglasses, prescription eyeglasses, sunglasses, goggles, contact lenses, visor, monocle, eyewear-based human-to-computer interface, eyeglasses with integrated camera, augmented reality (AR) glasses, and virtual reality (VR) glasses.

In an example, one or more additional wearable sensors can be selected from the group consisting of: motion sensor, inertial sensor, single axis, biaxial, or multi-axial accelerometer, kinematic sensor, gyroscope, tilt sensor, inclinometer, vibration sensor, bend sensor, goniometer, strain gauge, stretch sensor, pressure sensor, force sensor, flow sensor, air pressure sensor, altimeter, barometer, blood flow monitor, blood pressure monitor, microcantilever sensor, microfluidic sensor, peak flow meter, nanotube sensor, gesture recognition sensor, global positioning system (GPS) module, and compass.

In an example, one or more additional sensors can be selected from the group consisting of: light energy sensor, ambient light sensor, electro-optical sensor, infrared sensor, laser sensor, light intensity sensor, optical sensor, optoelectronic sensor, photochemical sensor, photoelectric sensor, photometer, ultraviolet light sensor, chemiluminescence sensor, image recorder, camera, video recorder, spectroscopic sensor, light-spectrum-analyzing sensor, color sensor, spectral analysis sensor, spectrometry sensor, spectrophotometric sensor, spectroscopy sensor, near-infrared, infrared, ultraviolet, or white light spectroscopy sensor, mass spectrometry sensor, Raman spectroscopy sensor, ion mobility spectroscopic sensor, chromatography sensor, optical glucose sensor, gas chromatography sensor, and analytical chromatography sensor. In an example, one or more additional sensors can be selected from the group consisting of: sound sensor, sonic energy sensor, microphone, speech and/or voice recognition interface, breathing sound monitor, chewing and/or swallowing monitor, ambient sound sensor or monitor, and ultrasound sensor.

In an example, one or more additional sensors can be selected from the group consisting of: temperature and/or thermal energy sensor, thermistor, thermometer, thermopile, body temperature sensor, skin temperature sensor, ambient temperature sensor, biochemical sensor, ambient air monitor, amino acid sensor, artificial olfactory sensor, blood glucose monitor, blood oximeter, body fat sensor, capnography sensor, carbon dioxide sensor, carbon monoxide sensor, cerebral oximetry monitor, chemical sensor, chemiresistor sensor, chemoreceptor sensor, cholesterol sensor, cutaneous oxygen monitor, ear oximeter, food identification sensor, food consumption monitor, caloric intake monitor, gas composition sensor, glucometer, glucose monitor, humidity sensor, hydration sensor, microbial sensor, moisture sensor, osmolality sensor, oximeter, oximetry sensor, oxygen consumption monitor, oxygen level monitor or sensor, oxygen saturation monitor, pH level sensor, porosity sensor, pulse oximeter, skin moisture sensor, sodium sensor, tissue oximetry sensor, and tissue saturation oximeter.

In an example, an external electromagnetic energy emitter can transmit electromagnetic energy into the surface of a person's body. In an example, an external electromagnetic energy emitter can be used in combination with an electromagnetic energy sensor in order to measure the electromagnetic conductivity, resistance, and/or impedance of body tissue. In an example, an external electromagnetic energy emitter can transmit electromagnetic energy into body tissue in order to modify, adjust, stimulate, and/or block electromagnetic brain activity. In an example, an external electromagnetic energy emitter can transmit electromagnetic energy into body tissue in order to modify, adjust, stimulate, and/or block peripheral nervous system activity. In an example, an external electromagnetic energy emitter can transmit electromagnetic energy into body tissue in order to modify, adjust, stimulate, and/or block muscular activity. In an example, an external electromagnetic energy emitter can be a neurostimulator or myostimulator.

In an example, electromagnetic energy which is emitted from an external electromagnetic energy emitter can be selectively adjusted. In various examples, adjustable parameters of transmitted electromagnetic energy can be selected from the group consisting of: the particular wave form or wave morphology (e.g. sinusoidal wave, saw tooth wave, square wave, triangle wave, biphasic pattern, tri-phasic pattern, signal spikes, pattern randomization, pattern repetition, Fourier transformation parameter, pattern mimicking a natural neural transmission signal, and pattern inverting a natural neural transmission signal), wave or pulse frequency (e.g. in the range of 0.1 Hz to 2,500 Hz), wave or pulse amplitude (e.g. in the range from 1 ?A to 1000 mA), wave or pulse width (e.g. in the range of 5 ?Sec to 500 mSec), electrical current level (e.g. in the range from 0.01 mA to 1000 mA), electromagnetic field (e.g. in the range of 5 V/m to 500 V/m), electromagnetic field gradient (e.g. over 1 V/m/mm), signal continuity and duty cycle, signal cycling times, signal ramping, and signal dampening.

In an example, a device can be embodied in a shape-transforming eyewear device for collecting data concerning electromagnetic brain activity comprising: a face-spanning support member which is configured to span an upper portion of a person's face; at least one optical member which transmits light from the person's environment and/or light from a virtual image display to at least one of the person's eyes; an electromagnetic energy sensor, wherein this electromagnetic energy sensor has a first sensor configuration wherein it is configured to be at a selected location relative to the surface of the person's head in order to collect data concerning electromagnetic brain activity and wherein this electromagnetic energy sensor has a second sensor configuration wherein it is not at this selected location; and a movable sensor arm which holds the electromagnetic energy sensor and is at least partially connected to the face-spanning support member, wherein this moveable sensor arm has a first arm configuration which holds the electromagnetic energy sensor in the first sensor configuration, wherein this moveable sensor arm has a second arm configuration which holds the electromagnetic energy sensor in the second sensor configuration, and wherein this moveable sensor arm is moved relative to the face-spanning support member in order to change from the first arm configuration to the second arm configuration, or vice versa.

In an example, a device can have a moveable sensor arm. In a first configuration, an electromagnetic energy sensor is at a first location from which it collects data concerning electromagnetic brain activity. In a second configuration, the electromagnetic energy sensor is at a second location. In an example, a location from which an electromagnetic energy sensor collects data concerning electromagnetic brain activity can be selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a face-spanning support member can be a single-piece member which spans from one ear to the other. In an example, a face-spanning support member can have two side portions (which each span from an ear to the front of the face) and a front portion (which spans the face from one side to the other). In an example, the side portions can be horizontally aligned with a person's eyes and the front portion can be horizontally aligned with the person's eyebrows. In an example, optical members can be lenses. In an example, a face-spanning support member and optical members can together comprise a pair of electronically-functional eyeglasses. In an example, the selected location from which an electromagnetic energy sensor collects data concerning electromagnetic brain activity can be on a person's forehead and/or temple.

In an example, a moveable sensor arm can be visually less-obtrusive in a second arm configuration than in a first arm configuration. Thus, this eyewear can collect data concerning electromagnetic brain activity using a more-obtrusive configuration at times when such data collection is needed, but can transform into a less-obtrusive configuration at times when such data collection is not needed. This enables a person to wear a mobile EEG monitor without looking too dorky—or at least without looking dorky all the time. The moveable sensor arm can be visually less-obtrusive in the second arm configuration because it is substantially hidden behind (interior to) the side portion of a face-spanning support member.

In an example, a moveable sensor arm can pivot and/or rotate around an axle in order to change from a first arm configuration to a second arm configuration, or vice versa. In an example, an axle can be located near an ear. In an example, in the second arm configuration, a moveable sensor arm can span forward from an axle toward the front of a person's face. In an example, a moveable sensor arm can be slightly arcuate. In another example, a moveable sensor arm can be straight. In an example, a moveable sensor arm can be moved manually. In another example, a moveable sensor arm can be moved automatically by an actuator.

In an example, a moveable sensor arm can pivot and/or rotate around an axle in order to change from a first arm configuration to a second arm configuration, or vice versa. In an example, an axle can be located near the middle of the front portion of a face-spanning support member. In an example, in the second arm configuration, the moveable sensor arm can span sideways from the axle. In an example, a moveable sensor arm can be straight. In another example, a moveable sensor arm can be arcuate. In an example, a moveable sensor arm can be moved manually. In another example, a moveable sensor arm can be moved automatically by an actuator. In an example, there is one central moveable sensor arm and one central electromagnetic energy sensor. In another example, there can be two moveable sensor arms and two electromagnetic energy sensors, one on each side of the forehead and/or one above each of the two optical members.

In an example, a device can be embodied in a shape-transforming eyewear device for collecting data concerning electromagnetic brain activity comprising: a face-spanning support member which is configured to span an upper portion of a person's face; at least one optical member which transmits light from the person's environment and/or light from a virtual image display to at least one of the person's eyes; one or more electromagnetic energy sensors, wherein these electromagnetic energy sensors have a first sensor configuration wherein they are configured to be at a selected locations relative to the surface of the person's head in order to collect data concerning electromagnetic brain activity and wherein these electromagnetic energy sensors have a second sensor configuration wherein they are not at these selected locations; and a movable sensor arm which holds the electromagnetic energy sensors and is at least partially connected to the face-spanning support member, wherein this moveable sensor arm has a first arm configuration which holds the electromagnetic energy sensors in the first sensor configuration, wherein this moveable sensor arm has a second arm configuration which holds the electromagnetic energy sensors in the second sensor configuration, and wherein this moveable sensor arm is moved relative to the face-spanning support member in order to change from the first arm configuration to the second arm configuration, or vice versa.

In an example, in a second arm configuration, a moveable sensor arm can be substantially aligned with, hidden behind, and interior to face-spanning support member. In an example, moveable sensor arm can be bent, flexed, and/or folded upwards from a face-spanning support member when the distance between connectors is decreased. In an example, the distance between connectors is decreased when a first connector is moved toward a second connector. In an example, this movement can be along a track and/or channel in a face-spanning support member. In another example, both connectors can be moved toward each other. In an example, when a moveable sensor arm is bent, flexed, and/or folded upwards into a first arm configuration, it can move an electromagnetic energy sensor to a selected location on a person's temple and/or forehead from which this sensor collects data concerning electromagnetic brain activity.

In an example, a connector can be located near the front end of a side portion of face-spanning support member. In an example, a connector can be located near the rear end of a side portion of a face-spanning support member in a second arm configuration and near the middle of the side portion of face-spanning support member in a first arm configuration. In an example, the device can be symmetric. In an example, a moveable sensor arm can be moved manually. In another example, a moveable sensor arm can be moved automatically by an actuator.

In another example, a face-spanning support member can be a single-piece member which spans a portion of the circumference of a person's head. In an example, a face-spanning support member can span from one ear to the other ear in a substantially-horizontal manner. In an example, face-spanning support member can have a longitudinal axis which spans a portion of a person's head in a substantially-horizontal manner. In an example, when a person is standing up, a plane formed by this longitudinal axis can intersect a horizontal plane at an angle which is less than 45 degrees. In an example, face-spanning support member can span a person's face at a horizontal level which is substantially aligned with the person's eyebrows. In another example, a face-spanning support member can span a person's face across the person's forehead. In an example, a face-spanning support member can span the sides of a person's head at levels which are proximal to the upper portions of the person's ears.

In an example, a moveable sensor arm can be moved along the longitudinal axis of a face-spanning support member from a first arm configuration to a second arm configuration, and vice versa. In an example, a moveable sensor arm can slide along a track and/or channel on face-spanning support member. In an example, a moveable sensor arm can be located on a person's forehead in the first arm configuration and be located on the side or back of the person's head in a second arm configuration. In an example, there can be one moveable sensor arm and one central electromagnetic energy sensor. In another example, there can be a plurality of moveable sensor arms and electromagnetic energy sensors. In an example, a moveable sensor arm can be moved manually. In another example, a moveable sensor arm can be moved automatically by an actuator.

In an example, a face-spanning support member can be a single-piece member which spans the circumference of a person's head in a substantially-horizontal manner. In an example, a face-spanning support member can have a longitudinal axis which encircles a person's head in a substantially-horizontal manner. In an example, when a person is standing up, a plane formed by this longitudinal axis can intersect a horizontal plane at an angle which is less than 45 degrees. In an example, a face-spanning support member can span a person's face at a horizontal level which is substantially aligned with the person's eyebrows. In another example, a face-spanning support member can span a person's face across the person's forehead. In an example, a face-spanning support member can span the sides of the person's head at levels which are proximal to the upper portions of the person's ears.

In an example, a face-spanning support member can be a single-piece member which spans a portion of the circumference of a person's head. In an example, a face-spanning support member can span from one ear to the other ear in a substantially-horizontal manner. In an example, face-spanning support member can have a longitudinal axis which spans a portion of a person's head in a substantially-horizontal manner. In an example, when a person is standing up, a plane formed by this longitudinal axis can intersect a horizontal plane at an angle which is less than 45 degrees. In an example, face-spanning support member can span a person's face at a horizontal level which is substantially aligned with the person's eyebrows. In another example, a face-spanning support member can span a person's face across the person's forehead. In an example, a face-spanning support member can span the sides of a person's head at levels which are proximal to the upper portions of the person's ears.

In an example, a face-spanning support member can be a single-piece member which spans a portion of the circumference of a person's head. In an example, a face-spanning support member can span from one ear to the other ear in a substantially-horizontal manner. In an example, face-spanning support member can have a longitudinal axis which spans a portion of a person's head in a substantially-horizontal manner. In an example, when a person is standing up, a plane formed by this longitudinal axis can intersect a horizontal plane at an angle which is less than 45 degrees. In an example, a face-spanning support member can span a person's face at a horizontal level which is substantially aligned with the person's eyebrows. In another example, a face-spanning support member can span a person's face across the person's forehead. In an example, a face-spanning support member can span the sides of a person's head at levels which are proximal to the upper portions of the person's ears.

In an example, a moveable sensor arm can pivot and/or rotate around an axle in order to change from a second arm configuration to a first arm configuration, or vice versa. In an example, a moveable sensor arm can be moved manually. In another example, moveable sensor arm can be moved automatically by an actuator.

In example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears; at least one forehead ascending member which is attached to or an integrated part of the lateral ear support member which is configured to span at least a portion of the person's temple and/or forehead above the lateral ear support member; at least one electromagnetic energy sensor which is attached to or an integrated part of the forehead ascending member and which is configured to receive electromagnetic energy from the person's brain; and a control unit with which the at least one electromagnetic energy sensor is in electromagnetic communication.

In an example, a device can further comprise a reference electrode which is in electromagnetic communication with a person's head in proximity to the person's ear. In an example, a device can be symmetric with respect to the sides of the person's head, with similar, symmetric components assumed on both the right and left sides of the person's head. In an example, some of the components may not be symmetric with respect to the right and left sides of the person's head. In an example, there can be a forehead ascending member (and associated electromagnetic sensors) on both sides of the person's head. In an example, there can be a forehead ascending member (and associated electromagnetic sensors) only on one side of the person's head.

In an example, a frontal support member can longitudinally and horizontally span a portion of the person's face within 1 of the person's eyebrows. In an example, a frontal support member can be straight. In an example, a frontal support member can be arcuate. In an example, a frontal support member (or projections from it) can rest on the bridge of the person's nose. In an example, a frontal support member can be the front portion of the frame of a pair of eyeglasses.

In an example, an optical member can be an optical lens which modifies light rays from an object in the environment before these light rays are received by an eye. In an example, an optical member can be a convex or concave lens. In an example, an optical member can be an image-displaying member which creates a virtual image which is seen by an eye. In an example, an optical member can be a display screen. In an example, an optical member can be a composite optical member which allows light rays from an object in the environment to reach an eye and also creates a virtual image which is seen by the eye. In an example, an optical member can be part of a virtual reality or augmented reality system.

In an example, at least one lateral ear support member can be connected to a frontal support member by a hinge and/or spring mechanism. In an example, at least one lateral ear support member can be an integrated part (one continuous piece) of the frontal support member. In an example, a lateral ear support member can be an ear piece or side frame of a pair of eyeglasses. In an example, a lateral ear support member can be straight. In an example, a lateral ear support member can be arcuate. In an example, a distal end of a lateral ear support member can rest on top of a person's ear. In an example, a distal end of lateral ear support member can curve around a person's ear.

In an example, a forehead ascending member can be attached to (and/or branch off from) a lateral ear support member at a location in a middle section of the lateral ear support member. In an example, a forehead ascending member can be attached to (and/or branch off from) a lateral ear support member at a location in the middle section of the lateral ear support member which at least ½ inch from the frontal support member. In an example, a forehead ascending member can be attached to (and/or branch off from) a lateral ear support member at a location in a middle section of the lateral ear support member which at least ½ inch from the distal end of the lateral ear support member. In an example, a forehead ascending member can be attached to (and/or branch off from) a lateral ear support member at a location in a middle section of the lateral ear support member which at least ½ inch from the top of the person's ear.

In an example, a forehead ascending member can span a distance between ¼ inch and 6 inches. In an example, a forehead ascending member can be arcuate. In an example, a forehead ascending member can have an arcuate shape which branches off from the lateral ear support member at an acute angle (X), diverges from the lateral ear support member at a growing angle (Y, which is greater than X), and then further diverges at a shrinking angle (Z, which is less than X). In an example, a forehead ascending member can have an arcuate shape which comprises a portion of a sine curve. In an example, a forehead ascending member can have an arcuate shape which is a conic section. In an example, a forehead ascending member can branch off from a lateral ear support member and ascend to a person's temple. In an example, a forehead ascending member can branch off from a lateral ear support member and ascend to a location on a person's forehead which is directly above the person's eye. In an example, a forehead ascending member can branch off from a lateral ear support member and ascend to the center of the person's forehead. In an example a forehead ascending member can ascend to a location on a person's forehead which is between ¼ and 4 above the person's eyebrows.

In an example, a forehead ascending member can have a lower end which branches off from the lateral ear support member and an upper end which is located over the person's temple or forehead, wherein projecting and plotting the locations of the lower and upper ends onto a vertical plane yields a linear approximation of the slope of the forehead ascending member in the range of 0.5 to 2. In an example, a forehead ascending member can have a lower end which branches off from the lateral ear support member and an upper end which is located over the person's temple or forehead, wherein projecting and plotting the locations of the lower and upper ends onto a vertical plane yields a linear approximation of the upward angle of the forehead ascending member in the range of 30 degrees to 150 degrees.

In an example, a forehead ascending member can travel along an inward (closer to the face) as well as an upward (closer to the top of the head) path after branching off from a lateral ear support member. In an example, a forehead ascending member can have a spring mechanism which keeps it close to (and/or exerts pressure on) the surface of the person's head. In an example, a forehead ascending member can further comprise an inflatable member which keeps it close to (and/or exerts pressure on) the surface of the person's head. In an example, a forehead ascending member can have a motion mechanism which helps to keep it close to (and/or exerts pressure on) the surface of the person's head. In an example this motion mechanism can be selected from the group consisting of: electric motor or actuator; MEMS; pneumatic member; hydraulic member; spring; and other tensile member.

In an example, an electromagnetic energy sensor can be an electroencephalographic (EEG) sensor. In an example, data collected by an electromagnetic energy sensor can be used to measure electromagnetic energy emitted by a person's brain. In an example, data collected by electromagnetic energy sensors can be used to measure a person's brain waves. In an example, data from the sensors can be analyzed using Fourier Transformation to identify wave patterns at different frequencies or within selected frequency ranges. In an example, the relative power of brain waves in different frequency ranges can be tracked and analyzed.

In an example, an electromagnetic energy sensor can be a dry EEG sensor which does not require gel or liquid to make electromagnetic contact with a person's brain. In an example, an electromagnetic energy sensor can be in direct physical contact with the person's skin. In an example, an electromagnetic energy sensor can be in electromagnetic communication with the person's brain without being in direct physical contact with the person's skin. In an example, there can be two electromagnetic energy sensors on one forehead ascending member, one closer to the person's ear and one further up toward the center of the person's forehead. In an example, there can be only one electromagnetic energy sensor on a forehead ascending member. In an example, there can be three or more electromagnetic energy sensors on a forehead ascending member.

In an example, a control unit can further comprise a power source, a data processing unit, a human-to-computer control interface, a computer-to-human interface, and a wireless data transmitter. In an example, one or more electromagnetic energy sensors can be in electromagnetic communication with the control unit. In an example, data from one or more electromagnetic energy sensors can be analyzed within a data processing unit within the control unit. In an example, data from one or more electromagnetic energy sensors can be wirelessly transmitted to a remote data processing unit and analyzed within that remote data processing unit. In an example, a power source can be a battery. In an example, a power source can further comprise an energy harvesting member which transduces kinetic, thermal, and/or ambient electromagnetic energy into power for the device. In an example, a human-to-computer control interface can be selected from the group consisting of: voice or speech recognition; a button or other touch-based control; and gesture recognition. In an example, data from the electromagnetic energy sensors themselves can be analyzed and used as part of the human-to-computer control interface for the device.

In an example, a forehead ascending member can stop near a person's temple and only hold one electromagnetic energy sensor. In an example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears; at least one forehead ascending member which is attached to or an integrated part of the lateral ear support member which is configured to span at least a portion of the person's temple and/or forehead above the lateral ear support member; an electromagnetic energy sensor which is attached to or an integrated part of the forehead ascending member and which is configured to receive electromagnetic energy from the person's brain; and a control unit with which electromagnetic energy sensor is in electromagnetic communication. In an example, a device can further comprise a reference electrode which is in electromagnetic communication with a person's head in proximity to the person's ear.

In an example, a forehead ascending member can stop near a person's temple and form a curve which is connected to a lateral ear support member at two locations. In example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears; at least one arcuate forehead ascending member which is attached to the lateral ear support member at two locations and which is configured to span at least a portion of the person's temple and/or forehead above the lateral ear support member; an electromagnetic energy sensor which is attached to or an integrated part of the forehead ascending member and which is configured to receive electromagnetic energy from the person's brain; and a control unit with which the at least one electromagnetic energy sensor is in electromagnetic communication. In an example, a device can further comprise a reference electrode which is in electromagnetic communication with a person's head in proximity to the person's ear.

In an example, a device may not have a separate forehead ascending member. Instead, a middle section of a lateral ear support member can curve upwards near a person's temple and/or forehead to hold an electromagnetic energy sensor there. In an example, an eyewear device which collects data concerning electromagnetic energy from a person's brain comprising: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears, wherein a middle section of this lateral ear support member extends upward to span at least a portion of the person's temple and/or forehead; an electromagnetic energy sensor which is attached to or an integrated part of the middle section of lateral ear support member and which is configured to receive electromagnetic energy from the person's brain; and a control unit with which the at least one electromagnetic energy sensor is in electromagnetic communication. In an example, a device can further comprise a reference electrode which is in electromagnetic communication with a person's head in proximity to the person's ear.

In an example, a middle section of a lateral ear support can curve upwards in a sinusoidal manner. In an example, a middle section of a lateral ear support which is more than ½ inch from a frontal support member and more than ½ inch from the top of the person's ear can curve upwards in a sinusoidal manner to span a portion of a person's temple and/or forehead. In an example, this middle section can be shaped like a conic section. In an example, a lateral ear support can be generally straight except for a central portion which curves and/or loops upward to hold an electromagnetic energy sensor near a person's temple. In an example, a lateral ear support can be generally straight except for a central portion which curves and/or loops upward to hold one or more electromagnetic energy sensors over one or more locations on the side of a person's forehead. In an example, a lateral ear support can be a longitudinally undulating member with a substantially sinusoidal shape spanning between a person's ear and a frontal support member, wherein one or more electromagnetic energy sensors are located along the upper portions of this sinusoidal shape.

In an example, a middle section of a lateral ear support can hold one or more electromagnetic energy sensors in locations which are at least ¼ higher than a frontal support member. In an example, a middle section of a lateral ear support can hold one or more electromagnetic energy sensors in locations which are at least ½ higher than a frontal support member. In an example, a middle section of a lateral ear support can hold one or more electromagnetic energy sensors in locations which are at least ¼ higher than the top of a person's ear. In an example, a middle section of a lateral ear support can hold one or more electromagnetic energy sensors in locations which are at least ½ higher than the top of a person's ear.

In an example, a control unit can be located behind a person's ear. In an example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears, wherein a middle section of this lateral ear support member extends upward to span at least a portion of the person's temple and/or forehead; an electromagnetic energy sensor which is attached to or an integrated part of the middle section of lateral ear support member and which is configured to receive electromagnetic energy from the person's brain; and a control unit with which the at least one electromagnetic energy sensor is in electromagnetic communication. In an example, a device can further comprise a reference electrode which is in electromagnetic communication with the person's head in proximity to the person's ear.

In an example, a lateral ear support member can be attached to (or a single piece with) an encircling support member which goes around the back of the person's head (to a lateral ear support on the other side of the person's head). In an example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears, wherein a middle section of this lateral ear support member extends upward to span at least a portion of the person's temple and/or forehead; an electromagnetic energy sensor which is attached to or an integrated part of the middle section of lateral ear support member and which is configured to receive electromagnetic energy from the person's brain; an encircling support member which is attached to or an integrated part of the lateral ear support member and is configured to partially encircle the back of the person's head; an electromagnetic energy sensor which is attached to or an integrated part of the encircling support member and which is configured to receive electromagnetic energy from the person's brain; and a control unit with which the at least one electromagnetic energy sensor is in electromagnetic communication.

In an example, a device can comprise a member which branches off from a lateral ear support member in a descending manner (to hold an electromagnetic energy sensor on or in the ear), rather than an ascending manner. In an example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears; at least one descending member which is attached to or an integrated part of the lateral ear support member which is configured to span at least a portion of the person's ear; an electromagnetic energy sensor which is attached to or an integrated part of the descending member and which is configured to receive electromagnetic energy from the person's brain from a location on the person's ear, within the person's ear, and/or within 1 of the person's ear; and a control unit with which electromagnetic energy sensor is in electromagnetic communication.

In an example, a forehead ascending member can span the entire width of the person's forehead and connect to a symmetric forehead ascending member on the other side of the person's head. In an example, an eyewear device which collects data concerning electromagnetic energy from a person's brain can comprise: a frontal support member which is configured to be worn on a person's head and to span from one eye to the other eye across a portion of the front of the person's face; at least one optical member which is configured to be worn within 6 of one of the person's eyes and which is attached to or an integrated part of the frontal support member; at least one lateral ear support member which is attached to or an integrated part of the frontal support member and which is configured to be worn on or around one of the person's ears; at least one forehead ascending and spanning member which is attached to or an integrated part of the lateral ear support member which is configured to span the entire width of the person's forehead; electromagnetic energy sensors which are attached to or an integrated part of the forehead ascending and spanning member and which are configured to receive electromagnetic energy from the person's brain; and a control unit with which electromagnetic energy sensors are in electromagnetic communication. In an example, a device can further comprise a reference electrode which is in electromagnetic communication with a person's head in proximity to the person's ear.

In an example, a support member that spans the entire width of a person's forehead can enable a greater range of options for placement of electromagnetic energy sensors to more fully measure electromagnetic energy from a person's brain. In an example, a support member than spans the entire width of a person's forehead can hold four or more electromagnetic energy sensors. In an example, a support member which spans the entire width of a person's forehead can be a different (less obvious) color than a frontal support member. In an example, a support member that spans the entire width of a person's forehead can be transparent or translucent.

In an example, a device can be embodied in a mobile wearable electromagnetic brain activity monitor comprising: a wearable frame which is configured to be worn on a person's head; a plurality of electromagnetic energy sensors which are configured to be held in electromagnetic communication with the person's brain by the wearable frame, wherein these sensors collect data concerning the person's electromagnetic brain activity; and a control unit with one or more components selected from the group consisting of power source and/or power-transducing component, data transmission and data reception component, data memory component, and data processor.

In an example, the wearable frame can be a ring which is configured to encircle the person's head. In an example, the wearable frame can be substantially circular or elliptical and wherein this wearable frame can be configured to encircle the person's head at an anterior acute angle in the range of 0 to 45 degrees with respect to a horizontal plane when the person's head is upright. In an example, the wearable frame can be substantially circular or elliptical and wherein this wearable frame can be configured to span both the person's forehead and the rear of the person's head. In an example, the wearable frame can be a headband. In an example, the wearable frame can be an arcuate element which is configured to loop around the person's head from one ear to the other ear. In an example, the wearable frame can be a set of headphones.

In an example, the wearable frame can be configured to span from the person's left ear to the person's face, then span across the front of the person's face including a portion of the person's forehead, and then span from the person's face to the person's right ear. In an example, the wearable frame can be configured to rest on the bridge of the person's nose and on the person's ears. In an example, the wearable frame can be shaped like an eyeglasses and/or eyewear frame with the addition of extended elements around the ears. In an example, the wearable frame can be an eyeglasses and/or eyewear frame. In an example, the wearable frame can hold one or more light-transmitting optical members. In an example, the wearable frame can hold one or more optical lenses.

In an example, the plurality of electromagnetic energy sensors can be electroencephalogram (EEG) electrodes. In an example, the plurality of electromagnetic energy sensors can be configured to be worn less than one inch from the surface of the person's head. In an example, a power source and/or power-transducing component can be selected from the group consisting of: a power source that is internal to the device during regular operation, an internal battery, capacitor, energy-storing microchip, or wound coil or spring; a component for obtaining, harvesting, or transducing power from a source other than the person's body that is external to the device, a rechargeable battery, electromagnetic inductance from external source, solar energy, indoor lighting energy, wired connection to an external power source, ambient or localized radiofrequency energy, or ambient thermal energy; and a component for obtaining, harvesting, or transducing power from the person's body, as kinetic or mechanical energy from body motion, electromagnetic energy from the person's body, or thermal energy from the person's body.

In an example, a device can further comprise one or more interface components selected from the group consisting of: a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; one or more accelerometers; one or more cameras; and a GPS component.

In an example, a device can be embodied in a mobile wearable electromagnetic brain activity monitor comprising: an eyeglasses and/or eyewear frame which is configured to be worn on a person's head; a plurality of electromagnetic energy sensors which are configured to be held in electromagnetic communication with the person's brain by the eyeglasses and/or eyewear frame, wherein these sensors collect data concerning the person's electromagnetic brain activity; and a control unit with one or more components selected from the group consisting of power source and/or power-transducing component, data transmission and data reception component, data memory component, and data processor.

In an example, a device can be embodied in a mobile wearable electromagnetic brain activity monitor comprising: a headband or set of headphones which is configured to be worn on a person's head; a plurality of electromagnetic energy sensors which are configured to be held in electromagnetic communication with the person's brain by the headband or set of headphones, wherein these sensors collect data concerning the person's electromagnetic brain activity; and a control unit with one or more components selected from the group consisting of power source and/or power-transducing component, data transmission and data reception component, data memory component, and data processor.

In an example, a device can be embodied in a mobile wearable electromagnetic brain activity monitor comprising: a wearable frame which is configured to be worn on a person's head, wherein this wearable frame is configured to completely encircle a portion of the person's head, wherein this wearable frame has a central circular axis which is configured at an anterior acute angle in the range of 0 to 45 degrees with respect to a horizontal plane when the person's head is upright; a plurality of electromagnetic energy sensors which are configured to be held in electromagnetic communication with the person's brain by the wearable frame, wherein these sensors collect data concerning the person's electromagnetic brain activity; and a control unit with one or more components selected from the group consisting of power source and/or power-transducing component, data transmission and data reception component, data memory component, and data processor.

In an example, a device can be embodied in a mobile wearable electromagnetic brain activity monitor comprising: a wearable frame which is configured to be worn on a person's head, wherein this wearable frame is configured to span from one ear to the other ear across the front of the person's head; a plurality of electromagnetic energy sensors which are configured to be held in electromagnetic communication with the person's brain by the wearable frame, wherein these sensors collect data concerning the person's electromagnetic brain activity; and a control unit with one or more components selected from the group consisting of power source and/or power-transducing component, data transmission and data reception component, data memory component, and data processor.

In an example, the shape of the wearable frame can have sinusoidal variation around its central circular axis. In an example, the shape of the wearable frame can be configured to have an arcuate wave above an ear. In an example, the wearable frame can be configured to be supported by the person's ears. In an example, the monitor can further comprise at least one optical lens or other light-transmitting member. In an example, data from the electromagnetic energy sensors can be used to adjust and/or control the light absorption, light reflection, light refraction, light spectrum transformation, focal direction, focal distance, light polarization, and/or parallax view of the at least one optical lens or other light-transmitting member.

In an example, ae wearable frame can have a sensor-holding upward arcuate member which is configured to span a portion of the person's temple. In an example, the wearable frame can have a sensor-holding member which can be moved relative to the rest of the frame, wherein this sensor-holding member can be moved from a first configuration in which it does not span a portion of the person's temple to a second configuration in which it does span a portion of the person's temple.

In an example, a wearable frame can have a sensor-holding upward arcuate member which is configured to span a portion of the person's forehead. In an example, the wearable frame can have a sensor-holding member which can be moved relative to the rest of the frame, wherein this sensor-holding member can be moved from a first configuration in which it does not span a portion of the person's forehead to a second configuration in which it does span a portion of the person's forehead.

In an example, a monitor can further comprise one or more other sensors selected from the group consisting of: accelerometer, inclinometer, gyroscope, strain gauge, or other motion or position sensor; microphone or other sound sensor; thermometer or other temperature sensor; camera or other imaging sensor; optical sensor or optoelectronic sensor; blood pressure sensor; ECG/EKG sensor, heart rate monitor, and/or heart rate sensor; EMG sensor or other muscle activity sensor; GPS sensor, other location sensor, magnetometer, or compass; spectroscopy sensor or other spectral analysis sensor; electrochemical sensor; blood oximetry sensor; piezoelectric sensor; chewing sensor or swallowing sensor; respiration sensor; pressure sensor; galvanic skin response sensor; and taste or odor sensor.

In an example, a monitor can further comprise one or more interface components selected from the group consisting of: a computer-to-human interface such as a display screen, one or more lights, one or more speakers, and/or one or more tactile actuators; a human-to-computer interface such as a touch screen, one or more touch-activated buttons, microphone and speech-recognition capability, and/or gesture recognition capability; one or more accelerometers; one or more cameras; and a GPS component.

I claim:

1. A wearable brain activity monitor comprising a head-worn sensor-positioning member which is configured to position a plurality of electrodes or other brain activity sensors at selected locations on a person's head, wherein the sensor-positioning member snuggly loops around a portion of a lateral perimeter of the person's ear.

2. The monitor in claim 1 wherein the sensor-positioning member loops around a percentage of the lateral perimeter of the person's ear in the range of 50% to 80%.

3. The monitor in claim 1 wherein the sensor-positioning member loops around the person's ear from the 10 o'clock position to the 6 o'clock position.

4. The monitor in claim 1 wherein the sensor-positioning member loops around the person's ear within the range of the 9 o'clock position to the 6 o'clock position.

5. The monitor in claim 1 wherein the monitor comprises two electrodes or other brain activity sensors at locations selected from the following set of placement sites: T7, T8, TP7 and TP8.

6. A wearable brain activity monitor comprising a head-worn sensor-positioning member which is configured to position a plurality of electrodes or other brain activity sensors at selected locations on a person's head, wherein the selected locations are on the person's ear and/or within the person's ear canal.

7. The monitor in claim 6 wherein the sensor-positioning member snuggly loops around a portion of the lateral perimeter of the person's ear and also fits into the person's ear canal.

8. The monitor in claim 6 wherein the sensor-positioning member loops around a percentage of the lateral perimeter of the person's ear in the range of 25% to 70%.

9. The monitor in claim 6 wherein the sensor-positioning member loops around the person's ear from the 9 o'clock position to the 3 o'clock position.

10. The monitor in claim 6 wherein the sensor-positioning member loops around the person's ear within the range of the 9 o'clock position to the 5 o'clock position.

11. The monitor in claim 6 wherein the monitor comprises two electrodes or other brain activity sensors at locations selected from the following set of placement sites: TP7 and TP8.

12. The monitor in claim 6 wherein at least one electrode or other brain activity sensor is configured to be within the person's ear canal.

13. The monitor in claim 6 wherein the monitor is embodied in an ear bud.

14. The monitor in claim 6 wherein the monitor is embodied in a hearing aid.
